SEQUENCE LISTING (21 sequences)

SEQ ID NO: 1 - DNA, 16 bp, synthetic construct
  misc_feature 1..16: note = DBCO-modified AS
  source 1..16: mol_type = other DNA
gctattaggagtcttt

SEQ ID NO: 2 - DNA, 16 bp, synthetic construct
  misc_feature 1..16: note = Fluorescein-labeled DBCO-modified AS
  source 1..16: mol_type = other DNA
gctattaggagtcttt

SEQ ID NO: 3 - DNA, 16 bp, synthetic construct
  misc_feature 1..16: note = Cy3-labeled and DBCO-modified AS
  source 1..16: mol_type = other DNA
gctattaggagtcttt

SEQ ID NO: 4 - DNA, 16 bp, synthetic construct
  misc_feature 1..16: note = Dabcyl-labeled sense
  source 1..16: mol_type = other DNA
aaagactcctaatagc

SEQ ID NO: 5 - DNA, 16 bp, synthetic construct
  misc_feature 1..16: note = Dabcyl-labeled sense
  source 1..16: mol_type = other DNA
aaagactcctaatagc

SEQ ID NO: 6 - DNA, 16 bp, synthetic construct
  misc_feature 1..16: note = Dabcyl-labeled scrambled sense
  source 1..16: mol_type = other DNA
acgactagtatcacaa

SEQ ID NO: 7 - DNA, 16 bp, synthetic construct
  misc_feature 1..16: note = Amine-modified PS AS
  source 1..16: mol_type = other DNA
gctattaggagtcttt

SEQ ID NO: 8 - DNA, 16 bp, synthetic construct
  misc_feature 1..16: note = DBCO-modified PS AS
  source 1..16: mol_type = other DNA
gctattaggagtcttt

SEQ ID NO: 9 - DNA, 16 bp, synthetic construct
  misc_feature 1..16: note = Fluorescein-labeled DBCO-modified PS AS
  source 1..16: mol_type = other DNA
gctattaggagtcttt

SEQ ID NO: 10 - DNA, 16 bp, synthetic construct
  misc_feature 1..16: note = Cy3-labeled and DBCO-modified PS AS
  source 1..16: mol_type = other DNA
gctattaggagtcttt

SEQ ID NO: 11 - DNA, 16 bp, synthetic construct
  misc_feature 1..16: note = Cy5-labeled and DBCO-modified PS AS
  source 1..16: mol_type = other DNA
gctattaggagtcttt

SEQ ID NO: 12 - DNA, 16 bp, synthetic construct
  misc_feature 1..16: note = Fluorescein-labeled amine-modified PS AS
  source 1..16: mol_type = other DNA
gctattaggagtcttt

SEQ ID NO: 13 - DNA, 16 bp, synthetic construct
  misc_feature 1..16: note = Cy3-labeled and amine-modified PS AS
  source 1..16: mol_type = other DNA
gctattaggagtcttt

SEQ ID NO: 14 - DNA, 16 bp, synthetic construct
  misc_feature 1..16: note = Cy5-labeled and amine-modified PS AS
  source 1..16: mol_type = other DNA
gctattaggagtcttt

SEQ ID NO: 15 - DNA, 16 bp, synthetic construct
  misc_feature 1..16: note = PS AS containing amino-modifier (N2-amine modification-C6-dG)
  source 1..16: mol_type = other DNA
gctattaggagtcttt

SEQ ID NO: 16 - DNA, 16 bp, synthetic construct
  misc_feature 1..16: note = Fluorescein-labeled PS AS containing amino-modifier (N2-aminemodification-C6-dG)
  source 1..16: mol_type = other DNA
gctattaggagtcttt

SEQ ID NO: 17 - DNA, 16 bp, synthetic construct
  misc_feature 1..16: note = Cy3-labeled PS AS containing amino-modifier (N2-aminemodification-C6-dG)
  source 1..16: mol_type = other DNA
gctattaggagtcttt

SEQ ID NO: 18 - DNA, 16 bp, synthetic construct
  misc_feature 1..16: note = Cy5-labeled PS AS containing amino-modifier (N2-aminemodification-C6-dG)
  source 1..16: mol_type = other DNA
gctattaggagtcttt

SEQ ID NO: 19 - DNA, 16 bp, synthetic construct
  misc_feature 1..16: note = Scrambled PS
  source 1..16: mol_type = other DNA
atgtccgttgtgtata

SEQ ID NO: 20 - DNA, 16 bp, synthetic construct
  misc_feature 1..16: note = Dabcyl-labeled sense
  source 1..16: mol_type = other DNA
aaagactcctaatagc

SEQ ID NO: 21 - DNA, 16 bp, synthetic construct
  misc_feature 1..16: note = DBCO-modified scrambled PO LNA
  source 1..16: mol_type = other DNA
ggctactacgccgtca

(19) United States
(12) Patent Application Publication
(10) Pub. No.: US 2025/0135013 A1
(43) Pub. Date: May 1, 2025

(54) Targeting Oncogenic KRAS with Molecular Brush-Conjugated Antisense Oligonucleotide

(71) Applicant: Northeastern University, Boston, MA (US)

(72) Inventors: Ke Zhang, Natick, MA (US); Dali Wang, Boston, MA (US)

(21) Appl. No.: 18/683,235

(22) PCT Filed: Aug. 19, 2022
(86) PCT No.: PCT/US2022/075240

§ 371 Date: Feb. 12, 2024

Related U.S. Application Data
(60) Provisional application No. 63/234,847, filed on Aug. 19, 2021.

(51) Int. Cl. A61K 47/60; A61P 35/00; C12N 15/113

(52) U.S. Cl. CPC A61K 47/60; A61P 35/00; C12N 15/1135; C12N 2310/11; C12N 2310/351

(57) ABSTRACT

Provided herein are, in various embodiments, methods and compositions comprising polyethylene glycol (PEG)-conjugated oligonucleotides (e.g., anti-sense oligonucleotides) for treatment of cancer. In certain embodiments, the disclosure provides for methods and compositions for enhancing efficacy of anti-sense oligonucleotides. In still further embodiments, the disclosure provides methods and compositions for treatment of non-small cell lung cancer.

### RELATED APPLICATION

This application claims the benefit of U.S. Provisional Application No. 63/234,847, filed on Aug. 19, 2021. The entire teachings of the above application are incorporated herein by reference.

### GOVERNMENT SUPPORT

This invention was made with government support under 1R01CA251730 and R01GM121612 from the National Institutes of Health and under 2004947 from the National Science Foundation. The government has certain rights in the invention.

### INCORPORATION BY REFERENCE OF MATERIAL IN XML

This application incorporates by reference the Sequence Listing contained in the following extensible Markup Language (XML) file being submitted concurrently herewith:
a File name: 52002342001.xml; created Aug. 19, 2022, 27,664 Bytes in size.

### BACKGROUND

Mutationally activated RAS genes (HRAS, KRAS, and NRAS) are the most frequently mutated proto-oncogenes in human cancer (27%), with KRAS being the most mutated oncogene (85% of all RAS missense mutations). KRAS functions as a molecular switch, cycling between guanosine triphosphate (GTP)-bound (on) and guanosine diphosphate (GDP)-bound (off) states to affect intracellular signaling through cell surface receptors. The missense mutation of KRAS aberrantly activates the protein into a hyperexcitable state by attenuating its guanosine triphosphatase (GTPase) activity, which results in an accretion of GTP-bound, activated KRAS and persistent activation of downstream signaling pathways.

Mutations of KRAS are associated with poor prognosis in several cancers, and a substantial body of evidence has confirmed the role of KRAS in the initiation and maintenance of cancer, thus making KRAS an important therapeutic target.

Thus, RAS inhibition and the development of novel therapies are important clinical needs.

### SUMMARY

In one aspect of the disclosure, there is provided a method of inhibiting cancer in a subject in need thereof, said method comprising administering to the subject a composition comprising: a polyethylene glycol (PEG)-conjugated antisense oligonucleotide (ASO). In one aspect of the disclosure, there is provided a method of inhibiting or reducing tumor growth in a subject, said method comprising administering to the subject an effective amount of a pacDNA comprising a plurality of anti-sense oligonucleotides (ASOs) that specifically binds an oncogene. In some embodiments, the oncogene is the KRAS gene, which encodes the K-Ras protein. In some embodiments, the subject has non-small cell lung cancer (NSCLC). In some embodiments, the ASOs are identical in nucleotide sequence, and in other embodiments the plurality of ASOs comprises anti-KRAS oligonucleotides of different nucleotide sequences (i.e., the oligonucleotides share less than 100% sequence identity).

In one aspect of the disclosure, there is provided a method of inhibiting a KRAS-mediated disease or disorder in a subject in need thereof, said method comprising administering to the subject a composition comprising: a polyethylene glycol (PEG)-conjugated antisense oligonucleotide (ASO).

In one aspect of the disclosure, there is provided a method of downregulating KRAS in a subject in need thereof, said method comprising administering to the subject a composition comprising: a polyethylene glycol (PEG)-conjugated antisense oligonucleotide (ASO).

In one aspect of the disclosure, there is provided a method the enhancing the delivery of conjugated ASOs, e.g., for suppressing oncogenic KRAS in vivo, comprising administering to the subject a composition comprising: a polyethylene glycol (PEG)-conjugated antisense oligonucleotide (ASO).

In one aspect of the disclosure, the methods herein reduce the dosage level required for a phenotypic response to administered conjugated ASPs compared with administration of naked ASOs to a subject.

In one aspect of the disclosure, there is provided a composition (e.g., a pharmaceutical composition) comprising a polyethylene glycol (PEG)-conjugated antisense oligonucleotide (ASO).

In one aspect of the disclosure, there is provided a pacDNA comprising a plurality of antisense oligonucleotides (ASOs) that specifically bind an oncogene. In some embodiments, the oncogene is the KRAS gene. In some embodiments, the ASOs are identical in nucleotide sequence, and in other embodiments the plurality of ASOs comprises anti-KRAS oligonucleotides of different nucleotide sequences.

In one aspect of the disclosure, some or all of ASOs in the composition can be natural, chemically modified, have a conjugation site at the sequence terminus, have a conjugation site at internal position, and be stable or be bioreductively cleavable (see, e.g., FIGS. 1B, 1C, and 1D; Table 1).

In one aspect of the disclosure, there is provided a conjugate, e.g., a conjugate comprising a polyethylene glycol (PEG) conjugated to an antisense oligonucleotide (ASO). In another aspect, a pacDNA structure is provided, wherein the structure comprises one or more (e.g., two, three, four, or more) ASO strands. In another aspect, the conjugate is a bottlebrush polymer-locked nucleic acid (pacLNA) conjugate.

In one aspect of the disclosure, there is provided a delivery system, e.g., a nucleic acid delivery system, comprising one or more conjugates or compositions described herein.

In one aspect of the disclosure, the systems, conjugates and/or compositions are administered for disease management, e.g., chronic disease management.

In one aspect of the disclosure, methods of making the systems, structures and compositions descried herein are provided.

In one aspect of the disclosure, there is provided a kit, comprising one or more ASO or composition described herein and, optionally, a container and/or instructions.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A shows the antisense-targeted region of the KRAS mRNA. Highlighted deoxyguanosine (underlined) in the KRAS ASO sequence is used for mid-sequence conjugation to the bottlebrush polymer. FIG. 1B shows sample ID and chemical structure. Clv: cleavable; m: mid-sequence conjugation; PO: phosphodiester; PS: phosphorothioate; _{Y}PEG: Polymer 2. FIG. 1C shows a structural model from a coarse-grained molecular dynamics simulation of the pacDNA. A crystal structure of recombinant human DNase I is shown to the left of the pacDNA for size comparison. FIG. 1D shows polymer, ASO, and linker chemistry. FIG. 1E shows synthesis of pacDNA chemistry. FIG. 1F shows a ¹H nuclear magnetic resonance (NMR) spectrum of bottlebrush polymer in CDCl₃. FIG. 1G shows a N,N-dimethylformamide (DMF) GPC chromatogram of the bottlebrush polymer.

FIG. 2A shows aqueous GPC chromatograms of free ASO, 40 kDa Y-shaped PEG-ASO conjugate, and PO pacDNA. FIG. 2B shows agarose gel electrophoresis (1%) of pacDNAs, 40 kDa Y-shaped PEG-ASO conjugate, and free ASOs. FIG. 2C shows Zeta (2) potential measurements of pacDNAs and controls in Nanopure™ water. FIG. 2D shows TEM images of pacDNA (negatively stained with 2% uranyl acetate). FIG. 2E shows a particle size histogram of pacDNA determined by analyzing 400+ individual particles from TEM images. FIG. 2F shows DLS number-average size distribution of PO pacDNA. FIG. 2G shows reductive release of free ASO from PS pacDNA_{Clv} and PS pacDNA_{m,Clv} after treatment with 10 mM DTT for 1 h. The PS pacDNA and PS pacDNAₘ were non-cleavable and thus showed no release of free ASO. FIG. 2H shows schematics of enzymatic digestion kinetics assay based upon fluorophore- and quencher-tagged DNA duplex. FIG. 2I shows DNA hybridization kinetics for pacDNAs and controls. FIG. 2J shows DNase I degradation kinetics for pacDNAs and controls.

FIG. 3A-1 shows flow cytometry measurement of NCI-H358 cells treated with Cy3-labeled free PO ASO for 4 h. FIG. 3A-2 shows flow cytometry measurement of NCI-H358 cells treated with PO pacDNA (250-5000 nM; ASO-basis) for 4 h. FIG. 3A-3 shows flow cytometry measurement of NCI-H358 cells treated with Cy3-labeled free PS ASO for 4 h. FIG. 3A-4 shows flow cytometry measurement of NCI-H358 cells treated with PS pacDNA (250-5000 nM; ASO-basis) for 4 h. FIG. 3B shows cellular uptake in NCI-H358 cells as indicated by mean cellular fluorescence, showing a “leveling” effect where the bottlebrush polymer reduces the uptake of normally high-uptake ASO but boosts that of low-uptake ASO. FIG. 3C shows confocal microscopy of NCI-H358 cells treated with fluorescently labeled PO or PS forms of molecular ASO and pacDNA. FIG. 3D shows dose-dependent depletion of KRAS in NCI-H358 cells by pacDNAs. FIG. 3E shows a Western blot analysis of MAPK signaling in NCI-H358 cells after treatment with pacDNAs for 72 h. FIG. 3F-1 shows an inhibitive effect in the proliferation of KRAS mutant cells (NCI-H358) by KRAS depletion using free ASOs and pacDNAs. FIG. 3F-2 shows an inhibitive effect in the proliferation of wild-type cells (PC9) by KRAS depletion using free ASOs and pacDNAs. For FIGS. 3F-1 and 3F-2, statistical analysis was performed using two-way ANOVA with Sidak's multiple comparison testing (****P<0.0001).

FIG. 4A-1 shows flow cytometry measurement of NCI-H358 cells treated with Cy3-labeled _{Y}PEG-PS ASO for 4 h (total cell count: 10,000). FIG. 4A-2 shows flow cytometry measurement of NCI-H358 cells treated with Cy3-labeled PS pacDNAₘ (250-5000 nM; ASO-basis) for 4 h (total cell count: 10,000). FIG. 4A-3 shows flow cytometry measurement of NCI-H358 cells treated with Cy3-labeled PS pacDNA_{m,Clv} (250-5000 nM; ASO-basis) for 4 h (total cell count: 10,000). FIG. 4A-4 shows flow cytometry measurement of NCI-H358 cells treated with Cy3-labeled PS pacDNA_{Clv} (250-5000 nM; ASO-basis) for 4 h (total cell count: 10,000). FIG. 4B shows flow cytometry mean fluorescence as a function of incubation concentration. Cells were treated in serum-free media. The data for free ASOs in FIG. 3B are reproduced here for comparison. FIG. 4C shows flow cytometry mean fluorescence as a function of incubation concentration. Cells were treated in media containing 10% FBS. The data for free ASOs in FIG. 3B are reproduced here for comparison.

FIG. 5A-1 shows flow cytometric analysis of NCI-H358 cells fed with PO pacDNA in the presence of various pharmacological endocytosis inhibitors (MβCD: ethyl-β-cyclodextrin; CPM: chlorpromazine). FIG. 5A-2 shows flow cytometric analysis of NCI-H358 cells fed with PS pacDNA in the presence of various pharmacological endocytosis inhibitors (MβCD: ethyl-β-cyclodextrin; CPM: chlorpromazine). FIG. 5A-2 shows flow cytometric analysis of NCI-H358 cells fed with PS ASO in the presence of various pharmacological endocytosis inhibitors (MβCD: ethyl-β-cyclodextrin; CPM: chlorpromazine). For FIGS. 5A-1, 5A-2, and 5A-3 statistical significance was calculated using one-way ANOVA with Dunnett's multiple comparison testing (****P<0.0001, ***P<0.001, **P<0.01, *P<0.05). FIG. 5B shows Dose-dependent response of KRAS protein levels in NCI-H358 cells after treatment with PS pacDNAₘ or PS pacDNA_{m,Clv}, as determined by Western blot analysis. Gene knockdown levels (determined by gel densitometry analysis) are shown as fractions below the gel image. FIG. 5C shows a Western blot analysis of KRAS protein levels in the lysates of PC9 cells (wild-type KRAS) after treatment with free ASOs, pacDNAs, and controls. Gene knockdown levels (determined by gel densitometry analysis) are shown as fractions below the gel image.

FIG. 6A shows cell apoptosis following sample treatment determined by annexin V/propidium iodide staining. Living, early apoptotic, and late apoptotic cell populations (%) are shown in the lower left, lower right, and upper right quadrants, respectively. Results are representatives of three independent experiments. FIG. 6B shows a Western blot analysis of pro-caspase 3 protein after treatment with free ASOs or pacDNAs. FIG. 6C shows viability of NCI-H358 cells in the presence of PS pacDNA_{Clv}, PS pacDNAₘ, PS pacDNA_{m,Clv}, or the bottlebrush polymer.

FIG. 7A shows plasma pharmacokinetics of pacDNAs, free ASO (both in PO and PS forms), and the bottlebrush polymer in C57BL/6 mice. FIG. 7B shows fluorescence monitoring of i.v. injected Cy5-labeled pacDNAs and controls in BALB/c-nu mice bearing NCI-H358 xenograft. FIG. 7C shows ex vivo imaging of tumors and other major organs 14- or 91-days post injection. Imaging settings were kept identical. FIG. 7D shows confocal microscopy of cryosectioned tumor tissue 24 h post-injection, showing tumor penetration (PS pacDNA). Statistical analysis was performed using two-way ANOVA with Tukey's multiple comparison testing. ****P<0.0001.

FIG. 8A shows fluorescence imaging of BALB/c nude mice bearing a human lung NCI-H358 xenograft following intravenous injection of Cy5-labeled samples and controls. Panel to the bottom: ex vivo imaging of tumors and other major organs 24 h post injection. FIG. 8B shows confocal images of NCI-H358 tumor cryosections 24 h after intravenous injections of PO pacDNA or brush polymers. Cy5-labeled ASO (in PO pacDNA) or bottlebrush polymer; nucleus staining with Hoechst 33342. FIG. 8C shows daily fluorescence monitoring of BALB/c nude mice bearing a human lung NCI-H358 xenograft following a single intravenous injection of Cy5-labeled pacDNAs or bottlebrush polymer for 2 weeks. FIG. 8D shows continued weekly fluorescence monitoring of BALB/c nude mice bearing a human lung NCI-H358 xenograft following a single intravenous injection of Cy5-labeled bottlebrush polymer for 13 weeks.

FIG. 9A shows NCI-H358 tumor volume changes in 36 days with i.v. administration of PBS, ASOs, and pacDNAs at equivalent ASO doses (0.5 μmol/kg) every third day (treatment started on day 0). FIG. 9B shows Kaplan-Meier endpoint animal survival analysis for the NCI-H358 xenograft study. Data are shown as the percentage of remaining animals with tumors <4× the initial starting volume in each treatment group. FIG. 9C shows tumor growth inhibition of NCI-H358 xenografts at a reduced ASO dosage (0.1 μmol/kg). FIG. 9D shows immunohistostaining of tumor cryosections, showing reduced KRAS expression in pacDNA-treated groups (top row) and shows hematoxylin and eosin staining of tumor tissues after the treatment period (bottom row). FIG. 9E shows NCI-H1944 tumor volume changes with i.v. administration of PBS, PO pacDNA, and PS pacDNA at equivalent ASO doses (2.0 μmol/kg) every third day (treatment started on day 0). FIG. 9F shows Kaplan-Meier survival curves for NCI-H1944 tumor-bearing mice. Data are shown as the percentage of remaining animals with tumors <4× the initial starting volume in each treatment group. FIG. 9G shows body weight changes of NCI-H1944 tumor-bearing mice during the treatment period. For tumor inhibition, statistical analysis was performed using two-way ANOVA with Tukey's multiple comparison testing. For animal survival analysis, statistical significance was calculated by the log-rank test. ****P<0.0001, ***P<0.001, **P<0.01, *P<0.05.

FIG. 10A shows tumor volume changes in 36 days with intravenous administration of PS pacDNA_{Clv} or PS pacDNA_{m,Clv} at an ASO dosage of 0.5 μmol/kg every third day (treatment started on day 0). Statistical significance was calculated by two-way ANOVA with Tukey's multiple comparison testing (****P<0.0001). FIG. 10B shows endpoint animal survival analysis. Data are shown as the percentage of remaining animals with tumors <4× the initial starting volume in each treatment group. Statistical analysis was performed using the log-rank test (**P<0.01, *P<0.05). FIG. 10C shows histological analysis of tumor slices 36 days after treatment, showing reduced KRAS expression by immunohistochemistry staining (top row) and haematoxylin and eosin (H&E) staining (bottom row). The data for the vehicle (PBS) treatment group are reproduced from FIG. 9 for comparison. FIG. 10D shows additional immunohistostaining images of tumor cryosections, showing reduced KRAS expression in pacDNA-treated groups vs control across the entire tumor section. Scale bar, 2 mm. FIG. 10E shows a Western blot analysis of KRAS protein level after treatment with pacDNAs. FIG. 10F shows cell viability after treatment of PS ASO and pacDNAs measured by MTT cytotoxicity assay. A more pronounced response is observed for the pacDNAs compared to the PS ASO. Statistical analysis was performed using two-way ANOVA with Sidak's multiple comparison testing (****P<0.0001). FIG. 10G shows representative histological staining of NCI-H1944 tumors after 27-day treatment with pacDNAs or vehicle immunohistostaining of KRAS in NCI-H1944 tumor slices (top row) and hematoxylin and eosin staining of the same tumor (bottom row).

FIG. 11A shows body weight changes for NCI-H358 xenograft-bearing mice (dosage: 0.5 μmol/kg; ASO basis). Data are given as mean±s.d; Statistical significance was calculated using two-way ANOVA with Tukey's multiple comparison test. No statistical difference was detected between groups. FIG. 11B shows body weight changes for NCI-H358 xenograft-bearing mice (dosage: 0.1 μmol/kg; ASO basis). Data are given as mean±s.d; Statistical significance was calculated using two-way ANOVA with Tukey's multiple comparison test. No statistical difference was detected between groups. FIG. 11C shows microscopic images of haematoxylin and eosin (H&E)-stained sections of various organs after pacDNA treatment. NCI-H358 xenograft-bearing mice were treated for a 36-day period with pacDNAs and controls at an ASO dosage of 0.5 μmol/kg. No apparent histological anomalies were detected. FIG. 11D shows microscopic images of haematoxylin and eosin (H&E)-stained sections of various organs after pacDNA treatment. NCH-H358 xenograft-bearing mice were treated for a 36-day period with pacDNAs and controls at an ASO dosage of 0.1 μmol/kg. No apparent histological anomalies were detected. FIG. 11E shows microscopic images of haematoxylin and eosin (H&E)-stained sections of various organs after pacDNA treatment. NCI-H1944 xenograft-bearing mice were treated for a 27-day period with pacDNAs and controls at an ASO dosage of 2.0 μmol/kg. No apparent histological anomalies were detected.

FIG. 12A shows hemolysis of human blood (type O+) treated with pacDNA and controls, as determined by spectrophotometric measurement of hemoglobin present in the supernatant of centrifuged RBC suspensions. The % RBC hemolysis is defined as the percentage of hemoglobin present in the supernatant compared with the total hemoglobin released by Triton X-100 treatment. Inset: photograph of centrifuged RBC suspensions. Sample identity for pacDNAs: 4. PO pacDNA, 5. PS pacDNA, 6. PS pacDNA_{Clv}, 7. PS pacDNAₘ, 8. PS pacDNA_{m, Clv}. FIG. 12B shows anti-PEG IgM levels in the serum of C57BL/6 mice after repeated injections of pacDNAs and controls at timed intervals. FIG. 12C shows anti-PEG IgG levels in the serum of C57BL/6 mice after repeated injections of pacDNAs and controls at timed intervals. FIG. 12D shows selected cytokine levels in the serum in C57BL/6 mice following injection of pacDNAs or controls. FIG. 12E shows repeated plasma pharmacokinetics measurements after four sequential i.v. administration of pacDNAs or free bottlebrush polymer in C57BL/6 mice. Plasma ASO or polymer levels were monitored after each injection. Statistical analysis was performed using one-way ANOVA with Tukey's multiple comparison testing. ****P<0.0001, ***P<0.001, **P<0.01, *P<0.05.

FIG. 13A shows blood biochemistry analysis. Healthy C57BL/6 mice (6-8 weeks, n=4) were injected i.v. with PO pacDNA, PS pacDNA, PS ASO, and free bottlebrush polymer three times a week for two weeks with the equal DNA or brush polymer dose of 0.5 μmol/kg animal weight. Blood samples were collected from the submandibular vein 24 h after last injection, allowed to clot by being left undisturbed for 30 min, and centrifuged at 3000 rpm for 5 min, and the serum was collected. The measurements were performed by the Comparative Pathology Laboratory of the MIT Division of Comparative Medicine. 1. Control, 2. PS ASO, 3. PO pacDNA, 4. PS pacDNA, 5. Brush polymer. FIG. 13B shows IFN-γ and IL-4 levels in the serum in C57BL/6 mice 2 h after the treatment with pacDNAs and controls. Statistical significance was calculated using one-way ANOVA with Tukey's multiple comparison testing. No statistical difference was detected between groups. FIG. 13C shows anti-PEG IgM levels in the serum of C57BL/6 mice after the animals were given 12 i.v. doses of pacDNAs and controls over 36 days. Statistical significance was calculated using one-way ANOVA with Tukey's multiple comparison testing. ****P<0.0001, ***P<0.001, **P<0.01, *P<0.05. FIG. 13D shows anti-PEG IgG levels in the serum of C57BL/6 mice after the animals were given 12 i.v. doses of pacDNAs and controls over 36 days. Statistical significance was calculated using one-way ANOVA with Tukey's multiple comparison testing. ****P<0.0001, ***P<0.001, **P<0.01, *P<0.05.

FIG. 14A shows chemical structures of pacLNAs. FIG. 14B shows aqueous GPC chromatograms of PO LNA and PO pacLNA. FIG. 14C shows TEM image of pacLNA (negatively stained with 2% uranyl acetate). FIG. 14D show DLS number-average size distribution of PO pacLNA. FIG. 14E shows Zeta (ζ) potential measurements of free LNAs and pacLNAs in Nanopure™ water. FIG. 14F shows the synthesis of pacLNA. FIG. 14G shows N,N-dimethylformamide (DMF) GPC chromatogram of the bottlebrush polymer. FIG. 14H shows aqueous GPC chromatogram of PS LNA and PS pacLNA. FIG. 14I shows an additional TEM image of pacLNA. FIG. 14J shows size distribution of pacLNA measured from TEM images. A minimum of 300 particles were measured.

FIG. 15A shows hybridization kinetics for pacLNAs and controls. FIG. 15B shows DNase I degradation kinetics for pacLNAs and controls.

FIG. 16A-1 shows flow cytometry measurements of NCI-H358 cells treated with Cy3-labeled PO LNA (0.25-5 μM; ASO basis). FIG. 16A-2 shows flow cytometry measurements of NCI-H358 cells treated with Cy3-labeled PS LNA (0.25-5 μM; ASO basis). FIG. 16A-3 shows flow cytometry measurements of NCI-H358 cells treated with Cy3-labeled PO pacLNA (0.25-5 μM; ASO basis). FIG. 16A-4 shows flow cytometry measurements of NCI-H358 cells treated with Cy3-labeled PS pacLNA (0.25-5 μM; ASO basis). FIG. 16B shows confocal microscopy of NCI-H358 cells treated with Cy3-labeled LNAs and pacLNAs. Scale bar, 20 μm. FIG. 16C shows cellular uptake level in NCI-H358 cells as indicated by mean fluorescence intensity. FIG. 16D shows cell viability test of NCI-H358 cells after treatment with LNAs, pacLNAs and brush polymer. Statistical significance was calculated using Student's two-tailed t test. **P<0.01, ***P<0.001. FIG. 16E shows a Western blot analysis of KRAS depletion in NCI-H358 cells after treatment with LNA and pacLNAs for 72 h. FIG. 16F shows confocal images of NCI-H358 cells after treated with LNAs and pacLNAs (5 μM) for 4 h under different laser power. Scale bar, 20 μm.

FIG. 17A shows plasma pharmacokinetics of LNAs, pacLNAs and bottlebrush polymer in C57BL/6 mice. Statistical significance was calculated using two-way ANOVA. ****P<0.0001. FIG. 17B shows fluorescence images of dissected tumor and major organs 56 d or 91 d post intravenous injection. FIG. 17C shows long-term live mice imaging of NCI-H358 tumor-bearing mice after one single intravenous injection of Cy5-labeled LNAs, pacLNAs and bottlebrush polymer. Image setting were kept identical. FIG. 17D shows fluorescence imaging of athymic mice bearing human lung NCI-H358 xenograft following intravenous injection of Cy5-labeled LNAs, pacLNAs and brush polymer. FIG. 17E shows daily fluorescence monitoring of athymic mice bearing human lung NCI-H358 xenograft following a single intravenous injection of Cy5-labeled LNAs, pacLNAs or brush polymer for 2 weeks. FIG. 17F shows continued weekly fluorescence monitoring of athymic mice bearing human lung NCI-H358 xenograft following a single intravenous injection of Cy5-labeled LNAs, pacLNAs or brush polymer for up to 13 weeks. FIG. 17G shows confocal microscopy of cryosectioned tumor tissue 24 h post-injection.

FIG. 18A shows NCI-H358 tumor volume changes in 36 days with weekly intravenous administration of vehicle and pacLNAs. *P<0.1, **P<0.01, ***P<0.001, ****P<0.0001. Statistical analysis was performed using Student's two-tailed t test. FIG. 18B shows Kaplan-Meier endpoint animal survival analysis for the NCI-H358 xenograft study. Data are shown as the percentage of remaining animals with tumors <4× the initial starting volume in each treatment group. *P<0.1, **P<0.01. Statistical analysis was performed using Mantel-Cox tests. FIG. 18C shows body weight changes for NCI-H358 xenograft-bearing mice. FIG. 18D shows immunohistostaining of tumor cryosections, showing reduced KRAS expression in pacLNA-treated groups. FIG. 18E shows a Western blotting analysis of tumor tissues. FIG. 18F shows additional immunohistostaining images of tumor cryosections. FIG. 18G shows microscopic images of hematoxylin and eosin (H&E)-stained sections of various organs after pacLNA treatment. No apparent histological anomalies were detected.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines, and animals. The present disclosure is not limited by the illustrated ordering of acts or events, as some acts may occur in different orders and/or concurrently with other acts or events. Furthermore, not all illustrated acts, steps, or events are required to implement a methodology in accordance with the present disclosure. Many of the techniques and procedures described, or referenced herein, are well understood and commonly employed using conventional methodology by those skilled in the art.

Unless otherwise defined, all terms of art, notations, and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an,” and “the” should be understood to include plural reference unless the context clearly indicates otherwise.

Throughout this specification and the claims which follow, unless the context requires otherwise, the word “comprise,” and variations such as “comprises” and “comprising,” will be understood to imply the inclusion of, e.g., a stated integer or step or group of integers or steps, but not the exclusion of any other integer or step or group of integers or steps. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary the scope of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

KRAS has long been considered undruggable due to the lack of deep binding pockets. However, Moore et al. (Nat. Rev. Drug Discov. 19, 533-552 (2020)) and Ostrem et al. (Nature 503, 548-551 (2013)) demonstrated that the cysteine residue of the G12C mutant gives rise to a new pocket that can be selectively targeted by small-molecule binders. This development led to the accelerated approval of sotorasib and shortly thereafter adagrasib, the first-in-class drug KRAS inhibitors for advanced non-small cell lung carcinoma (NSCLC).

The breakthrough therapy designation of both compounds speaks to the significance of the target. Nonetheless, the G12C mutation only occurs in a small percentage of KRAS^{MUT} cancers—predominantly in lung adenocarcinomas and, at a lower frequency, in colorectal cancer and pancreatic ductal adenocarcinomas (44%, 11%, and 3%, respectively). Thus, RAS inhibition and the development of novel therapies remain unmet clinical needs.

The difficulty in developing small molecule inhibitors for KRAS has heightened the importance of alternative methods targeting the oncogene, for example using antisense oligonucleotides (ASOs), which offer a possibility to yield drugs for targets that have proven to be intractable to traditional drug modalities. As used herein in reference to ASOs, the term “specifically binds” means an ASO reacts or associates or binds to a target nucleic acid sequence more frequently, more rapidly, with greater duration, with greater affinity, or combinations of the above, than with alternative sequences, including unrelated nucleic acid sequences.

Nucleic acid drugs are attractive for traditionally undruggable targets due to their ability to selectively bind with human or pathogen transcriptome to knock down gene expression, to alter mRNA splicing, to target trinucleotide repeat disorders, to affect non-coding RNAs (ncRNAs) involved in transcriptional and epigenetic regulation, to upregulate target genes, and to edit the genome. To date, fifteen oligonucleotide drugs have been approved by the U.S. Food and Drug Administration (FDA), six of which were approved since 2019.

Chemical modification represents the most effective strategy to address the limitations associated with ASO therapeutics among the current ASO drug delivery strategies. Phosphorothioate (PS) backbone modification, the first generation of chemically modified ASOs enhances the nuclease stability and facilitates the cellular uptake by providing a strong binding of ASO with plasma protein. Later, 2′ position modifications of the ribose sugar, including 2′-O-methoxyethyl (2′-MOE), 2′-O-methyl (2′-OMe) and 2′-Fluoro (2′-F) were developed to enhance the binding affinity of ASOs and improve their stability in plasma. Bridged nucleic acids, such as locked nucleic acid (LNA), constrain the ribose sugar in the 3′-endo conformation, thus largely enhancing the binding affinity of ASO towards its target and also improving its nuclease stability. Most U.S. Food and Drug Administration (FDA)-approved ASO drugs incorporate several chemical modifications, e.g., Nusinersen, which is a 18mer PS 2′-MOE modified ASO approved in 2016 for treating spinal muscular atrophy.

Despite these clinical advances, nucleic acid drugs are being mainly developed for rare diseases originating from the liver, or in tissues that can be treated by local injection, such as the spinal cord or the eye. The limited use cases and overall slow bench-to-bedside translation reflect the intrinsic difficulties associated with oligonucleotide drugs. Unmodified, naked oligonucleotides are easily degraded by nucleases, can undergo rapid renal and hepatic clearance, and are incapable of cellular uptake owing to a combination of hydrophilicity and high molecular weight. Advanced delivery systems (e.g., polycationic polymers, nanoparticles, liposomal formulations, etc.) have been developed to overcome these difficulties. However, other than liposomes, most carrier systems still need to be proven relevant in a clinical setting.

Challenges include toxicity, immunogenicity, consistency in formulation, chemical and in vivo stability, release control, and problems associated with large-scale manufacturing. On the other hand, nucleic acid modification chemistries have been far more successful for clinical translation, with all currently approved oligonucleotide drugs adopting one or more forms of modifications. For example, the phosphorothioates (PS) have greatly improved enzymatic stability, potency, and duration of oligonucleotides in vivo, making it possible to bypass the need for complex carriers. However, although nonspecific binding between PS and serum proteins improves tissue uptake and reduces renal clearance, the blood pharmacokinetics of PS drugs remains very poor.

The liver and the kidney are often the organs that receive most of the injected dose, followed by the bone marrow, adipocytes, and lymph nodes. To achieve a therapeutically relevant concentration at tumor tissues, the dosage often exceeds safety tolerances. In fact, PS show increased potential for non-specific adverse effects including induction of stress responses, prolongation of activated partial thromboplastin time (aPTT), thrombocytopenia, and increased serum transaminase activities. Mipomersen, the first systemically administered PS drug that treats homozygous familial hypercholesterolemia, was only approved in the US and not Europe due to concerns of adverse toxic effects. Therefore, a safe, simple, and efficient nucleic acid delivery system that can improve nuclease stability, address non-liver organs, and minimize off-target effects may prove to be the important missing link between oligonucleotides and their adoption for cancer treatment.

Although exhibiting great potential as effective gene therapeutics, the translation of chemically modified ASO therapeutics into the clinic is still largely hindered. Most ASO therapeutics have been developed to target rare diseases through local delivery, such as the eye or spinal cord. Systemic administration usually leads to the accumulation of ASOs in the liver, followed by the kidney and spleen. The delivery challenges hinder the therapeutic potential of ASO to treat common diseases such as cancer.

Furthermore, chemically modified ASOs experience poor pharmacokinetics properties, insufficient tissue delivery and short in vivo half-lives. These shortcomings require frequent and large amounts of chemically modified ASOs. Several studies have revealed that large doses of fully or partially modified ASOs remain as an issue. For example, PS modification increases the non-specific binding between ASO and protein, e.g., the paraspeckle proteins would be delocalized to nucleoli through interaction with PS ASO, leading to the toxicity. Swayze et al. (Nucleic acids research, 35 (2), 687-700) reported that LNA modifications, although showing a stronger potency compared to other modifications, exhibited severe hepatoxicity under a frequent and large amount of dosage. To address these challenges of chemically modified ASOs, a safe and highly efficient delivery system needs to be explored.

Recently, a form of PEGylated oligonucleotides, termed polymer-assisted compaction of DNA (pacDNA), which consists of a small number of ASOs (typically 1-5) tethered to the backbone of a bottlebrush PEG, e.g., via the 3′, 5′, or an internal position of the ASO, has been developed. In some embodiments, the PEGylated oligonucleotides and/or pacDNA are described in U.S. Pat. Nos. 10,590,414; 11,104,901; and US Patent Application No. 2018-0369142 (the contents of each of which is herein incorporated by reference in their entirety). The bottlebrush architecture of the pacDNA conceals the ASO within an intermediate-density PEG environment, which provides the ASO with steric-based selectivity: hybridization with a complementary strand is unaffected, but access by proteins, which are much larger in cross-section diameter, is significantly hindered. Such selectivity reduces enzymatic degradation and most unwanted side effects stemming from specific or non-specific oligonucleotide-protein interactions (e.g., coagulopathy and unwanted immune system activation), while substantially improving the plasma pharmacokinetics (PK) and concentration in non-liver organs. The observed physiochemical and biopharmaceutical enhancements over naked nucleic acids are realized using predominantly PEG, which is generally regarded as safe for therapeutic applications.

Lu et al. (Journal of the American Chemical Society, 138 (29), 9097-9100) reported a bottlebrush polyethylene glycol (PEG) polymer, termed pacDNA (polymer-assisted compaction of DNA) that can serve as a delivering vector for ASOs. In some embodiments, the PEGylated oligonucleotides and/or pacDNA are described in U.S. Pat. Nos. 10,590,414; 11,104,901; and US Patent Application No. 2018-0369142 (the contents of each of which is herein incorporated by reference in their entirety). The densely packed PEG environment hinders the interaction between ASO and protein, while allowing it to hybridize with its target. Such unique architecture and selectivity improve the enzymatic stability of pacDNA and reduce many adverse effects associated with ASO-protein interactions, such as immune system activation. These characteristics lead to enhanced biopharmaceutical properties including improved plasma pharmacokinetics, uptake by non-liver organs and accumulations at tumor.

A clinical validation for targeting KRAS has emerged for the treatment of cancer, but other than the G12C mutant, KRAS has remained undruggable. Methodologies to deplete oncogenic KRAS using nucleic acids and derivatives such as ASO and siRNA molecules have been developed. However, these approaches are limited by inefficient delivery, resulting in increased dosage requirements and side effects associated with off-target binding, unnatural nucleotide analogues, and unwanted immune system activation. Herein, the present disclosure provides compositions and methods comprising pacDNAs demonstrating that the molecular brush-conjugated ASO against KRAS mRNA markedly increases the potency of the ASO in vivo while suppressing nearly all side effects, which critically elevates the translational potential of the antisense approach to the KRAS problem.

Three unique properties of the pacDNA are important for its clinical feasibility. First, the pacDNA is a selective form of oligonucleotide therapeutics. Unlike traditional ASO delivery systems, the pacDNA is a molecular agent that remains hybridizable to target strands without the ASO being separated from the polymer. As detailed herein, the binding kinetics and thermodynamics of pacDNA structures are almost indistinguishable from that of free DNA. Thus, the pacDNA is akin to a selective form of DNA that resists protein binding than a traditional drug delivery vehicle. Because almost all cases of unwanted, non-antisense side effects are preceded by protein recognition of the oligonucleotide, be it degradation, TLR activation, and inhibition of the coagulation cascade, the selectivity of the pacDNA translates into greater in vivo efficiencies with reduced potential for adverse effects. Second, the pacDNA simultaneously enhances transfection efficiency and in vivo properties. Conventional vectors often face an activity-toxicity dilemma: efforts to improve cellular transfection efficiency frequently result in poorer biopharmaceutical properties such as increased uptake by the mononuclear phagocyte system (MPS), clearance, and toxicity. The pacDNA, in contrast, resists opsonization and is not strongly recognized by phagocytic cells, allowing for significantly improved plasma PK and biodistribution parameters, including elimination half-life, blood availability, and passive targeting of non-liver parenchymal organs. In addition, the pacDNA exhibits a moderate level of cellular uptake and reasonable antisense potency. This combination allows the pacDNA to be used at a much lower dosage, which provides flexibility in designing effective therapeutic oligonucleotides by circumventing toxicity constraints. Third, the pacDNA is designed with safety and clinical translatability first and foremost. The core of the pacDNA is a noncationic bottlebrush polymer consisting mainly of the widely used, biocompatible polymer, PEG, which is recognized as generally safe for pharmaceutical use. A novel mechanism of steric compaction (as opposed to complexation, encapsulation, or chemical modification) is used to protect the oligonucleotide and facilitate delivery, which annuls the potential negative effects associated with polycationic, liposomal, or chemically modified agents. Of note, while the pacDNA exhibits an encouraging efficacy and safety profile, it may be desirable to have tunable degradability built into the bottlebrush polymer backbone as a means to control clearance. Thus, in some embodiments, the bottlebrush polymer backbone is degradable, e.g., tunably degradable. Towards this goal, in some embodiments, degradable materials may be adopted, including novel ring-opening metathesis polymerization (ROMP) polymers, condensation polymers with a non-aliphatic backbone, and/or miktoarm star polymers/nanoparticles, as long as the high-density PEG environment characteristic of the pacDNA is retained. In some embodiments, the PEGylated oligonucleotides and/or pacDNA are described in U.S. Pat. Nos. 10,590,414; 11,104,901; and US Patent Application No. 2018-0369142 (the contents of each of which is herein incorporated by reference in their entirety).

Thus, the present disclosure shows that the molecular brush enhances the delivery of conjugated ASOs in suppressing oncogenic KRAS in vivo, which massively reduces the dosage level required for a phenotypic response compared with naked ASOs. The pacDNA relaxes the requirement of ASO modification chemistry, which allows natural, unmodified nucleic acids to be used in place of chemically modified ASOs, bypassing their potential toxicity. The bottlebrush polymer also contributes significantly to the diminished clearance from systemic circulation and the enhanced tumor accumulation, while itself generating no apparent adverse toxic or immunogenic side effects. Collectively, the present disclosure results highlight the potential of pacDNA as an antisense agent that directly targets the highly unmet clinical need represented by cancers, e.g., KRAS-driven human cancers. Further, the general platform serves as a novel, single-entity alternative to current paradigms in oligonucleotide therapeutics, including modified oligonucleotides and formulations with liposomes/lipid nanoparticles.

As such, in some embodiments, the present disclosure provides for methods, systems and compositions comprising a PEG bottlebrush polymer-LNA conjugate that effectively inhibits the growth of a cancer, e.g., non-small cell lung cancer, e.g., in the NCI-H358 xenograft model, with significantly reduced dosage. Chemically modified ASOs with enhanced stability, after being combined with bottlebrush polymer, show prolonged blood circulation times and high retention levels at tumor sites. Those characteristics result in a reduced total dosage of pacLNA, ˜1% of previously reported studies. Therefore, in certain embodiments, the present disclosure provides for methods and compositions that leverage the side effects and toxicities of fully modified ASOs, and provide a safe and translatable platform for next-generations ASOs.

Further disclosed herein, in certain embodiments, the present disclosure provides for methods and compositions comprising pacDNA in the context of treating NSCLC harboring KRAS^{MUT} In still further embodiments, the disclosure provides for a library of pacDNA constructs having an identical ASO base sequence but with variation in ASO chemistry, releasability, and degree of steric shielding was tested. As described herein, the present disclosure reports the in vitro and in vivo pharmacological properties of materials, describes the dosage-dependent antitumor response in mice bearing KRAS^{MUT} NSCLC xenografts, and characterizes the safety profile of certain pacDNA in mice. Comparing an optimized pacDNA with a clinical ASO targeting the same transcript region (AZD4785), pacDNA achieved more pronounced tumor suppression levels than AZD4785 but at a fraction (2.5%) of the dosage and with reduced dosing frequency. In addition, the treatment was free of common deleterious side effects such as acute toxicity, inflammation, and immunogenic side effects. Overall, the pacDNA system provided by the present disclosure may offer a clinically viable approach to addressing KRAS-driven human cancers.

Also disclosed herein, the present disclosure provides for compositions and methods which incorporate LNA modifications of ASO with the bottlebrush polymer, e.g., to achieve high stability of pacLNA, and/or up to 8-week retention of PS pacLNA in tumor tissue after one single injection. These favorable biopharmaceutical properties of pacLNA maximize the efficacy and significantly lower the total dosage of chemically modified ASO-1% of the existing preclinical results of cEt-modified ASO.

### Methods and Compositions of the Disclosure

In one aspect, the present disclosure provides methods and compositions for inhibiting or reducing tumor and/or cancer growth or tumor size in a subject. In some embodiments, the method comprising the step of administering to the subject a composition comprising: a polyethylene glycol (PEG)-conjugated antisense oligonucleotide (ASO). In some embodiments, the methods and compositions disclosed herein provide for targeting a protein and/or gene and/or gene product in a subject. In some embodiments, the ASO targets an oncogene.

“Inhibition of growth” (e.g., referring to cancer cells, such as tumor cells) refers to a measurable decrease in the cell growth in vitro or in vivo when the cell is contacted with a drug or drugs, when compared to the growth of the same cell grown in appropriate control conditions well known to the skilled in the art. Inhibition of growth of a cell in vitro or in vivo may be at least about 10%, 20%, 30%, 40%, 50%, 60%, 70%, 80%, 90%, 99%, or 100%.

In still further embodiments, the methods and compositions provide that the oncogene is a RAS gene. In some embodiments, the RAS gene is KRAS, HRAS, or NRAS. In further embodiments, the RAS gene comprises at least one mutation. In still further embodiments, the ASO targets the oncogene 3′ UTR and/or the oncogene 5′ UTR.

In some embodiments, the PEG-conjugated ASO is a polymer-assisted compaction of DNA (pacDNA). In further embodiments, the pacDNA is a phosphorothioate (PS) pacDNA, a phosphodiester (PO) pacDNA, a PEG-conjugated locked nucleic acid (LNA)-pacLNA, or a combination thereof.

In some embodiments, the bottlebrush polymer-ASO conjugate comprises a chemically modified or unmodified ASO covalently linked to the backbone of the bottlebrush polymer. In still further embodiments, the bottlebrush polymer-ASO conjugate comprises a plurality of PEG side chains. In some embodiments, the bottlebrush polymer-ASO conjugate comprises at least about 5 to at least about 50 PEG side chains.

In still further embodiments, the pacDNA is a bottlebrush polymer-ASO conjugate comprising chemically modified or unmodified ASO covalently linked to the backbone of a bottlebrush polymer, having a multitude of PEG side chains (between 5-50). In still further embodiments, the PEG is a Y-shaped PEG.

In some embodiments of the disclosure, the ASO targets an oncogene mRNA 3′ UTR, coding region, or 5′ UTR. In some embodiments, the pacDNA comprises one ASO, two ASOs, or a plurality of ASOs, wherein the ASO comprises an anti-KRAS oligonucleotide. In some embodiments, the ASO or ASOs is/are natural. In further embodiments, the ASO or ASOs is/are chemically modified. In still further embodiments, the ASO or ASOs comprise a conjugation site. In some embodiments, the conjugation site is at a sequence terminus or in an internal position, or a combination thereof. In some embodiments, the ASO or ASOs further comprise sequences that affect releasability (e.g., rendering the ASO more or less stable, more or less bioreductively cleavable).

In some embodiments, the disclosure provides for methods of treatment and methods of enhancing efficacy of treatment of a disorder, e.g., cancer, comprising administration of the compositions described herein. In some embodiments, the disclosure provides for inhibiting initiation of cancer. In some embodiments, the disclosure provides for inhibiting maintenance and/or metastasis. In some embodiments, the methods and compositions reduce rapid cell growth and/or proliferation.

As used herein, “therapy,” “treat,” “treating,” or “treatment” means inhibiting or relieving a condition in a subject in need thereof. For example, a therapy or treatment refers to any of: (i) the prevention of symptoms associated with a disease or disorder (e.g., cancer); (ii) the postponement of development of the symptoms associated with a disease or disorder (e.g., cancer); and/or (iii) the reduction in the severity of such symptoms that will, or are expected, to develop with said disease or disorder (e.g., cancer). The terms include ameliorating or managing existing symptoms, preventing additional symptoms, and ameliorating or preventing the underlying causes of such symptoms. Thus, the terms denote that a beneficial result is being conferred on at least some of the subjects (e.g., humans) being treated. Many therapies or treatments are effective for some, but not all, subjects that undergo the therapy or treatment.

As used herein, the term “effective amount” means an amount of a composition, that when administered alone or in combination to a cell, tissue, or subject, is effective to achieve the desired therapy or treatment under the conditions of administration. For example, an effective amount is one that would be sufficient to produce an immune response to bring about effectiveness of a therapy (therapeutically effective) or treatment. The effectiveness of a therapy or treatment (e.g., eliciting a humoral and/or cellular immune response) can be determined by suitable methods known in the art.

As used herein, “subject” or “patient” includes humans, domestic animals, such as laboratory animals (e.g., dogs, monkeys, pigs, rats, mice, etc.), household pets (e.g., cats, dogs, rabbits, etc.) and livestock (e.g., chickens, pigs, cattle (e.g., a cow, bull, steer, or heifer), sheep, goats, horses, etc.), and non-domestic animals. In some embodiments, a subject is a mammal (e.g., a non-human mammal). In some embodiments, a subject is a human. In still further embodiments, a subject of the disclosure may be a cell, cell culture, tissue, organ, or organ system.

In some embodiments the subject is about 0-3 months, 0-6 months, 6-11 months, 12-15 months, 12-18 months, 19-23 months, 24 months, 1-2 years, 2-3 years, 4-6 years, 7-10 years, 11-12 years, 11-15 years, 16-18 years, 18-20 years, 20-25 years, 25-30 years, 30-35 years, 30-40 years, 35-40 years, 30-50 years, 30-60 years, 50-60 years, 60-70 years, 50-80 years, 70-80 years, 80-90 years, or older than 60 years.

In still further embodiments, the method comprises administering to the subject an effective amount of the composition, or a pharmaceutically acceptable salt thereof.

The term “pharmaceutically acceptable salts” embraces salts commonly used to form alkali metal salts and to form addition salts of free acids or free bases. The nature of the salt is not critical, provided that it is pharmaceutically acceptable.

Suitable pharmaceutically acceptable acid addition salts may be prepared from an inorganic acid or an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts.

Suitable pharmaceutically acceptable base addition salts include, but are not limited to, metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine. Pharmaceutically acceptable basic/cationic salts also include, the diethanolamine, ammonium, ethanolamine, piperazine and triethanolamine salts.

All of these salts may be prepared by conventional means by treating, for example, a composition described herein with an appropriate acid or base.

In some embodiments, compositions of the disclosure are administered in a delivery vehicle comprising a nanocarrier selected from the group consisting of a lipid, a polymer and a lipo-polymeric hybrid. In still further embodiments, the first and second polynucleotides are encapsulated in a lipid nanoparticle, polymer nanoparticle, virus-like particle, nanowire, exosome, or hybrid lipid/polymer nanoparticle. In some embodiments, the first and second polynucleotides are encapsulated in the same nanocarrier. In still further embodiments, the first and second polynucleotides are encapsulated in different nanocarriers. In some embodiments, the lipid nanoparticle is ionizable.

As used herein, the term “pharmaceutically acceptable” refers to species which are, within the scope of sound medical judgment, suitable for use without undue toxicity, irritation, allergic response and the like, and are commensurate with a reasonable benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

A desired dose may conveniently be administered in a single dose, for example, such that the agent is administered once per day, or as multiple doses administered at appropriate intervals, for example, such that the agent is administered 2, 3, 4, 5, 6 or more times per day. The daily dose can be divided, especially when relatively large amounts are administered, or as deemed appropriate, into several, for example 2, 3, 4, 5, 6 or more, administrations. Typically, the compositions will be administered from about 1 to about 6 (e.g., 1, 2, 3, 4, 5 or 6) times per day or, alternatively, as an infusion (e.g., a continuous infusion).

Determining the dosage and route of administration for a particular agent, patient and disease or condition is well within the abilities of one of skill in the art. Preferably, the dosage does not cause or produces minimal adverse side effects.

Doses lower or higher than those recited above may be required. Specific dosage and treatment regimens for any particular subject will depend upon a variety of factors, for example, the activity of the specific agent employed, the age, body weight, general health status, sex, diet, time of administration, rate of excretion, drug combination, the severity and course of the disease, condition or symptoms, the subject's disposition to the disease, condition or symptoms, the judgment of the treating physician and the severity of the particular disease being treated. The amount of an agent in a composition will also depend upon the particular agent in the composition.

In some embodiments, the concentration of one or more active agents provided in a composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, or 0.01% w/w, w/v or v/v; and/or greater than 90%, 80%, 70%, 60%, 50%, 40%, 30%, 20%, 10%, 5%, 1%, 0.5%, 0.4%, 0.3%, 0.2%, 0.1%, or 0.01% w/w, w/v, or v/v.

In some embodiments, the concentration of one or more active agents provided in a composition is in the range from about 0.01% to about 50%, about 0.01% to about 40%, about 0.01% to about 30%, about 0.05% to about 25%, about 0.1% to about 20%, about 0.15% to about 15%, or about 1% to about 10% w/w, w/v or v/v. In some embodiments, the concentration of one or more active agents provided in a composition is in the range from about 0.001% to about 10%, about 0.01% to about 5%, about 0.05% to about 2.5%, or about 0.1% to about 1% w/w, w/v or v/v.

In some embodiments, the present disclosure provides for a method of treatment for a cancer and/or a tumor. In some embodiments, the present disclosure provides for the treatment of a KRAS-mediated disease or disorder.

In some embodiments, the ASO has at least about 80% sequence identity to SEQ ID NO: 1, for example, at least about: 75%, 80%, 85%, 90%, 95%, 96%, 97%, 98%, 99% or 100% sequence identity to SEQ ID NO: 1. In some embodiments, the ASO comprises a sequence that has about 75%, 80%, 85%, 90%, 95%, 96%, 97%, 98%, 99% or 100% sequence identity to SEQ ID NO: 1.

As used herein, the term “sequence identity,” refers to the extent to which two sequences have the same residues at the same positions when the sequences are aligned to achieve a maximal level of identity, expressed as a percentage. For sequence alignment and comparison, typically one sequence is designated as a reference sequence, to which a test sequences are compared. Sequence identity between reference and test sequences is expressed as a percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide residue at 70% of the same positions over the entire length of the reference sequence.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Natl. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology). In some embodiments, codon-optimized sequences for efficient expression in different cells, tissues, and/or organisms reflect the pattern of codon usage in such cells, tissues, and/or organisms containing conservative (or non-conservative) amino acid substitutions that do not adversely affect normal activity.

In some embodiments, the ASO comprises a plurality ASOs, wherein the plurality of ASOs comprises anti-KRAS oligonucleotides of different nucleotide sequences.

In still further embodiments, the pacDNA comprises at least two anti-KRAS oligonucleotides and wherein the at least two anti-KRAS oligonucleotides comprise different nucleotide sequences. In some embodiments, the at least two anti-KRAS oligonucleotides comprises less than about 100% sequence identity.

In some embodiments, KRAS mRNA is reduced. As used herein, the term “reducing” or “reduce” refers to modulation that decreases risk (e.g., the level prior to or in an absence of modulation by the agent). In some embodiments, the agent (e.g., composition) reduces risk, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In certain embodiments, the agent (e.g., composition) decreases risk, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In particular embodiments, the agent (e.g., composition) decreases risk, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference.

In certain embodiments, the administration of the composition may be carried out in any manner, e.g., by parenteral or nonparenteral administration, including by aerosol inhalation, injection, infusions, ingestion, transfusion, implantation or transplantation. For example, the compositions described herein may be administered to a patient trans-arterially, intradermally, subcutaneously, intratumorally, intramedullary, intranodally, intramuscularly, by intravenous (i.v.) injection, intranasally, intrathecally or intraperitoneally. In one aspect, the compositions of the present disclosure are administered intravenously. In one aspect, the compositions of the present disclosure are administered to a subject by intramuscular or subcutaneous injection. The compositions may be injected, for instance, directly into a tumor, lymph node, tissue, organ, or site of infection.

In some embodiments, compositions as described herein are used in combination with other known agents and therapies, such as chemotherapy, transplantation, and radiotherapy. Administered “in combination”, as used herein, means that two (or more) different treatments are delivered to the subject during the course of the subject's treatment e.g., the two or more treatments are delivered after the subject has been diagnosed with the disease and before the disease has been cured or eliminated or treatment has ceased for other reasons. In some embodiments, different treatments (e.g., additional therapeutics) can be administered simultaneously or sequentially.

In some embodiments, the methods and compositions of the disclosure provide for a reduction in the minimum dosage administered to a subject in need thereof. Determining the dosage and route of administration for a particular agent, patient and disease or condition is well within the abilities of one of skill in the art. Preferably, the dosage does not cause or produces minimal adverse side effects.

Doses lower or higher than those recited above may be required. Specific dosage and treatment regimens for any particular subject will depend upon a variety of factors, for example, the activity of the specific agent employed, the age, body weight, general health status, sex, diet, time of administration, rate of excretion, drug combination, the severity and course of the disease, condition or symptoms, the subject's disposition to the disease, condition or symptoms, the judgment of the treating physician and the severity of the particular disease being treated. The amount of an agent in a composition will also depend upon the particular agent in the composition.

In some embodiments, the methods and compositions disclosed herein, provide that the rate of excretion of the PEG-conjugated ASO administered to the subject is reduced when compared to the rate of excretion of an ASO without the PEG-conjugate administered to a comparable subject. In some embodiments, the ASO bioactivity in the subject administered the PEG-conjugated ASO is greater than the ASO bioactivity of an ASO without a PEG-conjugate administered to a comparable subject.

As used herein, “comparable subject” means a subject of similar age, sex and/or other demographic parameters as the sample/subject to whom the therapy or treatment is administered.

In some embodiments, the methods and compositions are for use in treating cancer. In some embodiments, the cancer is non-small cell lung cancer, colorectal cancer, pancreatic cancer, or any combination thereof.

In some embodiments, the disclosure provides for a method of inhibiting or reducing tumor growth in a subject, said method comprising administering to the subject an effective amount of a pacDNA comprising a plurality (e.g., multitude) of anti-sense oligonucleotides (ASOs) that specifically binds an oncogene. In some aspects, the oncogene is the KRAS gene. In some aspects, the KRAS gene comprising at least one mutation. In some aspects, the pacDNA is a phosphorothioate (PS) pacDNA. In some aspects, the pacDNA is a phosphodiester (PO) pacDNA. In some aspects, the subject has non-small cell lung cancer (NSCLC). In some aspects, the ASOs are identical in nucleotide sequence. In some aspects, the plurality of ASOs comprises anti-KRAS oligonucleotides of different nucleotide sequences.

In some embodiments, the disclosure provides for an anti-sense oligonucleotide-loaded pacDNA comprising a plurality of anti-sense oligonucleotides (ASOs) specific for an oncogene coupled to a brush-polymer backbone, e.g., wherein the antisense (anti-sense) oligonucleotide specifically binds an oncogene. In some aspects, the oligonucleotide is specific for the KRAS gene. In some aspects, the KRAS gene comprises at least one mutation. In some aspects, the anti-sense oligonucleotide-loaded pacDNA is a phosphorothioate (PS) pacDNA. In some embodiments, the pacDNA is a phosphodiester (PO) pacDNA. In some aspects, the ASOs are identical in nucleotide sequence. In some aspects, the plurality of ASOs comprises anti-KRAS oligonucleotides of different nucleotide sequences.

### Exemplification

### Materials and Methods (Examples 1-4)

Oligonucleotide synthesis. Oligonucleotides (both PO and PS versions) were synthesized on a Model 391 DNA synthesizer (Applied Biosystems, Inc., CA, USA) using standard solid-phase phosphoramidite methodology. DNA strands were cleaved from the CPG support using ammonium hydroxide (28% NH₃ in H₂O) at room temperature for 24 h and purified by reverse-phase HPLC liquid chromatography. The dimethoxytrityl (DMT) protecting group was removed by treatment with 20% acetic acid in H₂O for 1 h, followed by extraction with ethyl acetate three times. Upon purification, DNA was stored at −20° C. To synthesize the dye-labeled DNA, 3′-(6-fluoresecein) CPG, Cy3 CPG, and cyanine 5 (Cy5) CPG were used to synthesize the antisense strands. 5′ dibenzocyclooctyl (DBCO) groups were incorporated by using 5′-DBCO-TEG phosphoramidite. To synthesize DBCO-SS-DNA, purified 5′ amine-modified DNA (100 nmol) was dissolved in 100 μL of NaHCO₃ (0.1 M) buffer, to which 0.5 mg dibenzocyclooctyne-SS—N-hydroxysuccinimidyl ester (DBCO-SS-NHS) was added via 100 μL DMSO solution. The reaction mixture was shaken at 0° C. overnight. The products (DBCO-SS-DNA) were purified by reverse-phase HPLC. To install mid-sequence DBCO groups, amine-modified DNA strands were first synthesized using an amino modifier (amine-C6 dG), which were then reacted with dibenzocyclooctyne-N-hydroxysuccinimidyl (DBCO-NHS) or dibenzocyclooctyne-SS—N-hydroxysuccinimidyl ester (DBCO-SS-NHS) in 0.1 M bicarbonate solution overnight at 4° C. The reaction mixture was passed through a NAP-10 column (G.E. Health) and then purified using the reverse-phase HPLC. The successful syntheses of all oligonucleotides were confirmed by MALDI-TOF MS.

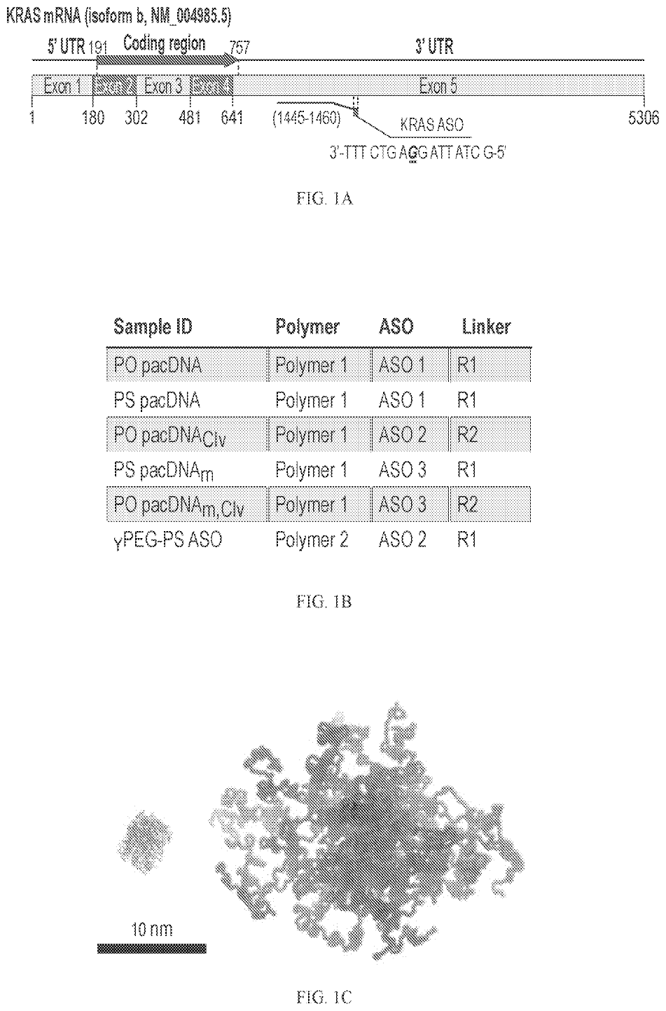

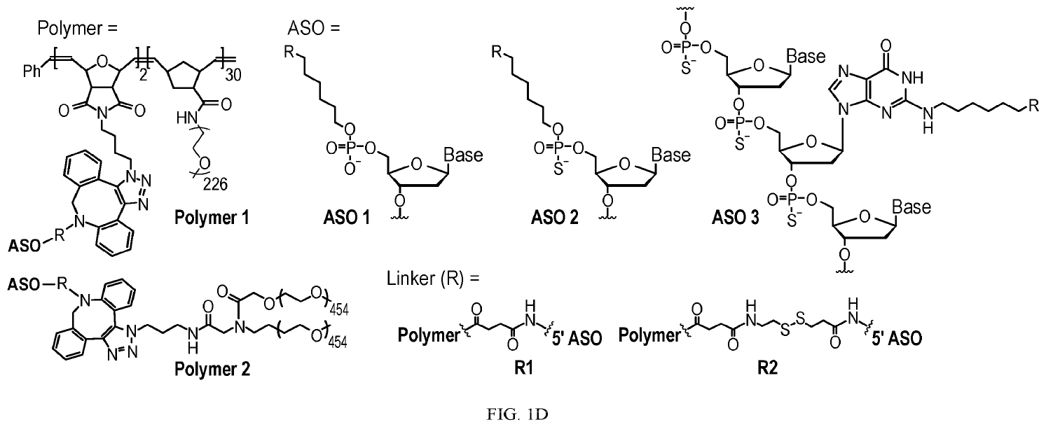

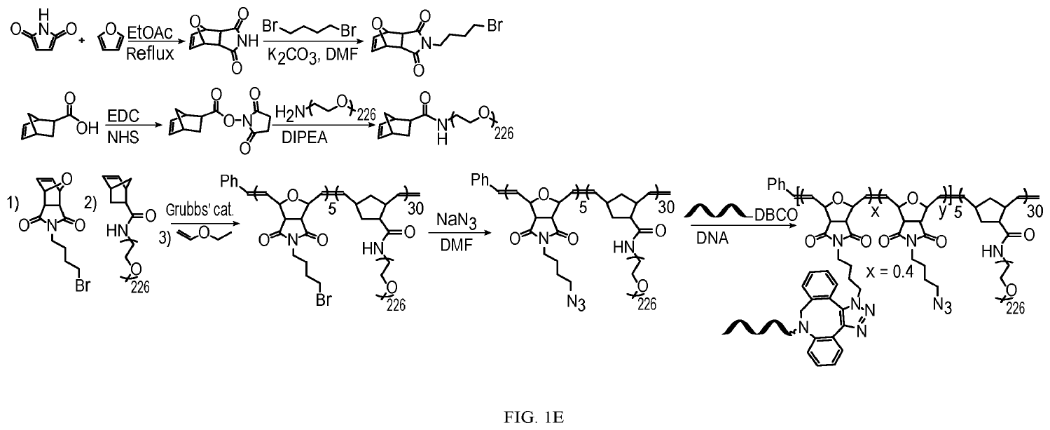

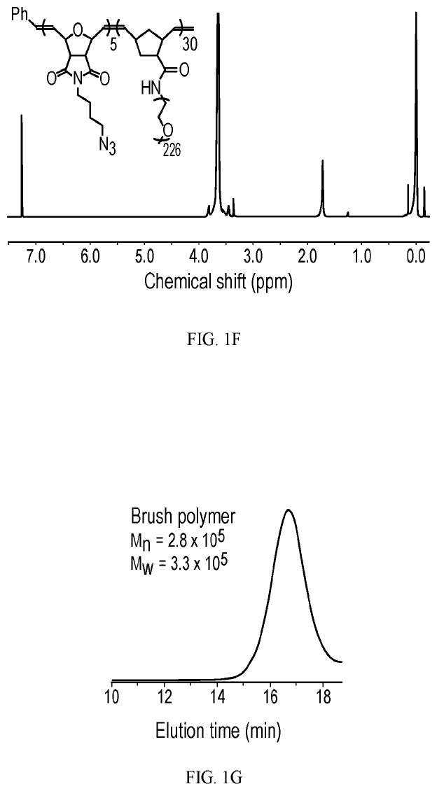

Synthesis of azide-functionalized bottlebrush polymer. Two monomers, norbornenyl bromide and norbornenyl PEG, were synthesized following procedures described in Lu, X., et al. (Journal of the American Chemical Society, 137 (39), 12466-12469; herein incorporated by reference in its entirety)). Modified 2nd generation Grubbs catalyst was prepared based on a published method shortly prior to use (Love, J. A., et al. (Angewandte Chemie, 114 (21), 4207-4209; herein incorporated by reference in its entirety)). Next, norbornenyl bromide (5 equiv.) was dissolved in deoxygenated dichloromethane under N₂ and cooled to −20° C. in an ice-salt bath. The modified Grubbs' catalyst (1 equiv.) in deoxygenated dichloromethane was added to the solution via a gastight syringe, and the solution was stirred vigorously for 30 min. After thin-layer chromatography (TLC) confirmed the complete consumption of the monomer, norbornenyl PEG (50 equiv.) in deoxygenated dichloromethane was added to the reaction, and the mixture was stirred for 6 h. Several drops of ethyl vinyl ether (EVE) were added to quench the reaction and the solution was stirred for an additional 2 h. After concentration under vacuum, the residue was precipitated into cold diethyl ether three times. The precipitant was dried under vacuum to afford a dry powder. Subsequently, the resulting brush polymer was treated with an excess of sodium azide in anhydrous N,N-dimethylformamide (DMF) overnight at room temperature. The materials were transferred to a dialysis tubing (MWCO, 10 kDa), dialyzed against Nanopure™ water for 24 h, and lyophilized to afford a white, dry powder. The successful incorporation of azide functionalities was confirmed via FT-IR. The number of azide groups per copolymer available for coupling was estimated by reacting with alkyne-modified fluorescein and subsequent comparison of the fluorescence with a standard curve established with free fluorescein. The final polymer was characterized by ¹H nuclear magnetic resonance (NMR) and N,N-dimethylformamide (DMF) GPC (FIGS. 1F and 1G).

Synthesis of azide-functionalized Y-shape PEG. Y-shaped PEG NHS ester (1 equiv.), 3-azido-1-propanamine (2 equiv.), and N, N-diisopropylethylamine (2 equiv.) were dissolved in anhydrous dichloromethane and added to a round bottom flask. The reaction mixture was stirred overnight at room temperature and precipitated into diethyl ether three times. The product was purified by a NAP-10 column and lyophilized as a white powder with a recovery yield of 80%.

Synthesis of Cy5-labeled bottlebrush polymer. The bottlebrush polymer was labeled with Cy5 via copper-catalyzed click chemistry for in vivo fluorescence tracking. The polymer (30 mg, 100 nmol) in Nanopure™ water (3 mL) was added with Cy5-alkyne (110 nmol, 110 μL 1 mM DMSO solution). The catalyst system (CuSO₄·5H₂O, 80 nmol; tris-hydroxypropyltriazolylmethylamine [THPTA], 100 nmol; sodium ascorbate, 500 nmol) was added to the solution and stirred at room temperature for 12 h. The reaction mixture was dialyzed against Nanopure™ water and further purified using aqueous GPC. The fractions containing the conjugate were collected, concentrated, desalted, and lyophilized to afford a blue powder. UV-Vis spectroscopy indicates that there was ˜1.0 Cy5 dye molecule per polymer.

Synthesis of pacDNAs. In a typical procedure, azide-functionalized brush copolymers (15 mg, 50 nmol) were dissolved in 500 μL aqueous NaCl solution (2 M), to which DBCO-modified DNA (100 nmol) in 200 μL aqueous NaCl solution (2 M) was added (2 equiv. to N₃). The reaction mixtures were shaken gently for 24 h at 50° C. on an Eppendorf Thermomixer. The conjugates were purified using aqueous GPC to remove the unreacted DNA. Thereafter, the collected fractions were concentrated, desalted with a NAP-25 column, and lyophilized to yield a white powder (or green/red/blue powders for fluorescein-, Cy3-, and Cy5-labeled conjugates, respectively). To synthesize _{Y}PEG-DNA conjugate, 2 mg (50 nmol) of Y-shaped PEG-azide was mixed with 60 nmol DBCO-modified DNA strands in 200 μL aqueous NaCl solution (2 M). The reaction mixture was shaken at 50° C. for 24 h and purified by reverse-phase HPLC. After purification, the conjugate was desalted by a NAP-10 column and lyophilized to yield a white powder.

Molecular dynamics (MD) simulation. MARTINI coarse-grained (CG) force-field was used for MD simulation of pacDNA in explicit solvation by water and neutralizing sodium ions (Marrink, S. J., et al. (The journal of physical chemistry B, 111 (27), 7812-7824.55; herein incorporated by reference in its entirety)). The force field incorporates four heavy atoms with similar chemical identities into one CG bead, and therefore reduces the freedoms of the molecules needed to calculate. Bonded parameters are defined based upon molecular structure, while non-bonded parameters, including van der Waals and electrostatic forces, are derived from free energy partitioning between polar and organic solvents. The MARTINI version of PEG was developed by Lee, H., et al. (The journal of physical chemistry B, 113 (40), 13186-13194; herein incorporated by reference in its entirety). The atomistic to CG mapping is 3:1 for the PEG monomer. This mapping ratio deviates from the standard MARTINI mapping scheme due to the size of the PEG monomer. Herein, the PEG monomer is represented by an SN0 particle in the CG force field. The parameters for the Lennard-Jones interaction between PEG and water are σ=0.47 nm and ε=4.0 KJ/mol. The time step of CG MD simulations was set to be 0.010 ps. Periodic boundaries conditions were used in all directions. The system was controlled using an NPT ensemble. The temperature was controlled at 310 K using the Berendsen thermostat while the pressure was controlled at 1 atm using the Berendsen barostat (Berendsen, H. J., et al. (The Journal of chemical physics, 81 (8), 3684-3690; herein incorporated by reference in its entirety)). The cutoff distances of van der Waals and short-range electrostatic interactions were set at 1.2 nm. Long-range electrostatic interactions were not considered. All simulations were performed using the GROMACS 2018 package (Van Der Spoel, D., et al. (Journal of computational chemistry, 26 (16), 1701-1718; herein incorporated by reference in its entirety)).

Hybridization and nuclease degradation kinetics. For hybridization kinetics, fluorescein-labeled pacDNA and controls were dissolved in PBS buffer (pH 7.4) at a final DNA concentration of 100 nM. Each sample (1 mL) was transferred to a fluorescence cuvette, to which dabcyl-labeled complementary strand or non-complementary dummy strands (2 equiv.) were added via 2 μL of PBS solution. The solution was rapidly mixed with a pipette. The fluorescence of the solution (ex=494 nm, em=522 nm) was continuously monitored before the mixing and every 3 sec thereafter using a Cary Eclipse fluorescence spectrometer. The endpoint was determined by adding a large excess (10 equiv.) of the complementary dabcyl-DNA to the mixture, followed by incubation for 2 h. The kinetics plots were normalized to the endpoint determined for each sample, and the reported values are the average of three independent experiments.

For nuclease degradation, pacDNA and controls (1 μM DNA basis; fluorescein-labeled) were each mixed with their complementary dabcyl-labeled DNA (2 μM) in PBS buffer. The solutions were gently shaken at room temperature overnight. Subsequently, 100 μL of each sample was withdrawn and diluted to 100 nM with assay buffer (50 mM tris-HCl, 50 mM NaCl, and 20 mM MnCl₂, pH=7.5), to which DNase I (0.1 unit/mL) was added and rapidly mixed. The fluorescence of each sample was monitored before the addition of DNase I and every 3 seconds thereafter (ex=494 nm, em=522 nm) for 10 h. The endpoint of each sample was determined by measuring the fluorescence of pacDNAs or controls at an identical concentration in the absence of the dabcyl-labeled complementary strand. The kinetics plots were normalized to the endpoints of each sample, and the reported values are the average of three independent experiments.

DNA release in vitro. Conjugates (PS pacDNA, PS pacDNAₘ, PS pacDNA_{Clv} and PS pacDNA_{m,Clv}, 100 nM) were mixed with 10 mM dithiothreitol (DTT) in 1× PBS at 37° C. for 1 h. Thereafter, the solutions were subject to agarose gel electrophoresis using 1% agarose gel in 0.5× TBE buffer with a running voltage of 120 V. The amount of DNA released was determined using band densitometry analysis. The experiment was conducted in triplicate.

Cell culture, flow cytometry, and confocal microscopy. Cells were cultured in RPMI 1640 supplied with 10% fetal bovine serum (FBS), 1% L-glutamine, and 1% antibiotics at 37° C. in a humidified atmosphere containing 5% CO₂. Cellular uptake of pacDNAs and controls was evaluated using flow cytometry and confocal laser scanning microscopy (CLSM). For flow cytometry, cells were seeded in 24-well plates at a density of 2.0×10⁵ cells per well in 1 mL full growth medium and cultured for 24 h at 37° C. with 5% CO₂. After washing by PBS 2×, Cy3-labeled pacDNAs and controls (250 nM-5 μM equiv. of ASO) dissolved in RPMI culture medium (either serum-free or with 10% FBS) was added, and cells were further incubated at 37° C. for 4 h. Subsequently, cells were washed with PBS 3× and suspended by treatment with trypsin. Thereafter, 2 mL of PBS was added to each culture well, and the solutions were centrifugated for 5 min (1000 rpm). Cells were then resuspended in 0.5 mL of PBS for flow cytometry analysis on a BD FACS Calibur flow cytometer. Data for 1.0×10⁴ gated events were collected.

For confocal microscopy, cells were seeded in 24-well glass bottom plates at a density of 1.0×10⁵ cells per well and cultured in 1 mL complete culture medium for 24 h at 37° C. After washing by PBS 2×, Cy3-labeled pacDNAs and controls (250 nM-5 μM equiv. of ASO) dissolved in RPMI culture medium (either serum-free or with 10% FBS) was added, and cells were further incubated at 37° C. for 4 h. Thereafter, cells were washed with PBS 3× and fixed with 4% paraformaldehyde for 30 min at room temperature, followed by another 3× washing with PBS. The cells were then stained with Hoechst 33342 for 10 min and imaged on an LSM-700 confocal laser scanning microscope (Carl Zeiss Ltd., Cambridge, UK). Imaging settings were kept identical for all samples in each study.

Pharmacological inhibition of cellular uptake. To study the cellular internalization pathway, NCI-H358 cells (2.0×10⁵) were seeded into 24-well plates and incubated at 37° C. overnight for cells to settle down. The cells were pretreated with rottlerin (1 or 3 μg/mL), methyl-β-cyclodextrin (MβCD, 2.5 or 12.5 mg/mL), chloropromazine (CPM, 1 or 5 μg/mL) or sodium azide (NaN₃, 10 or 50 mM) for 30 min, before being further incubated with 2 μM Cy3-labeled pacDNAs or free PS ASO for 4 h. The inhibitor concentrations were maintained in the cell culture medium throughout the experiments. Thereafter, the cells were washed with PBS 3× and harvested by trypsinization. All samples were analyzed by flow cytometry (FACS Calibur, BD Bioscience, San Jose, CA) to determine the extent of cellular internalization. All measurements were performed in triplicate and the results were averaged.

MTT cytotoxicity assay. The cytotoxicity of free ASOs, bottlebrush polymer, and pacDNAs was evaluated with the MTT (dimethylthiazol-diphenyltetrazolium bromide) colorimetric assay for NCI-H358, NCI-H1944, and PC9 cells. Briefly, 1.0×10⁴ cells were seeded into 96-well plates in 200 μL DMEM per well and were cultured for 24 h. The cells were then treated with pacDNAs and controls at varying concentrations of ASO or polymer (0.25 through 10 μM; ASO basis). Cells treated with vehicle (PBS) were set as a negative control. After 48 h of incubation, 20 μL of 5 mg/mL MTT stock solution in PBS was added to each well. The cells were incubated for another 4 h, and the medium containing unreacted MTT was removed carefully. The resulting blue formazan crystals were dissolved in DMSO (200 μL per well), and the absorbances (490 nm) were measured on a BioTek® Synergy™ Neo2 Multi-Mode microplate reader (BioTek Inc., VT, USA).

Hemolytic activity assay. A hemoglobin-free red blood cell (RBC, 2% w/v) suspension was prepared by repeated centrifugation (2000 rpm for 10 min at 4° C.) and resuspension in ice-cold PBS for a total of 3×. After the final resuspension, the concentration of RBCs was adjusted to 2% w/v. Thereafter, samples and controls were dissolved in PBS, added to the RBC suspension in 1:1 (v:v) ratio, and incubated for 1 h at 37° C. Complete hemolysis was attained using 2% v/v Triton-X, yielding the 100% control value. After incubation, centrifugation (2000 rpm for 10 min at 4° C.) was used to isolate intact RBCs, and the supernatants containing released hemoglobin were transferred to quartz cuvettes for spectrophotometric analysis at 545 nm. Results were expressed as the amount of hemoglobin released as a percentage of total. All measurements were performed in triplicate and the results were averaged.

Western blot analysis. Cells (NCI-H358, NCI-H1944, or PC9) were plated at a density of 2.0×10⁵ cells per well in 24-well plates in RPMI medium and cultured overnight at 37° C. with 5% CO₂. Thereafter, samples and controls (1-10 μM equiv. ASO) in serum-free media were added to the wells and incubated with the cells for 4 h, before serum was added to the incubation mixture. Cells were cultured for another 68 h. Thereafter, cells were harvested and whole cell lysates were collected in 100 μL of RIPA Cell Lysis Buffer with 1 mM phenylmethanesulfonylfluoride (PMSF, Cell Signaling Technology, Inc., MA, USA) following manufacturer's protocol. Protein content in the extracts was quantified using a bicinchoninic acid (BCA) protein assay kit (ThermoFisher, MA, USA). Equal amounts of proteins (30 μg/lane) were separated on 4-20% gradient SDS-PAGE and electro-transferred to nitrocellulose membrane. The membranes were then blocked with 3% BSA (bovine serum albumin) in TBST (Tris-buffered saline supplemented with 0.05% Tween-20) and further incubated with appropriate primary antibodies overnight at 4° C. After washing and incubation with secondary antibodies, detected proteins were visualized by chemiluminescence using the ECL Western Blotting Substrate (Thermo Scientific, USA). Antibodies used for Western blots were: KRAS antibody (cat. NBP2-45536; Novus Biologicals), β-actin (cat. AM4302), vinculin clone hVIN-1 (cat. V9131; Sigma Aldrich), phospho-ERK1/2 clone E10 (T202/Y2014; cat. 9106), phospho-MEK1/2 clone 41G9 (S218/S222; cat. 9154), caspase 3 (cat. 9668), anti-rabbit IgG, HRP-linked antibody (cat. 7074P2), anti-mouse IgG, HRP-linked antibody (cat. 7076S). Unless otherwise noted, antibodies were obtained from Cell Signaling Technologies. Western blot images were quantified using the ImageJ software by comparing the detected protein band with that of the housekeeping protein.

Flow cytometric analysis of apoptosis. Cells were plated at a density of 2.0×10⁵ cells per well in 24-well plates in RPMI medium and cultured overnight at 37° C. with 5% CO₂. Thereafter, samples and controls (10 μM equiv. ASO) in culture media were added to the wells and incubated with the cells for 48 h. Apoptotic cells were determined using annexin V-fluorescein isothiocyanate (FITC)/propidium iodide (PI) apoptosis staining kit according to the manufacturer's instructions (cat. KA3805; Abnova™). Data were acquired using a FACS Calibur (BD Biosciences). All experiments were performed independently three times.

Animal studies. All mouse studies were approved by the Institutional Animal Care and Use Committee of Northeastern University and carried out under pathogen-free conditions in the animal facility of Northeastern University and in accordance with National Institutes of Health animal care guidelines. The animals had free access to sterile food pellets and water and were kept in the laboratory animal facility with temperature and relative humidity maintained at 23±2° C. and 50±20%, respectively, under a 12-h light/dark cycles. Mice were kept for at least 1 week to acclimatize them to the food and environment of the animal facility prior to experiments.

Plasma pharmacokinetics (PK). Immunocompetent mice (C57BL/6) were used to examine the plasma PK of free ASO (both PS and PO), Y-shaped PEG (40 kDa)-ASO conjugate, pacDNAs, and free bottlebrush polymer lacking an ASO component. Mice were randomly divided into nine groups (n=4). Cy5-labeled samples were i.v. administrated via the tail vein at equal ASO dosage (0.5 μmol/kg; free polymer concentration equals that of the pacDNAs). Of note, the fluorescence label is located on the ASO component except for the free polymer. Blood samples (25 μL) were collected from the submandibular vein at varying time points (30 min, 2 h, 4 h, 10 h, 24 h, 48 h and 72 h) using BD Vacutainer™ blood collection tubes with lithium heparin. Heparinized plasma was obtained by centrifugation at 3000 rpm for 15 min, aliquoted into a 96-well plate, and measured for fluorescence intensity on a BioTek® Synergy HT plate reader (BioTek Instruments Inc., VT, USA). The amounts of ASO in the blood samples were estimated using standard curves established for each sample. To establish the standard curves, samples of known quantities were incubated with freshly collected plasma for 1 h at room temperature before fluorescence was measured.

NCI-H358 xenograft tumor model preparation. To establish the NCI-H358 xenograft tumor model, approximately 4×10⁶ cells in 100 μL PBS were implanted subcutaneously on the right flank of 6-week-old BALB/c nude mice. Mice were monitored for tumor growth every other day.

Whole-animal and ex vivo organ imaging. NCI-H358 xenograft-bearing BALB/c nude mice were i.v. injected with Cy5-labeled samples at an ASO dose of 0.5 μmol/kg animal weight, and were scanned at 1, 4, 8, 24 h, and daily thereafter until 13 weeks or until fluorescence is no longer observable using an IVIS Lumina II imaging system (Caliper Life Sciences, Inc. MA, USA). To evaluate the biodistribution of pacDNAs and the bottlebrush polymer, mice were euthanized using CO₂, and major organs and the tumor were removed for biodistribution analysis. For the analysis of tumor penetration depth, tumors were immediately frozen in O.C.T compound (Fisher Scientific Inc., USA) 24 h after injection. The frozen tumor tissues were cut into 8 μm-thick sections using a cryostat, stained with Hoechst 33342, and imaged on an LSM-880 confocal laser scanning microscope (Carl Zeiss Ltd., Cambridge, UK).

Antitumor efficacy in NCI-H358 xenograft-bearing mice. To screen the pacDNA variants in antitumor efficacy, an NCI-H358 subcutaneous xenograft model was first established. When the xenograft reached a volume of ca. 100 mm³, mice were randomly divided into twelve groups (n=5) to receive the following via the tail vein: (1) PBS; (2) PO pacDNA (0.1 μmol/kg); (3) PS pacDNA (0.1 μmol/kg); (4) PS pacDNAₘ (0.1 μmol/kg); (5) free PS ASO (0.5 μmol/kg); (6) PO pacDNA (0.5 μmol/kg); (7) PS pacDNA (0.5 μmol/kg); (8) PS pacDNA_{Clv} (0.5 μmol/kg); (9) PS pacDNAₘ (0.5 μmol/kg); (10) PS pacDNA_{m,Clv} (0.5 μmol/kg); (11) scramble PS pacDNA (0.5 μmol/kg); (12) free bottlebrush polymer (0.5 μmol/kg). Samples were injected once every 3 days until day 36. The volume of tumors and weight of mice were recorded before every treatment and 3 days after the last treatment. Antitumor activity was evaluated in terms of tumor size by measuring two orthogonal diameters at various time points (V=0.5×ab²; a: long diameter, b, short diameter). At day 36, mice were euthanized with CO₂, and tumors and major organs (heart, lung, liver, spleen, and kidney) from each group were excised, fixed in 4% paraformaldehyde/PBS for 6 h, and placed into a 30% sucrose/PBS solution overnight at 4° C. The fixed tissues were paraffin-embedded and cut into 8 μm-thick sections with a cryostat. The sections were then processed with H&E staining. Immunohistochemistry staining of KRAS was carried out using mouse anti-KRAS primary antibody (1:1000 dilution, Invitrogen Co., CA, USA) and goat anti-mouse secondary antibody (1:5000 dilution, ThermoFisher, MA, USA).

Antitumor efficacy in NCI-H1944 xenograft-bearing mice. 8-week-old male athymic nude mice (n=5) were injected subcutaneously with ca. 5×10⁶ NCI-H1944 cells in 100 μL PBS on the right flank. When the mean tumor volume reached approximately 100 mm³, the tumor-bearing animals were treated i.v. with PBS, PO pacDNA, or PS pacDNA at 2.0 μmol/kg animal weight via the tail vein once every 3 days for 27 days. The volume of tumors and weight of mice were recorded before every treatment and on the third day after the last treatment. After that, the animals were euthanized by CO₂, and tumor samples were collected for immunohistochemical analysis. Main organs (lung, heart, liver, kidney, and spleen) were collected to assess toxicity through histological analysis.

Blood biochemistry. Healthy C57BL/6 mice (6-8 weeks, n=4) were injected i.v. with PO pacDNA, PS pacDNA, PS ASO, and free bottlebrush polymer three times a week for two weeks with the equal DNA or brush polymer dose of 0.5 μmol/kg animal weight. Blood samples were collected from the submandibular vein 24 h after the last injection, allowed to clot by being left undisturbed for 30 min, and centrifuged at 3000 rpm for 5 min, and the serum was collected. Serum aspartate aminotransaminase (AST), alanine aminotransferase (ALT), total bilirubin, albumin, total protein, and alkaline phosphatase (ALP) were measured as markers of hepatocellular and biliary injury. Blood urea nitrogen (BUN) and creatinine (CREA) were detected as renal function indexes. The measurements were performed by the Comparative Pathology Laboratory of MIT Division of Comparative Medicine.

Innate immune response. To evaluate potential innate immune responses to systemically delivered pacDNAs, immunocompetent C57BL/6 mice (n=4) were injected i.v. with samples and controls at an equal ASO concentration (0.5 μmol/kg; free polymer concentration equals that of the pacDNA). LPS (15 μg per animal) was used as a positive control. Two hours post-injection, serum samples were collected and processed to measure the representative cytokines (IL-1α, IL-1β, IL-4, IL-6, IL-10, IL-12 (p70), IFN-γ, and TNF-α) using enzyme-linked immunosorbent assay (ELISA) kits according to the manufacturer's protocol (Bio-Plex Mouse Cytokine Group I 8-plex Assay-Z6000004JP, Bio-Rad Laboratories, Inc., CA, USA).

Adaptive anti-PEG immunity and accelerated blood clearance. Healthy C57BL/6 mice (6-8 weeks, n=4) were administered Cy5-labeled PO pacDNA, PS pacDNA, and free bottlebrush polymer via the tail vein on days 1, 4, 11, and 25 at a dosage of 0.5 μmol/kg (ASO-basis; free polymer concentration equals that of the pacDNAs). Blood samples (25 L) were collected from the submandibular vein at preselected post-injection time points (0 min, 30 min, 4 h, 8 h, and 24 h). The concentration of circulating anti-PEG IgM and IgG antibodies was assessed by ELISA (Mouse Anti-PEG IgM ELISA and Mouse Anti-PEG IgG ELISA, Life Diagnostics Inc., PA, USA), according to the manufacturer's protocol. PK parameters were calculated using the similar method mentioned above.

To study the generation of anti-PEG immunoglobins following frequent exposures to pacDNA or conventional linear PEG-ASO conjugate in the blood, male C57BL/6 mice in groups of five were i.v. injected with pacDNAs (PO and PS), bottlebrush polymer, or _{Y}PEG-PS ASO at a dosage of 0.5 μmol/kg once every 3 days for 36 days (12 injections total). The serum of mice was collected on the 7^{th} and the 14^{th} day after the last injection, and the concentrations of circulating anti-PEG IgM and IgG antibodies were assessed by ELISA.

Statistics. All in vitro experiments were repeated at least three times. Statistical analysis was performed using GraphPad Prism 9. Data are presented as mean±standard deviation. Statistical methods used are indicated in the figure legends. Statistical significance was set at *p<0.05, **p<0.01, ***p<0.001, or ****p<0.0001.

### Materials (Example 5)

ω-Amine polyethylene glycol (PEG) methyl ether (Mₙ=10 kDa, PDI=1.05) was purchased from JenKem Technology (USA). Phosphoramidites and supplies for DNA synthesis were purchased from Glen Research Co. (Sterling, VA, USA). Human NCI-H358 lung cancer cell line was purchased from American Type Culture Collection (Rockville, MD, USA). All other materials were purchased from Fisher Scientific Inc. (USA), Sigma-Aldrich Co. (USA), or VWR International LLC. (USA) and used as received unless otherwise indicated.

### Methods (Example 5)

¹H nuclear magnetic resonance (NMR) spectra were recorded on a Varian 500 MHz NMR spectrometer (Varian Inc., CA, USA). MALDI-TOF mass spectrometry (MS) measurements were performed on a Bruker Microflex LT mass spectrometer (Bruker Daltonics Inc., MA, USA). Concentrations of samples were determined using a Nanodrop™ 2000 spectrophotometer (Thermo Scientific, USA). DLS and ζ potential measurements were performed on a Malvern Zetasizer Nano-ZSP (Malvern, UK). Samples were dissolved in Nanopure™ water at a concentration of 1 μM and filtered through a 0.2 μm PTFE filter before measurement. Fluorescence spectroscopy was carried out on a Cary Eclipse fluorescence spectrophotometer (Varian Inc., CA, USA). Reversed-phase high-performance liquid chromatography (RP-HPLC) was performed on a Waters (Waters Co., MA, USA) Breeze 2 HPLC system coupled to a Symmetry® C18 3.5 μm, 4.6×75 mm reversed-phase column and a 2998 PDA detector, using TEAA buffer (0.1 M) and HPLC-grade acetonitrile as mobile phases. Aqueous gel permeation chromatography (GPC) analysis was carried out on a Waters Breeze 2 GPC system equipped with a series of an Ultrahydrogel™ 1000, 7.8×300 mm column and three Ultrahydrogel™ 250, 7.8×300 mm columns and a 2998 PDA detector. Sodium nitrate solution (0.1 M) was used as the eluent running at a flow rate of 0.8 mL/min. N,N-dimethylformamide (DMF) GPC was performed on a Tosoh EcoSEC HLC-8320 GPC system (Tokyo, Japan) equipped with a TSKGel α-M 7.8×300 mm, 13 μm column and RI/UV-Vis detectors. HPLC-grade DMF with 0.05 M lithium bromide was used as the mobile phase, and samples were analyzed at a flow rate of 0.4 mL/min. DMF-GPC calibration was based on a ReadyCal kit of polyethylene glycol (PEG) standards (PSS-Polymer Standard Service-USA Inc., MA, USA). The kit covers an Mₙ range from 232 Da to 1015 kDa. For transmission electron microscopy (TEM), samples (10 μM) were placed on parafilm as a droplet, onto which a copper-coated TEM grid was gently placed. The grids were then moved, dried, and stained using 2% uranyl acetate for 10 min. TEM images were collected on a JEOL JEM 1010 electron microscope with an accelerating voltage of 80 kV.

Oligonucleotides Synthesis. All the LNA and DNA oligonucleotides were synthesized on a Dr. Oligo 48 (Biolytic, CA, USA) using standard solid-phase phosphoramidite methodology. Oligonucleotides were cleaved from the CPG support using ammonium hydroxide solution (28% NH₃ in H₂O) at room temperature for at least 17 h and purified via RP-HPLC. Then the dimethoxytrityl (DMT) protecting groups on the oligonucleotides were removed by treating with 20% acetic acid in H₂O for 1 h and extracted with ethyl ether 3×. Oligonucleotides were lyophilized and stored at −20° C. Dye-labeled oligonucleotides were synthesized on 3′-(6-fluoresecein) CPG, cyanine 3 (Cy3) CPG or cyanine 5 (Cy5) CPG. 5′ dibenzocyclooctyl (DBCO) groups were incorporated using 5′-DBCO-TEG phosphoramidite.

### Synthesis of pacLNAs

Norbornenyl bromide and norbornenyl PEG were synthesized as previously described in Pontrello, J. K., et al. (Journal of the American Chemical Society, 127 (42), 14536-14537; herein incorporated by reference in its entirety) and Lu, X., et al. (Journal of the American Chemical Society, 138 (29), 9097-9100; herein incorporated by reference in its entirety). Modified 2nd generation Grubbs catalyst was prepared based on a published method shortly prior to use (Love, J. A., et al. (Angewandte Chemie, 114 (21), 4207-4209); herein incorporated by reference in its entirety).

Next, norbornenyl bromide (5 equiv.) was dissolved in deoxygenated dichloromethane (DCM) under N₂ and cooled to −20° C. in an ice-salt bath. The modified Grubbs' catalyst (1 equiv.) in deoxygenated DCM was added to the solution via a gastight syringe, and the solution was stirred vigorously for 30 min. After thin-layer chromatography (TLC) confirmed the complete consumption of the monomer, norbornenyl PEG (50 equiv.) in deoxygenated DCM was added to the reaction, and the mixture was stirred for 6 h. Several drops of ethyl vinyl ether were added to quench the reaction and the solution was stirred for an additional 2 h. After concentration under vacuum, the residue was precipitated into cold diethyl ether 3×. The precipitant was dried under vacuum to afford a white powder. Subsequently, the brush polymer was reacted with an excess of sodium azide in anhydrous N,N-dimethylformamide (DMF) overnight at room temperature. The materials were transferred to a dialysis tubing (MWCO, 10 kDa), dialyzed against Nanopure™ water for 24 h, and lyophilized to afford a white powder. The azide-functionalized bottlebrush polymer (50 nmol) was dissolved in 1 mL sodium chloride solution (3 M) and reacted with DBCO-modified LNA oligonucleotides (100 nmol) at 50° C. overnight. The conjugate was purified by aqueous GPC, desalted, and lyophilized. The purified pacLNA were stored at −20° C. before use.

Synthesis of Cy5-labeled bottlebrush polymer. To label the bottlebrush polymer with Cy5, azide-functionalized bottlebrush polymer (100 nmol) and DBCO-modified sulfo-Cy5 (110 nmol) were dissolved in 3 M sodium chloride solution and shaken at 50° C. overnight. The reaction mixture was purified via aqueous GPC. The Cy5-labeled bottlebrush was collected and lyophilized to afford blue powder.

Hybridization kinetics. LNAs and pacLNAs were dissolved in PBS (pH 7.4) at a final DNA concentration of 100 nM. A total of 1 mL solution for each sample was transferred to a quartz cuvette. Dabcyl-labeled complementary strand or dummy strand (2 equiv.) in 2 μL PBS solution were added into the cuvette and rapidly mixed with a pipette. The fluorescence of the solution (ex=494 nm, em=522 nm) was continuously monitored every 3 seconds for 30 min. The endpoint was determined by adding a large excess (10 equiv.) of the complementary dabcyl strand to the mixture. The kinetics plots were normalized to the endpoint determined for each sample, and all measurements were repeated 3×.

Nuclease degradation kinetics. LNAs and pacLNAs were each mixed with their complementary dabcyl-labeled DNA (2 equiv.) in PBS. The solutions were heated to 95° C. for 5 min and cooled down to room temperature, then shaken overnight. Next, 100 μL of each sample was withdrawn and diluted to 1 mL (100 nM) with assay buffer (10 mM Tris-HCl, 2.5 mM MgCl₂, and 0.5 mM CaCl₂), pH 7.5). The mixture was transferred to a quartz cuvette which was mounted on a fluorimeter. DNase I was added and rapidly mixed to give a final concentration of 0.2 unit/mL. The fluorescence of the samples (ex=494 nm, em=522 nm) was measured immediately and every 3 seconds for 2 h. The endpoint was determined by adding a large excess of DNase I (5 units/mL) to the solution followed by incubation for 2 h.

Cell culture. NCI-H358 cells were cultured in RPMI 1640 media supplemented with 10% fetal bovine serum (FBS) and 1% antibiotics. All cells were cultured at 37° C. in a humidified atmosphere containing 5% CO₂.

Cellular uptake. Cellular uptake of LNAs and pacLNAs was evaluated using flow cytometry. Cells were seeded in 24-well plates at a density of 2.0×10⁵ cells per well in 1 mL full growth media and cultured for 24 h at 37° C. with 5% CO₂. After washing by PBS 1×, Cy3-labeled LNAs and pacLNAs (250 nM-5 μM equiv. of DNA) dissolved in serum-free culture media (400 μL) was added, and cells were further incubated at 37° C. for 4 h. Next, cells were washed with PBS 2× and treated with trypsin (60 μL per well). Thereafter, 1 mL of PBS was added to each culture well to suspend the cells. Cells were then analyzed on an Attune™ N×T flow cytometer (Invitrogen, MA). Data for 1.0×10⁴ gated events were collected.

Confocal Microscopy. Cells were seeded in 24-well glass bottom plates at a density of 1.0×10⁵ cells per well in 1 mL full growth media and cultured for 24 h at 37° C. with 5% CO₂. After washing by PBS 1×, Cy3-labeled LNAs and pacLNAs (2 μM equiv. of DNA) dissolved in serum-free culture media (400 μL) was added, and cells were further incubated at 37° C. for 4 h. Next, cells were washed with PBS 3× and fixed with 4% paraformaldehyde for 30 min at room temperature. After washed with PBS 3×, cells were stained with Hoechst 33342 for 10 min and imaged on an LSM-880 confocal laser scanning microscope (Carl Zeiss Ltd., Cambridge, UK). Imaging settings were kept identical for all samples in each study.

Western Blotting. Cells were seeded in 24-well plates at a density of 2.0×10⁵ cells per well in 1 mL full growth media and cultured for 24 h at 37° C. with 5% CO₂. After washing by PBS 1×, LNAs and pacLNAs (1 μM-10 μM equiv. of DNA) dissolved in full media (1 mL) was added, and cells were further incubated at 37° C. for 72 h. Next, cells were harvested and whole cell lysates were collected in 100 μL of RIPA cell lysis buffer supplemented with 1% phosphate inhibitor and 1% phophotase inhibitor. Total proteins in cell lysate were quantified using a bicinchoninic acid (BCA) protein assay kit. Equal amounts of total proteins (30 μg/lane) were separated on a 4-20% gradient SDS-PAGE gel and electro-transferred to nitrocellulose membrane. The membrane was then blocked with 3% bovine serum albumin (BSA) in Tris-buffered saline supplemented with 0.05% Tween-20 (TBST). After blocking, the membrane was cut according to the protein ruler and further incubated with appropriate primary antibodies overnight at 4° C. After washing with TBST 3×, the membrane was incubated with secondary antibodies at room temperature for 1 h. The detected proteins were visualized by chemiluminescence using the ECL Western Blotting Substrate (Bio-rad, MA, USA). Antibodies used in this study were: KRAS antibody (cat. NBP2-45536; Novus Biologicals), β-actin (cat. AM4302), anti-mouse IgG, HRP-linked antibody (cat. 7076S). Unless otherwise noted, antibodies were obtained from Cell Signaling Technologies.

MTT assay. The cell viability of NCI-H358 after treatment with LNAs, pacLNAs and bottlebrush polymer was analyzed by MTT (dimethylthiazol-diphenyltetrazolium bromide) colorimetric assay. Cells were seeded in 96-well plates at a density of 1×10⁴ cells per well in 175 μL full growth media and cultured for 24 h at 37° C. with 5% CO₂. Then cells were treated with LNAs, pacLNAs and bottlebrush polymer in the concentration range of 0.1-10 μM (equiv. of DNA). Cells treated with vehicle served as a control. After 48 h of incubation, 20 μL of 5 mg/mL MTT stock solution in PBS was added to each well. After incubation for another 4 h, the media was carefully removed. The resulting blue formazan crystals were dissolved in DMSO (200 μL per well), and measured at 490 nm on a BioTek® Synergy™ Neo2 Multi-Mode microplate reader (BioTek Inc., VT, USA).

Plasma pharmacokinetics (PK) studies. Animal protocols were approved by the Institutional Animal Care and Use Committee of Northeastern University. Animal experiments and operations were conducted in accordance with the approved guidelines. Immunocompetent C57BL/6 mice were used to examine the plasma PK of free LNAs (both PS and PO), pacLNAs, and free bottlebrush polymer lacking an ASO component. Mice were randomly divided into five groups (n=4). Cy5-labeled samples were intravenously (i.v.) administrated via the tail vein at equal ASO dosage (0.5 μmol/kg; free polymer concentration equals that of the pacDNAs). Of note, the fluorescence label is located on the ASO component except for the free polymer. Blood samples (25 μL) were collected from the submandibular vein at varying time points (30 min, 2 h, 4 h, 10 h, 24 h, 48 h and 72 h) using BD Vacutainer™□ blood collection tubes with lithium heparin. Heparinized plasma was obtained by centrifugation at 3000 rpm for 20 min, aliquoted into a 96-well plate, and measured for fluorescence intensity on a Synergy™ Neo2 Multi-Mode microplate reader (BioTek Instruments Inc., VT, USA). The amounts of ASO in the blood samples were estimated using standard curves established for each sample. To establish the standard curves, samples of known quantities were incubated with freshly collected plasma for 1 h at room temperature before fluorescence was measured.

NCI-H358 xenograft tumor model. To establish the NCI-H358 xenograft tumor model, approximately 5×10⁶ cells in 100 μL phosphate buffered saline (PBS) were implanted subcutaneously on the right flank of 6-week-old athymic mice. Mice were monitored for tumor growth every other day.

Whole-animal and ex vivo organ imaging. NCI-H358 xenograft-bearing athymic mice were i.v. injected with Cy5-labeled samples at an ASO dose of 0.5 μmol/kg. Then mice were scanned at 1, 4, 8, 24 h, and daily thereafter until 13 weeks or until fluorescence is no longer observable using an IVIS Lumina II imaging system (Caliper Life Sciences, Inc. MA, USA). To evaluate the biodistribution of pacDNAs and the bottlebrush polymer, mice were euthanized using CO₂, and major organs and the tumor were dissected for biodistribution analysis. For the analysis of tumor penetration depth, tumors were immediately frozen in O.C.T compound (Fisher Scientific Inc., USA) 24 h after injection. The frozen tumor tissues were cut into 8 μm-thick sections, stained with Hoechst 33342 and imaged on an LSM-880 confocal laser scanning microscope (Carl Zeiss Ltd., Cambridge, UK).

Antitumor efficacy in NCI-H358 xenograft-bearing mice. To screen the pacLNAs in antitumor efficacy, an NCI-H358 subcutaneous xenograft model was first established. When the tumor volume reached ca. 100 mm³, mice were randomly divided into four groups (n=5) and treated with vehicle (PBS), PO pacLNA, PS pacLNA and scramble PO pacLNA via the tail vein at the concentration of 0.5 μmol/kg. Samples were injected once a week until day 36. The volume of tumors and weight of mice were recorded every 3 days and 3 more times after the last treatment. Antitumor activity was evaluated in terms of tumor size by measuring two orthogonal diameters at various time points (V=0.5×ab²; a: long diameter, b, short diameter). At day 36, mice were euthanized with CO₂, and tumors and major organs (heart, lung, liver, spleen, and kidney) from each group were excised, fixed in 4% paraformaldehyde/PBS for 6 h, and placed into a 30% sucrose/PBS solution overnight at 4° C. The fixed tissues were paraffin-embedded and cut into 8 μm-thick sections with a cryostat. The sections were then processed with hematoxylin and eosin (H&E) staining. Immunohistochemistry staining of KRAS was carried out using mouse anti-KRAS primary antibody (1:1000 dilution, Invitrogen Co., CA, USA) and goat anti-mouse secondary antibody (1:5000 dilution, ThermoFisher, MA, USA).

### Example 1. Physicochemical Properties of pacDNA

In an embodiment of the disclosure, the ASO sequence of choice is the same as that of AZD4785, a cEt-modified clinical compound targeting the 3′ untranslated region (3′ UTR) of the KRAS mRNA (FIG. 1A). Although the targeted region is away from mutation sites (thus wild-type KRAS is also depleted), AZD4785 has shown selectivity toward KRAS^{MUT} cells for inhibiting proliferation and is potent against several mutant isoforms. However, a Phase I clinical study of AZD4785 was unsuccessful due to insufficient target depletion. Indeed, the oncological use of ASOs is challenged by their short plasma half-life and limited tumor site accumulation, necessitating frequent injections which can cause undesirable peak-to-valley fluctuations of drug concentration, increased non-antisense side effects, reduced patient compliance, and increased cost. Adopting the same sequence as AZD4785 for pacDNA allows for direct comparisons with an existing body of preclinical data.

A library of PEGylated ASO structures was designed to elucidate the in vivo importance of various structural parameters and to optimize ASO potency and pharmacological properties. These pacDNA structures vary in ASO composition (natural and chemically modified), conjugation site (sequence termini or internal position), and releasability (stable or bioreductively cleavable) (see, e.g., FIGS. 1B, 1C, and 1D; Table 1). Additionally, a Y-shaped PEG (40 kDa), which has been adopted in the oligonucleotide drug, pegaptanib (brand name Macugen®) is used to form an ASO conjugate as a polymer architecture control.

TABLE 1
Oligonucleotide strands used in this study.

| Strand description | Sequence |
|---|---|
| DBCO-modified AS | 5′-DBCO-GCTATTAGGAGTCTTT-3′ |
| | (SEQ ID NO: 1) |
| Fluorescein-labeled | 5′-DBCO-GCTATTAGGAGTCTTT-FL-3′ |
| DBCO-modified AS | (SEQ ID NO: 2) |
| Cy3-labeled and DBCO- | 5′-DBCO-GCTATTAGGAGTCTTT-Cy3-3′ |
| modified AS | (SEQ ID NO: 3) |
| Cy5-labeled and DBCO- | 5′-DBCO-GCTATTAGGAGTCTTT-Cy5-3′ |
| modified AS | (SEQ ID NO: 4) |
| Dabcyl-labeled sense | 5′-Dabcyl-AAAGACTCCTAATAGC-3′ |
| | (SEQ ID NO: 5) |
| Dabcyl-labeled | 5′-Dabcyl-ACGACTAGTATCACAA-3′ |
| scrambled sense | (SEQ ID NO: 6) |
| Amine-modified PS AS | 5′-NH₂-G*C*T*A*T*T*A*G*G*A*G*T*C*T*T*T-3′ |
| | (SEQ ID NO: 7) |
| DBCO-modified PS AS | 5′-DBCO-G*C*T*A*T*T*A*G*G*A*G*T*C*T*T*T-3′ |
| | (SEQ ID NO: 8) |
| Fluorescein-labeled | 5′-DBCO-G*C*T*A*T*T*A*G*G*A*G*T*C*T*T*T*-FL-3′ |
| DBCO-modified PS AS | (SEQ ID NO: 9) |
| Cy3-labeled and DBCO- | 5′-DBCO-G*C*T*A*T*T*A*G*G*A*G*T*C*T*T*T*-Cy3-3′ |
| modified PS AS | (SEQ ID NO: 10) |
| Cy5-labeled and DBCO- | 5′-DBCO-G*C*T*A*T*T*A*G*G*A*G*T*C*T*T*T*-Cy5-3′ |
| modified PS AS | (SEQ ID NO: 11) |
| Fluorescein-labeled | 5′-NH₂-G*C*T*A*T*T*A*G*G*A*G*T*C*T*T*T*-FL-3′ |
| amine-modified PS AS | (SEQ ID NO: 12) |
| Cy3-labeled and amine- | 5′-NH₂-G*C*T*A*T*T*A*G*G*A*G*T*C*T*T*T*-Cy3-3′ |
| modified PS AS | (SEQ ID NO: 13) |
| Cy5-labeled and amine- | 5′-NH₂-G*C*T*A*T*T*A*G*G*A*G*T*C*T*T*T*-Cy5-3′ |
| modified PS AS | (SEQ ID NO: 14) |
| PS AS containing | 5′-G*C*T*A*T*T*A*G*G*A*G*T*C*T*T*T-3′ |
| amino-modifier (N₂- | (SEQ ID NO: 15) |
| amine modification-C6-dG) | |
| Fluorescein-labeled PS | 5′-G*C*T*A*T*T*A*G*G*A*G*T*C*T*T*T*-FL-3′ |
| AS containing amino- | (SEQ ID NO: 16) |
| modifier (N₂-amine | |
| modification-C6-dG) | |
| Cy3-labeled PS AS | 5′-G*C*T*A*T*T*A*G*G*A*G*T*C*T*T*T*-Cy3-3′ |
| containing amino- | (SEQ ID NO: 17) |
| modifier (N₂-amine | |
| modification-C6-dG) | |
| Cy5-labeled PS AS | 5′-G*C*T*A*T*T*A*G*G*A*G*T*C*T*T*T*-Cy5-3′ |
| containing amino- | (SEQ ID NO: 18) |
| modifier (N₂-amine | |
| modification-C6-dG) | |
| Scrambled PS | 5′-A*T*G*T*C*C*G*T*T*G*T*G*T*A*T*A-3′ |
| | (SEQ ID NO: 19) |
| Dabcyl-labeled sense | 5′-Dabcyl-A*A*A*G*A*C*T*C*C*T*A*A*T*A*G*C-3′ |
| | (SEQ ID NO: 20) |

AS: antisense; FL: fluorescein; asterisk (*): phosphorothioate internucleotide linkage; underline: N2 amine-modified nucleobase for polymer conjugation

The brush polymer was prepared via sequential ring-opening metathesis polymerization (ROMP) of 7-oxanorbornenyl bromide (ON—Br) and norbornenyl PEG (N-PEG), to yield a diblock architecture (pONBr₅-b-pNPEG₃₀, polydispersity index <1.2). Following azide substitution and subsequent coupling with dibenzocyclooctyne (DBCO)-modified ASO strands via the strain-promoted copper-free click chemistry, pacDNAs structures with an average of 2.0 ASO strands per polymer were prepared (˜95% yield, FIGS. 1E, 1F, and 1G).

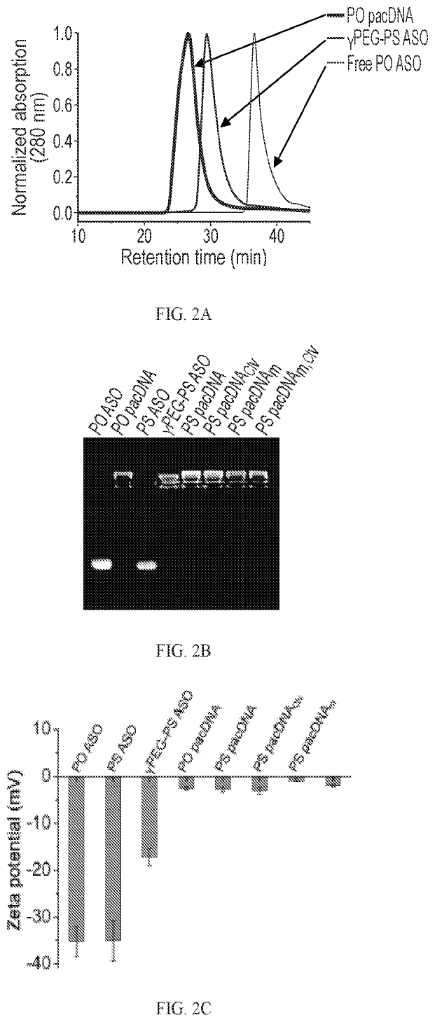

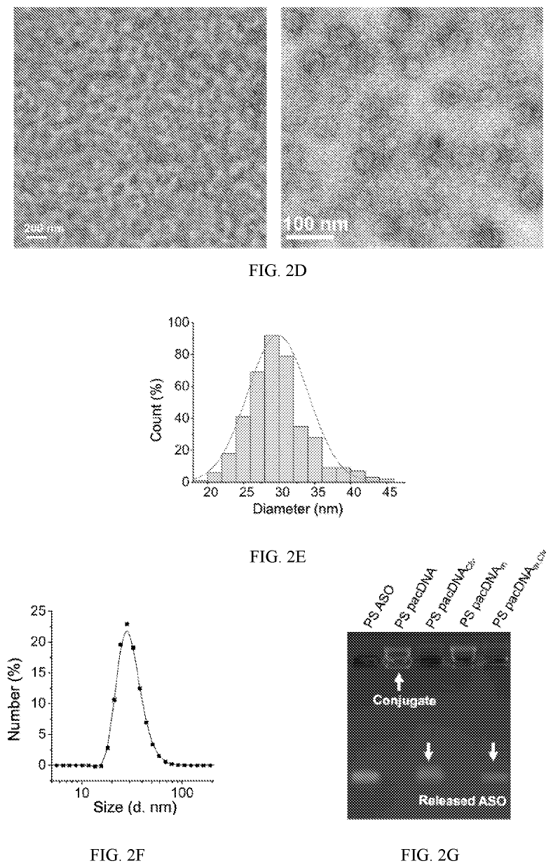

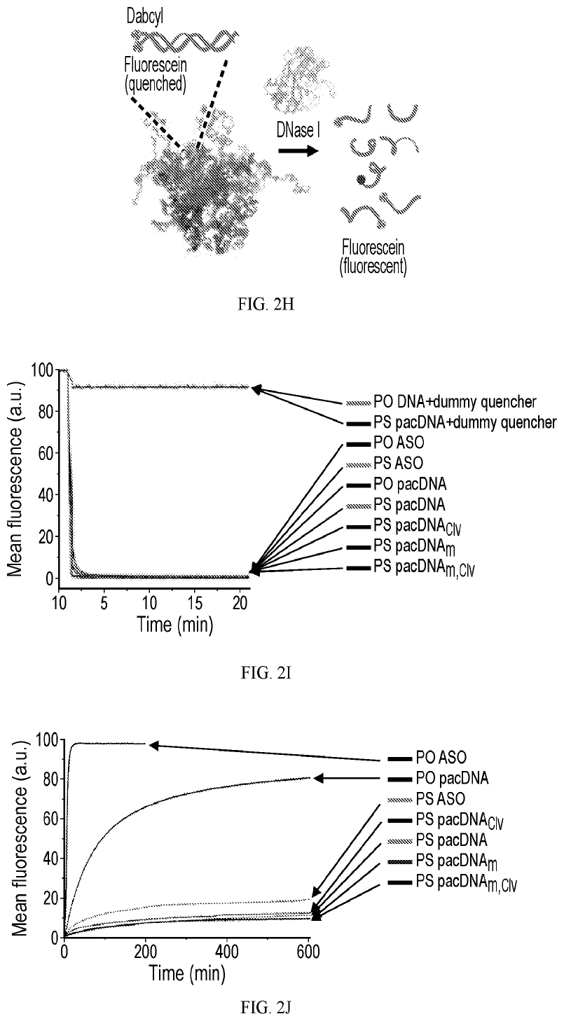

The conjugates were purified by aqueous size exclusion chromatography (SEC, FIG. 2A) and lyophilized for storage. Agarose gel electrophoresis (AGE, 1%) indicates the successful synthesis of the pacDNA and the Y-shaped PEG-ASO conjugates, which are free of unconjugated ASO (FIG. 2B). The upward gel migration of the pacDNA is a consequence of the transient interaction of PEG with cations in the buffer, and not because of a net positive charge. Indeed, ζ potential measurements indicate that the pacDNAs have a slight negative charge (−1 to −3 mV) in Nanopure™ water, which is significantly below that of free DNA (˜−35 mV) and the Y-shaped PEG-ASO conjugate (˜−17 mV, FIG. 2C). Being molecular nanoparticles, the pacDNAs exhibit a spherical morphology with a dry-state diameter of ˜29 nm, as evidenced by transmission electron microscopy (TEM) (FIGS. 2D and 2E). The size is consistent with dynamic light scattering (DLS) measurements, which show a number-average hydrodynamic diameter of ˜30 nm in Nanopure™ water (FIG. 2F). The redox-responsiveness of pacDNAs with cleavable linkages was tested by treatment with 10 mM dithiol threitol (DTT) in phosphate-buffered saline (PBS) at 37° C., a condition often adopted to mimic the reducing intracellular environment (FIG. 2G). AGE shows that ˜80% of the DNA is released after 30 min of treatment, as determined by gel densitometry analysis. In contrast, the same treatment for non-cleavable pacDNAs resulted in no release of the DNA.

A hallmark feature of the pacDNA is its ability to hybridize with the complementary target in kinetically and thermodynamically the same manner as free DNA, but is able to resist protein binding. This feature was verified using a fluorescence quenching assay, in which a quencher (dabcyl)-modified sense strand is added to fluorescein-labeled antisense pacDNA. Upon hybridization, the fluorescence is quenched due to the spatial proximity of the fluorophore-quencher pair, and the rate of which is indicative of the hybridization kinetics (FIG. 2H). All pacDNA conjugates, the Y-shaped conjugate, and free ASO hybridize with the sense strand rapidly with a negligible difference (FIG. 2I). When a scrambled dabcyl-DNA sequence was added, fluorescent signals were not affected, ruling out nonspecific binding. To investigate the extent of protein access, DNase I (an endonuclease mainly for dsDNA) was added to prehybridized fluorophore/quencher-bearing duplexes. With DNase I action, an increase of fluorescence is expected, reflecting the nucleolytic degradation rate. As shown in FIG. 2J, the phosphodiester (PO) pacDNA exhibits a significantly extended half-life (t_{1/2}) of ˜92 min compared with free PO DNA, which is degraded rapidly with a t_{1/2} of ˜ 5 min. On the other hand, both the PS pacDNA and the naked PS ASO exhibit very limited enzymatic degradation, with 11.5% and 19.3% degraded after 10 hours of treatment, respectively, which is in line with the typical nuclease resistance of PS oligonucleotides.

### Example 2. Cellular Uptake, KRAS Depletion, and Cell Viability

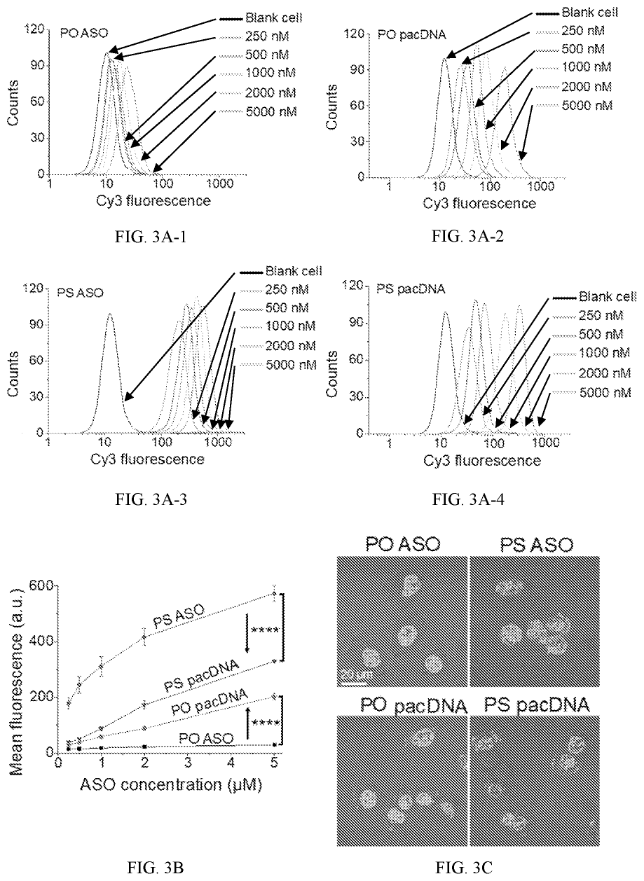

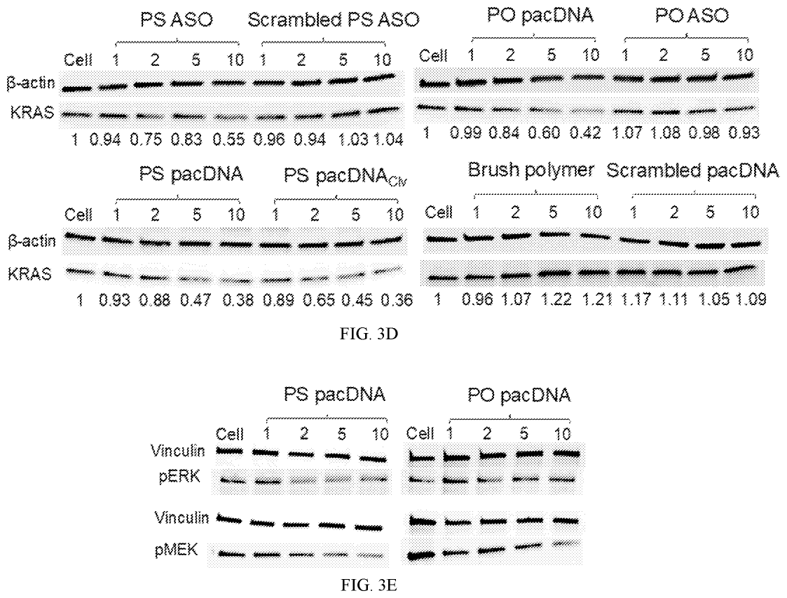

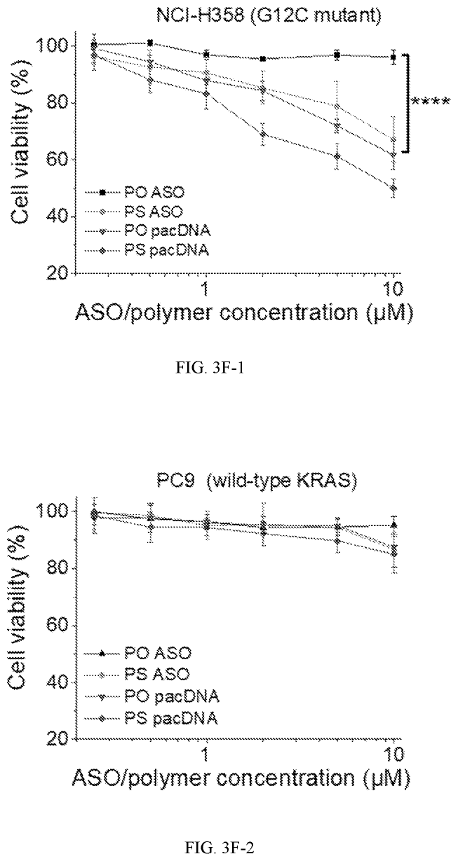

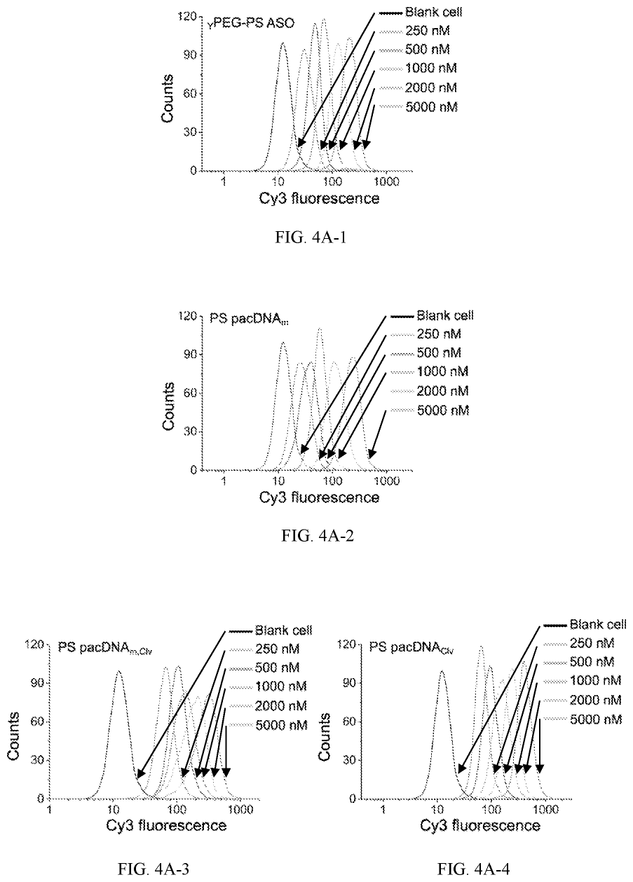

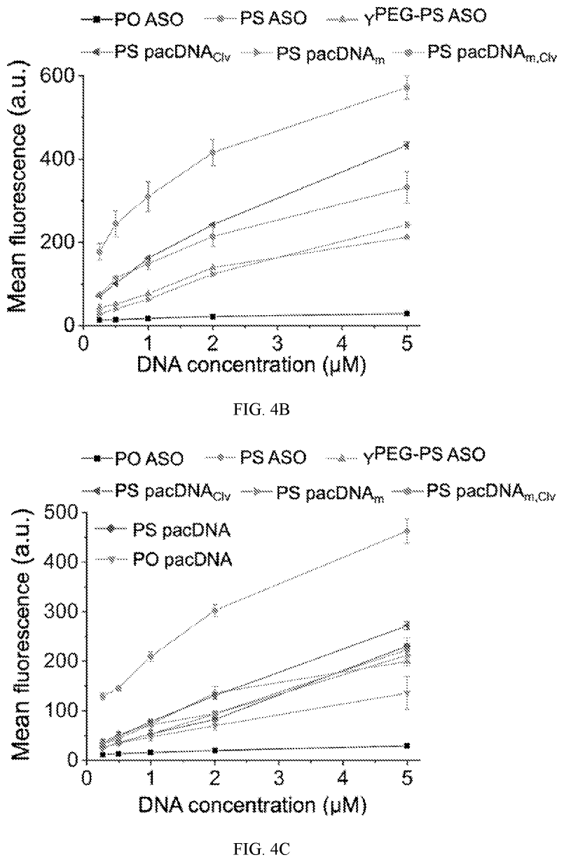

One of the most significant restraints to the use of ASOs for pharmacological purposes is their limited cellular uptake and localization in the appropriate intracellular compartments. To investigate the cellular uptake of pacDNA, NCI-H358 cells (a KRAS^{G12C} NSCLC line) were treated with cyanine 3 (Cy3)-labeled pacDNA or free ASO for 4 h in serum-free media. Oligonucleotides with natural PO internucleotide linkages typically do not traverse the lipophilic cell membrane passively due to their highly polyanionic nature. On the other hand, PS ASOs bind promiscuously to proteins (e.g., membrane and serum proteins), which ultimately results in high endocytosis but also increased the potential for off-target effects in vivo. Indeed, naked PS ASO exhibits ˜30× higher uptake rate by NCI-H358 cells compared to the PO ASO (FIGS. 3A-1, 3A-2, 3A-3, 3A-4, and 3B). However, the PS pacDNA is internalized by the cells only ˜1.6× faster than PO pacDNA, and the latter is taken up ˜10× faster than the naked PO ASO (FIGS. 3A-1, 3A-2, 3A-3, 3A-4, and 3B). It was hypothesized that the bottlebrush polymer produces a “leveling” effect, which increases PO ASO uptake while reducing that of the PS ASO (both towards the intrinsic uptake level of the unmodified polymer). These results suggest that the brush polymer reduces the dependency on ASO chemistry for cellular uptake and can generate a more predictable uptake pattern irrespective of the ASO (FIGS. 3A-1, 3A-2, 3A-3, 3A-4, and 3B, and FIGS. 4A and 4B). Confocal microscopy confirmed that in all cases the pacDNAs are internalized by the cell as opposed to being surface bound (FIG. 3C), although the punctate appearance of fluorescence signals suggests predominant distribution in endosomal structures. The presence of serum in cell culture media did not change these trends in cell uptake (FIG. 4C). To examine the uptake pathway, pharmaceutical endocytosis inhibitors were used to block the established pathways and their contribution was assessed using flow cytometry (Table 2). The results indicate energy-dependent, mixed uptake mechanisms that likely involve macropinocytosis and clathrin-mediated endocytosis (FIGS. 5A-1, 5A-2, and 5A-3).

TABLE 2
Pharmacological uptake inhibitors used in this study.

| Target | Chemical blocker | Conc. | |
|---|---|---|---|
| Clathrin | Chlorpromazine (CPM) | 1, 5 | μg/mL |
| Lipid raft/caveolae | Methyl-β-cyclodextrin (MβCD) | 5, 10 | mg/mL |
| Micropinocytosis | Rottlerin | 1, 3 | μg/mL |
| ATP | Sodium azide (NaN₃) | 10, 50 | mM |

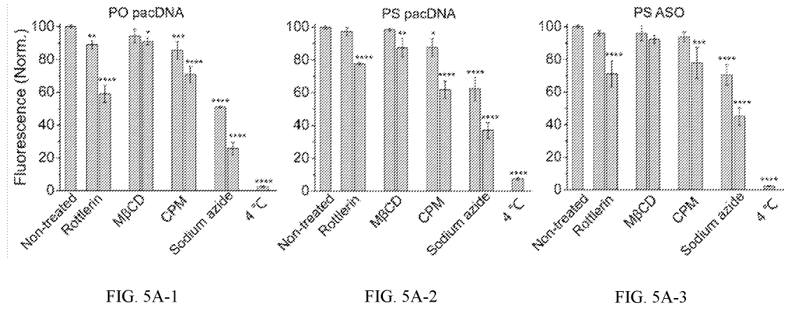

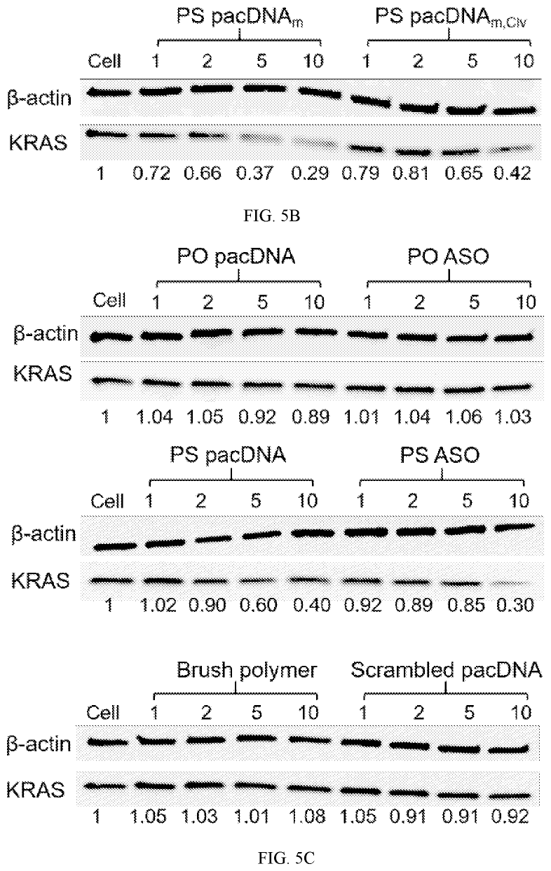

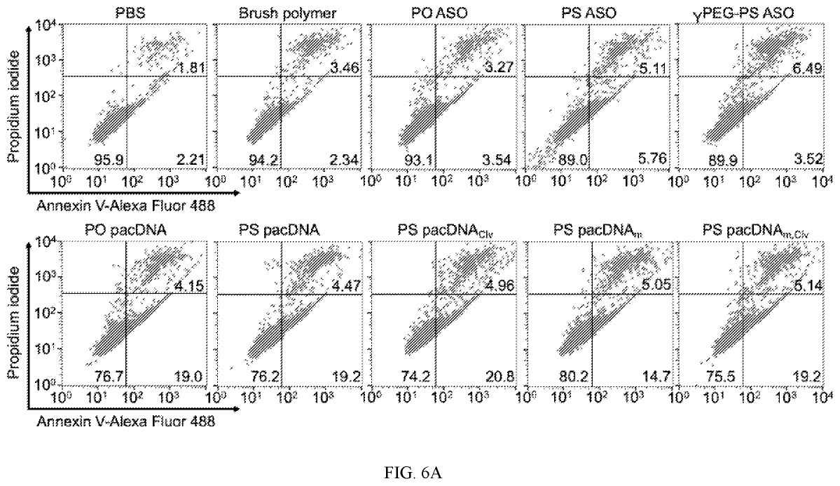

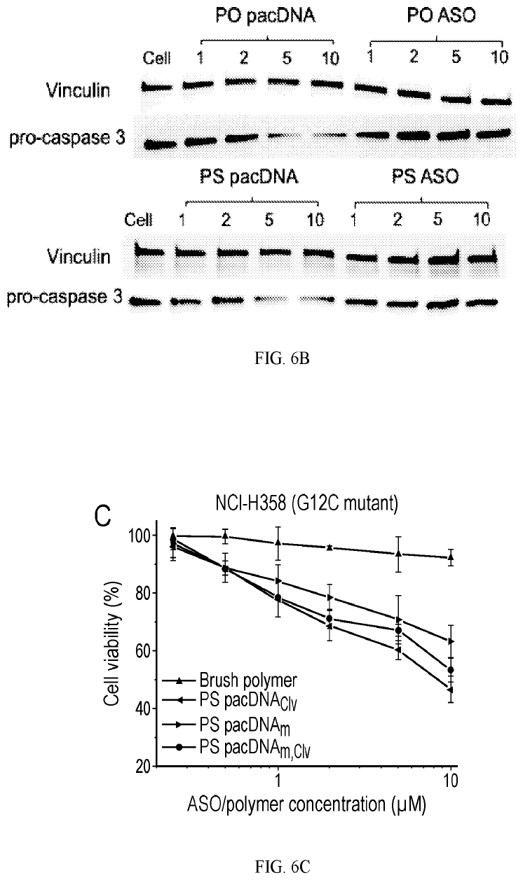

To study the antisense activity of pacDNA and associated phenotypic response, two cell lines, NCI-H358 (KRAS^{G12C}) and PC9 (wild-type), were treated with pacDNAs and controls at concentrations ranging from 1 to 10 μM (ASO basis). Western blot of cell lysates shows dose-dependent downregulation of KRAS for all pacDNA structures containing a correct ASO sequence, while a scrambled PS pacDNA and the bottlebrush polymer alone do not lead to apparent downregulation (FIGS. 3D and 5B). Target depletion is generally >50% irrespective of ASO chemistry, conjugation site, or releasability when the pacDNA concentration is greater than 5 μM. Notably, the pacDNAs exhibited stronger target depletion than the naked PS ASO, despite the latter showing the highest level of cellular uptake. While the pacDNAs were able to knock down KRAS in both cell lines, only NCI-H358 cells have shown significant dependency on KRAS for viability; the growth of PC9 cells is nearly unaffected by the treatment (FIGS. 3F, 5C, and 6C), which is consistent with previous studies. Among the pacDNA structures, the PS pacDNAs (PS pacDNA, PS pacDNA_{Clv}, and PS pacDNAₘ) appear to be marginally more effective than the PO counterpart (PO pacDNA). The downregulation of KRAS in NCI-H358 cells was followed by inhibition of downstream mitogen-activated protein kinase (MAPK) pathway signaling including downregulation of phosphor-MAPK kinase (pMEK) and phosphor-extracellular signal-regulated kinase (pERK) (FIG. 3E), and increased apoptosis (FIG. 6A). FITC-annexin V/propidium iodide (PI) staining of cells treated with pacDNAs shows increased induction of apoptosis for all pacDNA variations (>22%), with the majority of the apoptotic cells in the early phase, while treatment with free PO DNA and the bottlebrush polymer does not result in appreciable changes relative to untreated cells. In addition, induction of pro-caspase-3 cleavage upon KRAS depletion was observed in a dose-dependent manner for NCI-H358 cells (FIG. 6B). Collectively, these data suggest that pacDNA downregulates both mutant and wild-type KRAS isoforms and elicits selective phenotypic responses in KRAS^{MUT} cells.

### Example 3. Plasma PK, Biodistribution, Antitumor Efficacy, and Safety

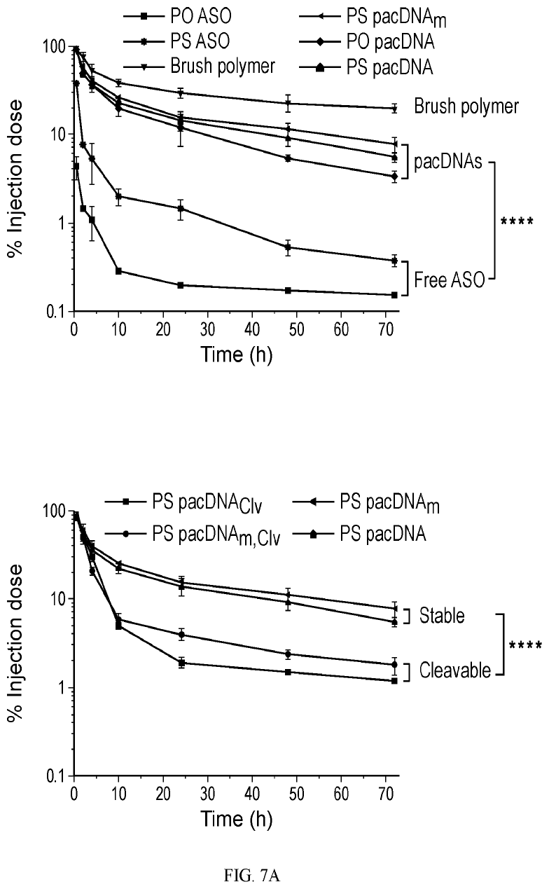

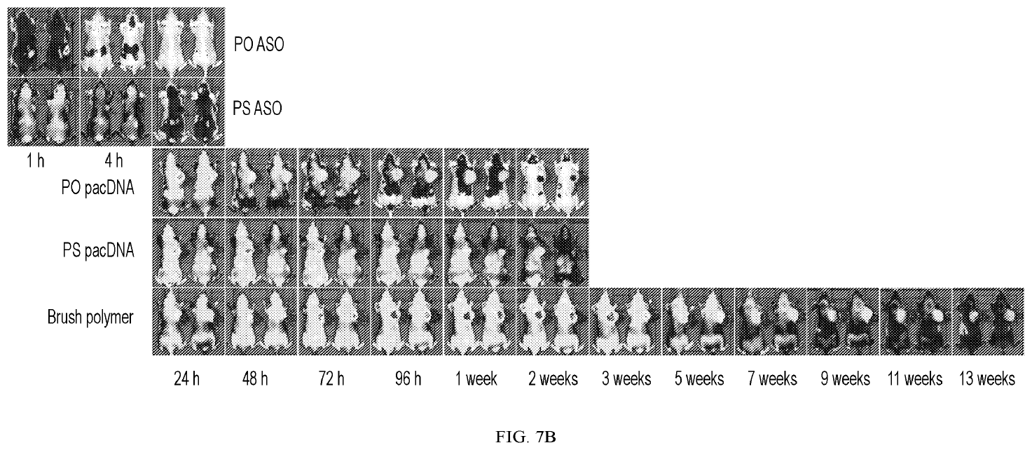

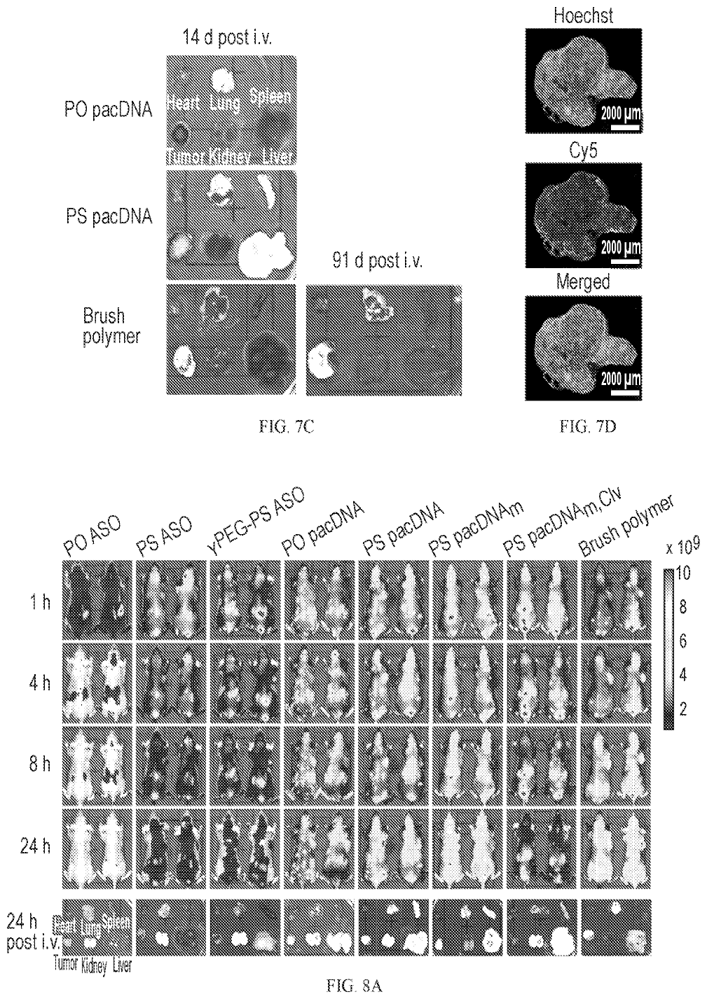

To assess the plasma PK of the pacDNA, blood samples from C57BL/6 mice dosed intravenously (i.v.) with Cy5-labeled pacDNA and controls were collected and analyzed for up to 72 h. Free ASOs are cleared rapidly via renal glomerular filtration with very short elimination half-lives (t_{1/2β,PO}=0.86 h, t_{1/2β,PS}=1.2 h; two-compartment model, FIG. 7A and Table 3). In sharp contrast, all samples containing the bottlebrush polymers show markedly longer t_{1/2β}(˜14-23 h), among which the free polymer exhibits the greatest level of blood retention, with ˜20% of the injected dose remaining in circulation at 72 h (FIG. 7A). Among the pacDNAs, three observations are made: 1) the stable, non-cleavable pacDNAs (PO pacDNA, PS pacDNA, and PS pacDNAₘ) show better plasma retention than the bioreductively cleavable counterparts (PS pacDNA_{Clv} and PS pacDNA_{m,Clv}); 2) the PS pacDNA is retained more than the PO pacDNA; 3) mid-chain anchored pacDNA (PS pacDNAₘ) circulates longer than the terminus-anchored version (PS pacDNA). These results suggest that the steric shielding by the bottlebrush polymer is not absolute; the enzymatic and chemical stability of the ASO as well as the level of its exposure to plasma components remain secondary contributing factors for PK. Nonetheless, the bottlebrush polymer decidedly elevates ASO blood concentration and bioavailability compared to naked ASOs with an improvement of 1-2 orders of magnitude if measured by the area under the curve (AUC_{∞}).

TABLE 3
Plasma pharmacokinetic parameters in C57BL/6 mice.

| Sample | t_{1/2} (α) (h) | t_{1/2} (β) (h) | AUC_{∞} (nmol/ml · h) |
|---|---|---|---|
| PO ASO | 0.21 | 0.86 | 1.4 |
| PS ASO | 0.40 | 1.2 | 7.8 |
| _{γ}PEG-PS ASO | 0.93 | 1.7 | 16.9 |
| PO pacDNA | 0.93 | 14.0 | 53.9 |
| PS pacDNA | 0.85 | 15.5 | 65.0 |
| PS pacDNA_{Clv} | 1.6 | 4.5 | 24.4 |
| PS pacDNAₘ | 1.3 | 15.4 | 75.6 |
| PS pacDNA_{m, Clv} | 1.4 | 4.6 | 27.5 |
| Bottlebrush polymer | 1.9 | 22.7 | 127.6 |

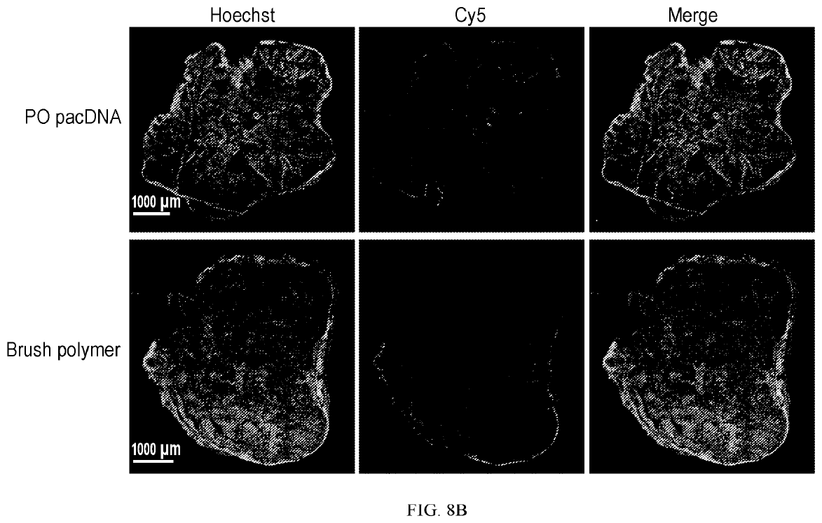

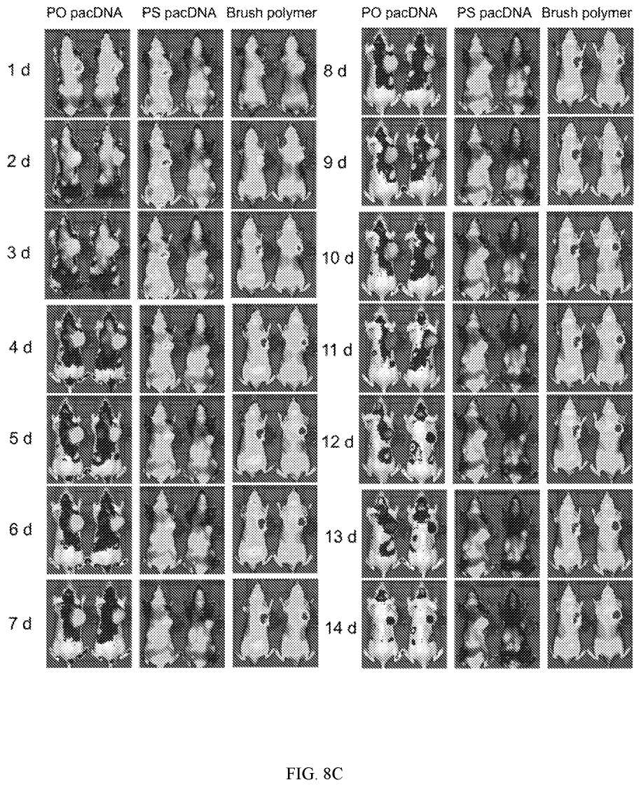

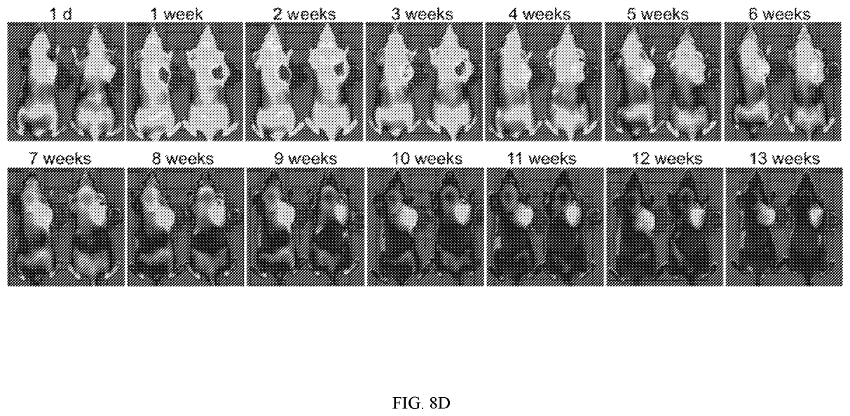

One outcome of the elevated plasma PK is access to passive targeting of highly vascularized tissues such as certain tumors, likely via the enhanced permeation and retention (EPR) effect. To assess the biodistribution of pacDNA and controls, BALB/C-nu/nu mice bearing subcutaneous NCI-H358 xenografts were injected i.v. with Cy5-labeled pacDNAs and controls. Fluorescence imaging of both live animals and the dissected organs 24 h post-injection confirms that free PO ASO is quickly and primarily cleared by the kidney, while the PS ASO is cleared by both the kidney and the liver, with weak signals at the tumor site (FIGS. 7B and 8A). The Y-shaped PEG-PS ASO conjugate does not cause apparent changes in biodistribution relative to the parent ASO. Conversely, in stable pacDNA- and brush polymer-treated mice, strong fluorescence signals are apparent throughout the entire animal body at 24 h, and tumor site accumulation is evident. Confocal microscopy of cryosectioned tumor slices reveals significant ASO signals not only on the periphery of the tumor but also within the depths of the solid tumor (FIGS. 7D and 8B). It is found that the bioreductively cleavable conjugate (pacDNA_{m,Clv}) shows faster clearance and less tumor accumulation compared to the stable pacDNAs, possibly due to inadvertent release while in blood circulation, leading to liver/renal clearance (FIG. 8A). The clearance of the samples from mice was monitored after a single i.v. injection by imaging the animals daily (FIGS. 8C and 8D). Astonishingly, the free bottlebrush polymer reached peak signal intensities 4 days after injection (FIGS. 7B and 8C), and the peak level persisted for 3 weeks before slowly declining (FIG. 8D). The tissue with the slowest clearance rate is the tumor, in which the bottlebrush polymer persisted for at least 13 weeks (FIGS. 7B and 8D). In comparison, both PO and PS pacDNA were cleared in 1-2 weeks (still significantly enhanced relative to their parent ASOs, FIGS. 7B and 8C). The PO pacDNA showed more pronounced tumor-associated fluorescence than the PS version, possibly because the PS ASO, even when shielded by the bottlebrush polymer, still retains a propensity for non-specific binding with proteins, leading to recognition and uptake by the mononuclear phagocyte system. Indeed, fluorescence imaging of the dissected organs two weeks post-injection shows that the PO pacDNA accumulates predominantly in the tumor, liver, and kidney, whereas the PS pacDNA exists in the highest abundance in the spleen and liver, followed by the tumor (FIG. 7C). Collectively, these data indicate that the bottlebrush polymer is a long-circulating, long-retention vector which can partially impart these properties to conjugated ASOs, making them viable for systemic delivery.

### Example 4. Antitumor Efficacy of pacDNA

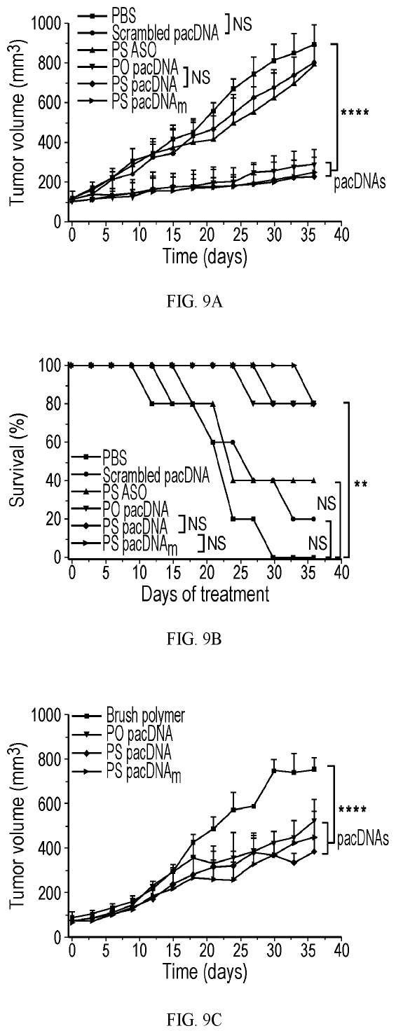

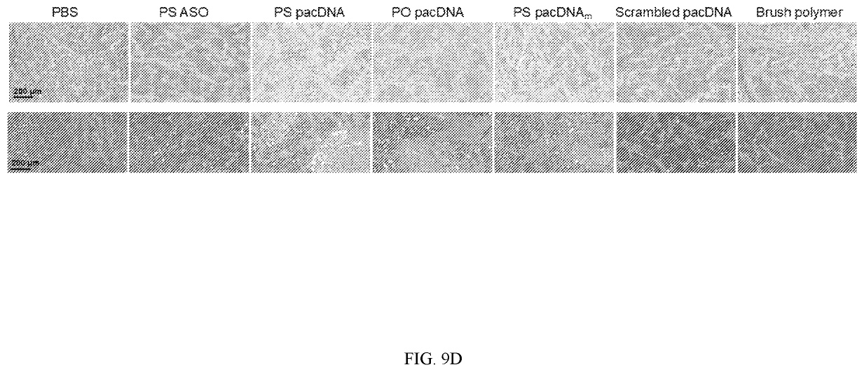

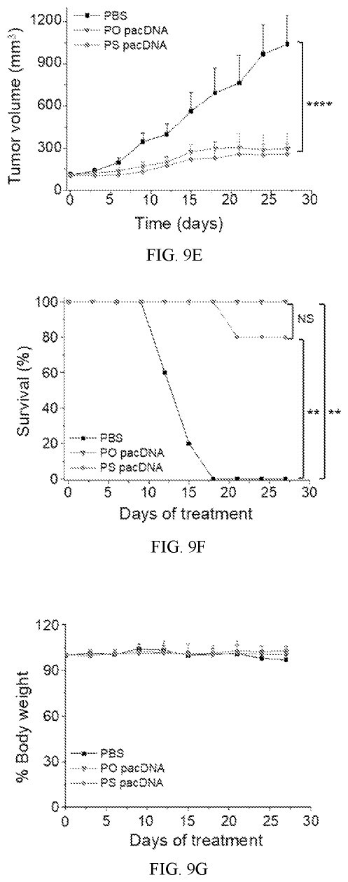

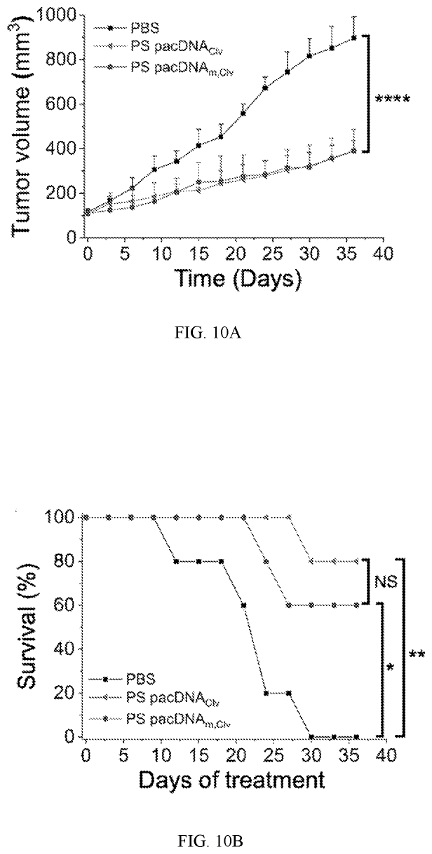

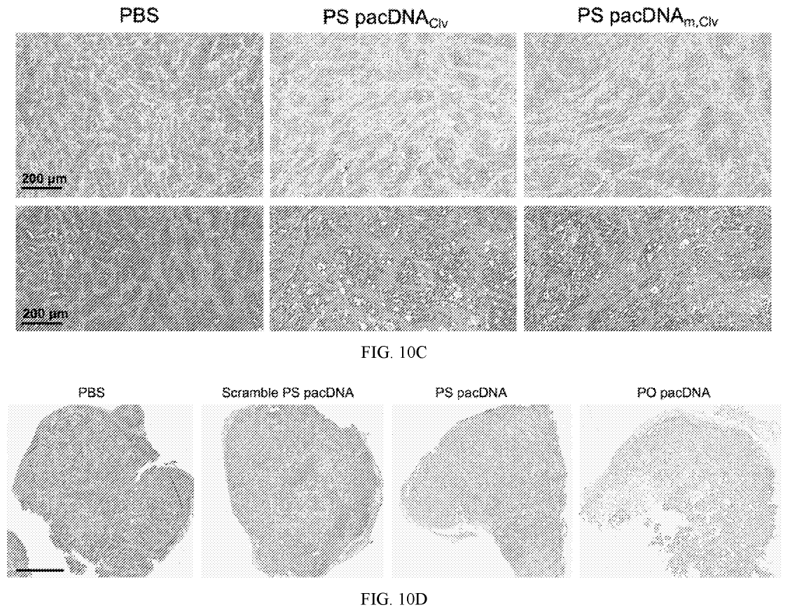

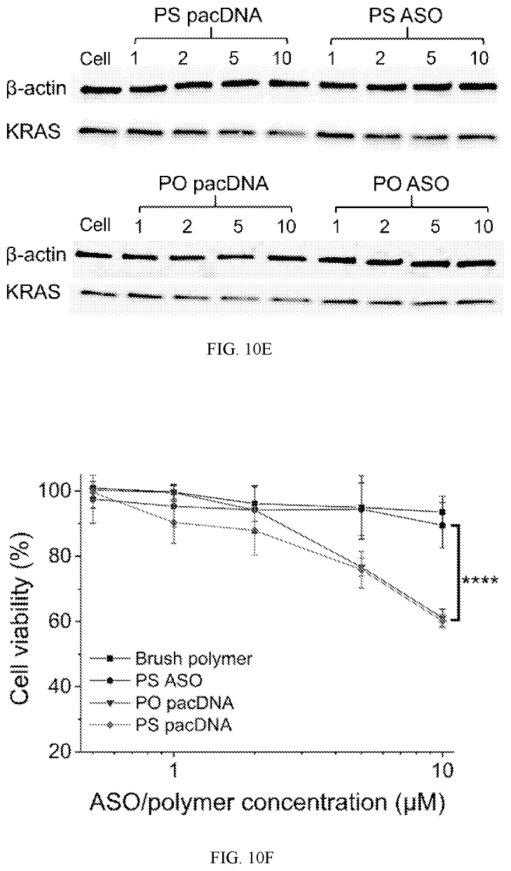

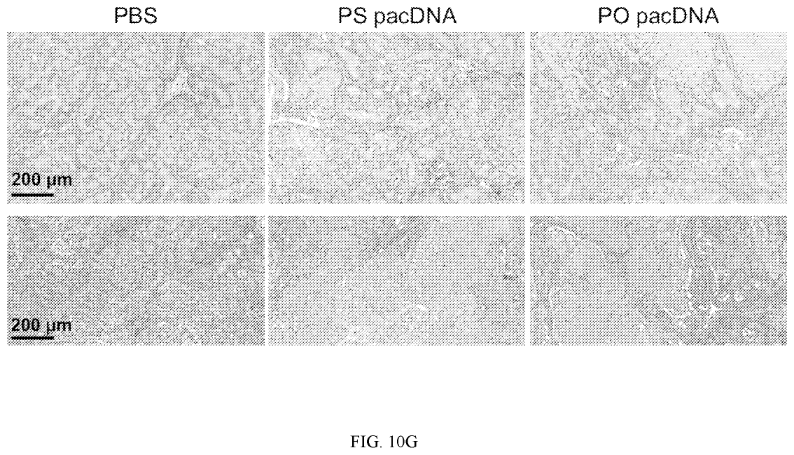

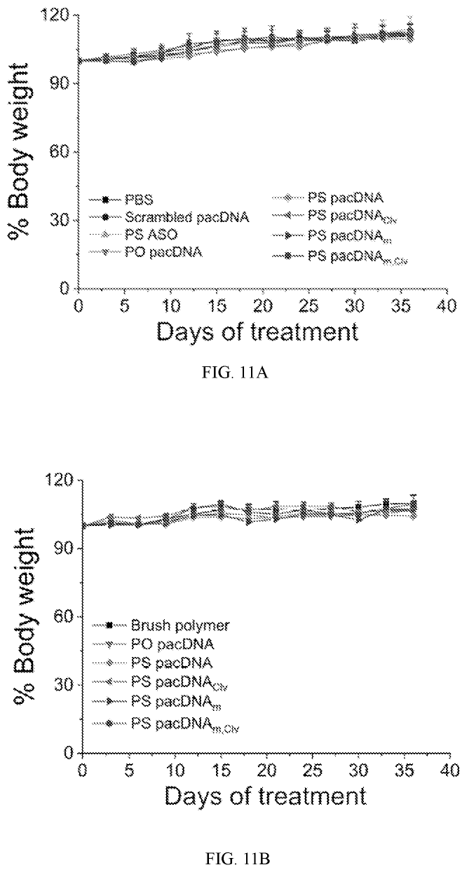

The antitumor efficacy of the pacDNA was assessed in male BALB/c nu/nu mice bearing subcutaneous NCI-H358 xenografts. When the xenografts reached a volume of ca. 100 mm³, pacDNAs, free ASOs, or vehicle (PBS) were administered i.v. (0.5 μmol/kg) once every 3^{rd} day for a total of 12 doses. By day 36, the average tumor volume in the vehicle-treated groups has progressed to ˜900 mm³. Remarkably, all pacDNA structures triggered potent tumor growth inhibition (averaging 230-390 mm³, FIGS. 9A and 10A), irrespective of ASO conjugation site, chemical modification, and releasability. While the cleavable pacDNAs (PS pacDNA_{Clv} and PS pacDNA_{m,Clv}) appear to be slightly less potent than the stable forms (PO pacDNA, PS pacDNA, and PS pacDNAₘ), statistical analysis shows that the difference in tumor size among the pacDNA-treated groups to be insignificant. To rule out non-specific antitumor activity from either the PS modification or the polymer component (or the enhanced tumor site delivery of PS oligonucleotides by the polymer), free PS ASO and a PS pacDNA with a scrambled sequence were used as negative controls; both resulted in insignificant antitumor response (FIG. 9A). Kaplan-Meier survival analysis (using an increase in tumor size of fourfold as a surrogate for survival endpoint, FIGS. 9B and 10B) shows that treatment with pacDNAs delays the time to reach the surrogate endpoint compared to the control groups. Immunohistostaining reveals that pacDNAs induced a marked reduction in KRAS protein levels in the tumor tissues after the last treatment (FIGS. 9D, 10C, and 10D). Consistently, histological analyses by hematoxylin and eosin (H&E) staining demonstrated severe loss of tumor cellularity in cases treated with KRAS-targeting pacDNAs compared to the cases from the control groups (FIGS. 9D and 10C). These data strongly corroborate earlier in vitro indication that the pacDNA is able to relax the requirement on ASO chemistry, allowing natural, PO ASO to attain comparable efficacy as chemically modified ASOs. The results are particularly significant when one compares them with the preclinical evaluation of AZD4785 in an identical tumor model. The clinical ASO with cEt modification was able to reduce tumor growth to a very similar extent as the pacDNA. However, the overall dosage of the pacDNA throughout the treatment period is only 0.025× that of AZD4785, which was dosed at 10 μmol/kg with a schedule of 5 subcutaneous injections per week.

To further explore the minimal effective dosage, a reduced-dosage study was performed in which the pacDNAs were administered at 0.1 μmol/kg once every 3^{rd} day for a total of 12 i.v. injections. At 0.005× the dosage of AZD4785, the pacDNAs (PO pacDNA, PS pacDNA, and PS pacDNAₘ) are still able to produce a statistically significant phenotypic response, although a dose-dependency in tumor size is evident (FIG. 9C). Notably, inhibition was not apparent until ˜17 days into the treatment, which is possibly due to the accumulation of the pacDNA at the tumor site allowing for a critical concentration to be reached after several dosages. Overall, a massive increase in ASO bioactivity associated with the pacDNA to the improved PK and reduced non-antisense binding with proteins and cells was observed.

To demonstrate the antitumor activities of the pacDNA against different mutant KRAS isoforms, a subcutaneous NCI-H1944 xenograft model, which carries the KRAS^{G13D} mutation, was established. In vitro studies with both the PS and the PO pacDNAs confirm KRAS downregulation and proliferation inhibition against NCI-H1944 cells, while the free PS ASO and the bottlebrush polymer show negligible inhibition (FIG. 10E). Systemic delivery of the pacDNAs (PO and PS) to NCI-H1944 tumor-bearing mice resulted in potent tumor growth inhibition after 4 weeks of i.v. injections at 2.0 μmol/kg every 3^{rd} day (FIG. 9E). Again, this dosage represents only 0.1× that of AZD4785, with which the pacDNA was able to achieve a comparable level of antitumor response in an identical animal model. The treatment with the pacDNAs substantially delays the time to reach the surrogate survival endpoint, as determined by the Kaplan-Meier survival analysis (FIG. 9F). Immunohistostaining staining shows an apparent reduction in KRAS protein levels (FIG. 10G). Reduced tumor cellularity was confirmed through H&E staining (FIG. 10G). Throughout the 27-day treatment period, mice body weight for both the pacDNA- and control-treated groups remained constant (FIG. 9G). Collectively, these data demonstrate that systemic delivery of pacDNAs in preclinical models of KRAS^{MUT} NSCLC can achieve potent KRAS downregulation and selective antitumor activity at a significantly lower dosage than what is previously possible, while using natural, unmodified oligonucleotides.

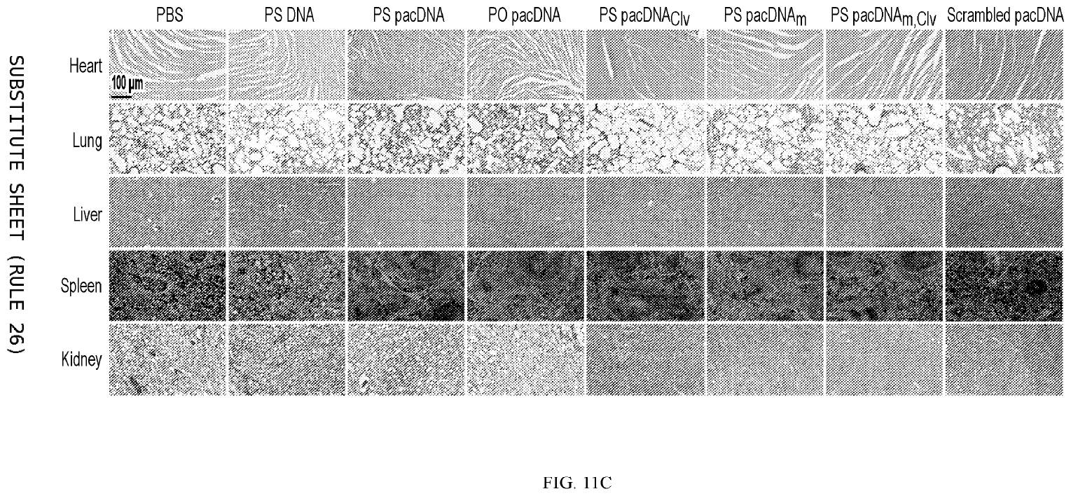

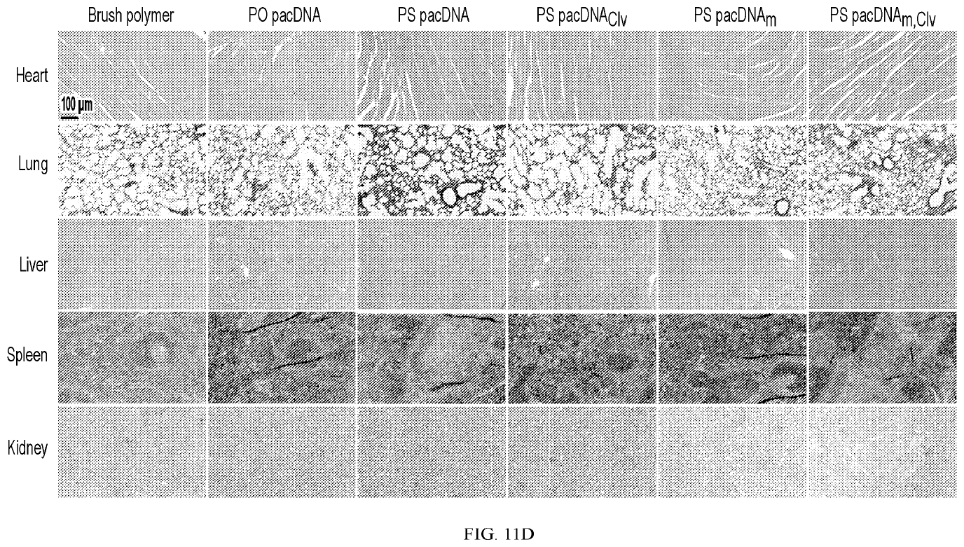

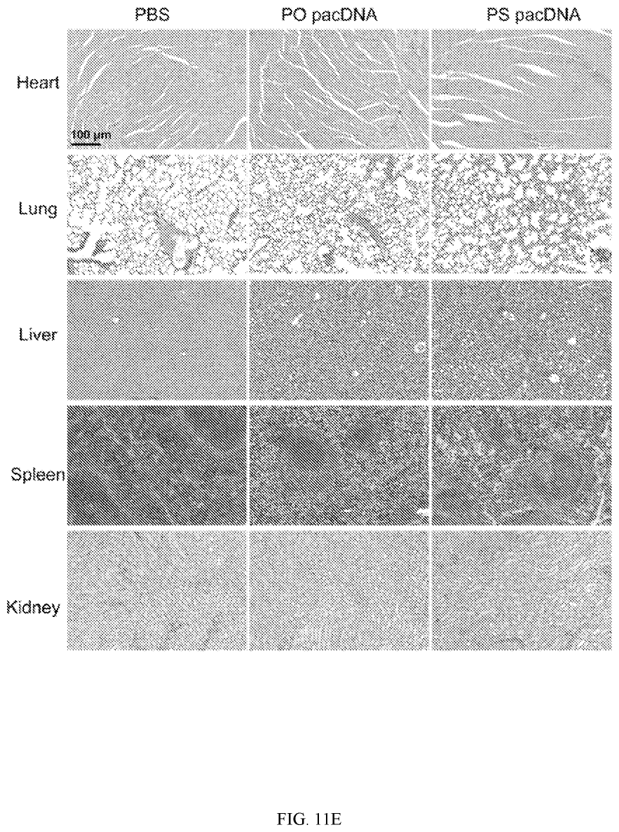

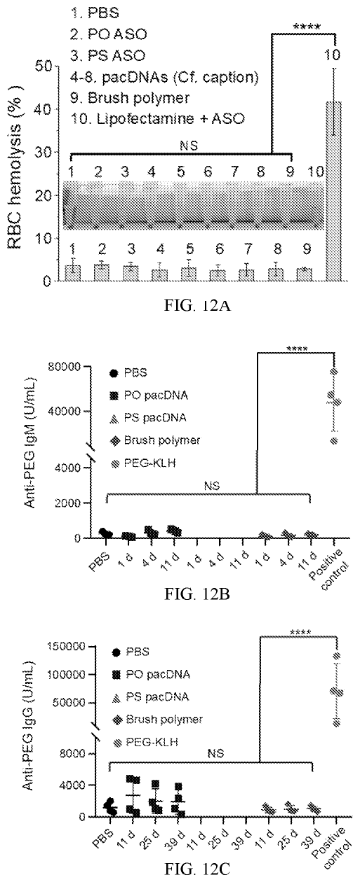

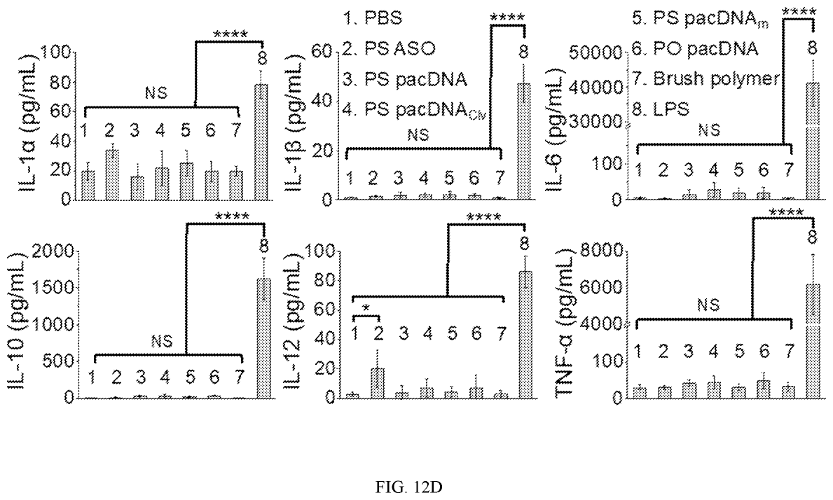

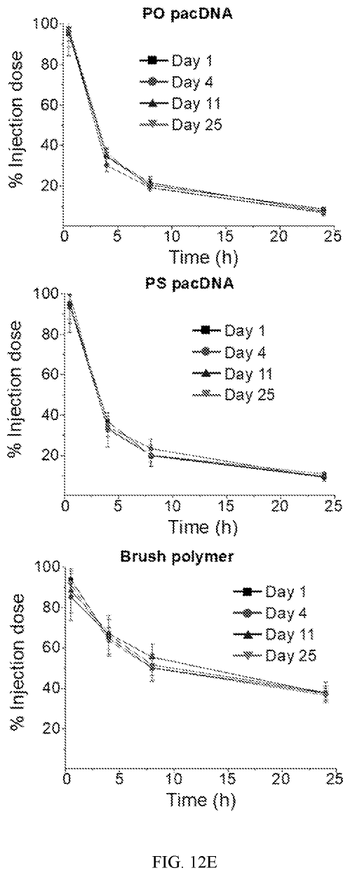

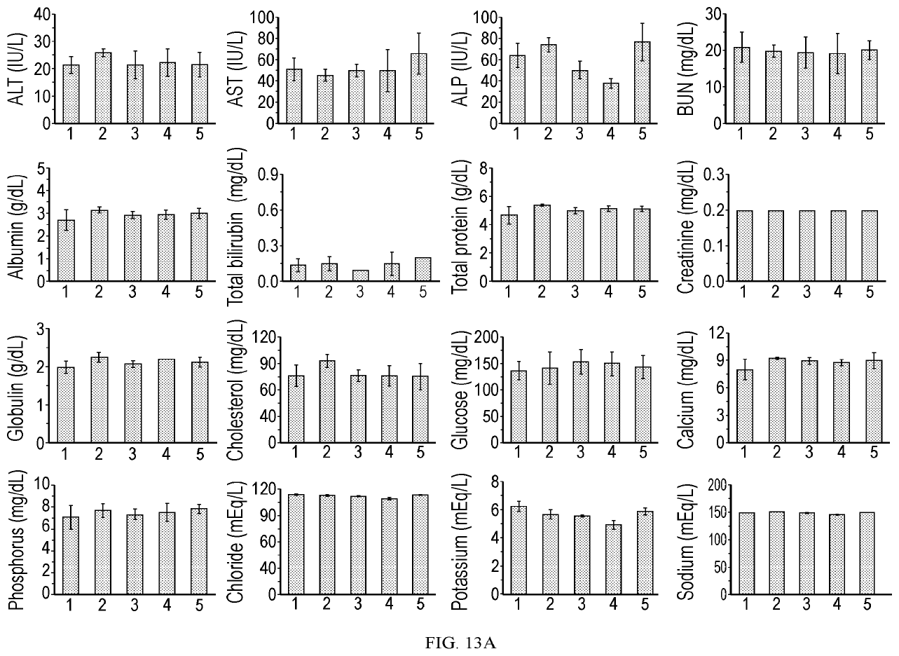

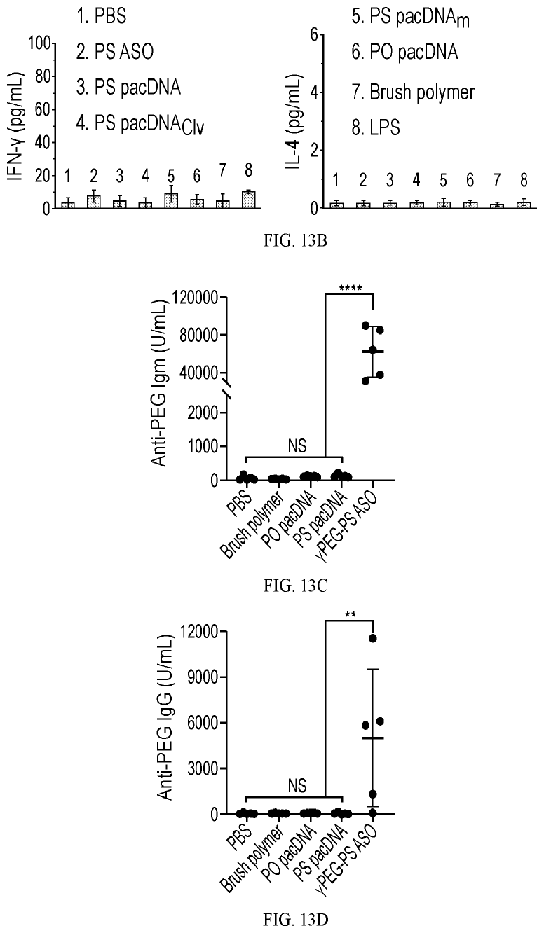

Treatment with pacDNA is well tolerated in mice without apparent body weight loss or obvious changes in behavior (refusal to eat, startle response, etc.) (FIGS. 9G, 11A, and 11B). Histological staining of major organs (heart, spleen, liver, lung, and kidneys) with H&E shows no distinct variations between pacDNA- and vehicle-treated groups (FIGS. 11C, 11D, and 11E). Oftentimes, gene vector materials (e.g., polycationic agents or surfactant-like materials such as micelles and liposomes) exhibit varying degrees of blood incompatibility, such as hemagglutination or hemolysis. The pacDNA, being non-cationic and non-self-assembled, does not display noticeable hemolytic activity, as estimated by measuring the amount of the hemoglobin released from red blood cells (RBCs) under physiological conditions (FIG. 12A). For comparison, Lipofectamine 2k, a commercially available transfection agent, resulted in ˜42% hemolysis to deliver an equivalent amount of ASO. In addition, liver indicators, including alanine aminotransferase (ALT), alkaline phosphatase (ALP), aspartate aminotransferase (AST), albumin, total bilirubin, and total protein, show no hepatic dysfunction associated with pacDNA (FIG. 13A). Renal function indexes (urea nitrogen and creatinine) as well as hematological parameters (globulin, cholesterol, glucose, calcium, phosphorus, chloride, potassium, sodium, and hemolysis and lipemia indices) are within normal ranges.

Unintended activation of the immune system was investigated in C57BL/6 mice following i.v. delivery of pacDNAs. Cytokines related to the innate and adaptive immunity (FIGS. 12D and 13B), such as tumor necrosis factor-α (TNF-α), interferon gamma (IFNγ), interleukin 1 alpha (IL-1α), IL-1β, IL-4, IL-6, IL-10, and IL-12 show no obvious changes as determined by enzyme-linked immunosorbent assays (ELISA). In contrast, lipopolysaccharide (LPS, positive control), induced strong expression of the majority of these cytokines. The anti-carrier adaptive immunity following repeated dosages can be a significant difficulty for biopharmaceutical development, even with weakly antigenic carrier materials such as PEG, which leads to the accelerated blood clearance (ABC) phenomenon and increased hepatic/splenic accumulation. Rodent as well as large animal studies have illustrated that anti-PEG antibodies abolish the extended circulation times that PEG generally provides to conjugated therapeutics. Anti-PEG immunity may also result in serious complications beyond poor plasma PK, including hypersensitivity reactions, which can lead to anaphylaxis and death. To evaluate the anti-PEG IgM/IgG response and the potential ABC effect, repeated i.v. injections of pacDNAs (PO and PS) and free bottlebrush polymer were performed on healthy C57BL/6 mice at a dose of 0.5 μmol/kg (injections on the 1^{st}, 4^{th}, 11^{th}, and the 25^{th} day). The PS pacDNA induced a very limited anti-PEG IgM response, as measured on days 4 and 11 (FIG. 12B), whereas both pacDNA forms (but not the free polymer) produced an above-baseline level of IgG responses after 11 days (FIG. 12C). Both responses, however, are extremely weak compared to a positive control (PEG-keyhole limpet hemocyanin [KLH] conjugate). Indeed, these anti-PEG antibody levels are insufficient to cause noticeable changes in plasma PK in subsequent injections of the pacDNA. As shown in FIG. 12E, blood clearance profiles of pacDNAs and the free bottlebrush polymer on days 1, 4, 11, and 25 are essentially identical. Remarkably, the low PEG antigenicity appears to be unique to the pacDNA structure. When pacDNAs (both PO and PS), brush polymer, or _{Y}PEG-PS ASO were regularly given i.v. to C57BL/6 mice (12 injections over 36 days, 0.5 μmol/kg), the _{Y}PEG-PS ASO-treated group developed very high IgM and IgG antibody titers 7 and 14 days after the last injection, respectively, but no apparent anti-PEG antibodies were detected in the pacDNA- and brush polymer-treated groups (FIGS. 13C and 13D). Taken together, these results suggest that the pacDNAs are well tolerated in mice, and the platform is generally safe without significant acute toxic and immunogenic shortcomings.

### Example 5. Bottlebrush Polymer-Locked Nucleic Acid (pacLNA) Conjugates

AZD4785 sequence is adopted in this example (Table 4), which targets the 3′ untranslated region (3′ UTR) of the KRAS mRNA and shows selective efficacy in KRAS^{MUT} cell lines. A preclinical study of AZD4785 with cEt modifications exhibits potency in treating several KRAS-dependent mutant xenografts. The same sequence of AZD4785 is chosen in this example, and synthesized in full LNA modification with a phosphodiester backbone (PO LNA) and a phosphorothioate backbone (PS LNA). The therapeutic efficacy of pacLNA was compared with the existing study of AZD4785.

TABLE 4
Sequences used in this example.

| Strand | Sequence |
|---|---|
| DBCO-modified PO LNA | 5′-DBCO-GCTATTAGGAGTCTTT-3′ |
| | (SEQ ID NO: 1) |
| DBCO-modified PS LNA | 5′-DBCO-G*C*T*A*T*T*A*G*G*A*G*T*C*T*T*T-3′ |
| | (SEQ ID NO: 7) |
| Fluorescein-labeled | 5′-DBCO-GCTATTAGGAGTCTTT-FL-3′ |
| DBCO-modified PO LNA | (SEQ ID NO: 2) |
| Cy3-labeled and DBCO- | 5′-DBCO-GCTATTAGGAGTCTTT-Cy3-3′ |
| modified PO LNA | (SEQ ID NO: 3) |
| Cy5-labeled and DBCO- | 5′-DBCO-GCTATTAGGAGTCTTT-Cy5-3′ |
| modified PO LNA | (SEQ ID NO: 4) |
| Fluorescein-labeled | 5′-DBCO-G*C*T*A*T*T*A*G*G*A*G*T*C*T*T*T*-FL-3′ |
| DBCO-modified PS LNA | (SEQ ID NO: 9) |
| Cy3-labeled and DBCO- | 5′-DBCO-G*C*T*A*T*T*A*G*G*A*G*T*C*T*T*T*-Cy3-3′ |
| modified PS LNA | (SEQ ID NO: 10) |
| Cy5-labeled and DBCO- | 5′-DBCO-G*C*T*A*T*T*A*G*G*A*G*T*C*T*T*T*-Cy5-3′ |
| modified PS LNA | (SEQ ID NO: 11) |
| Dabcyl-labeled sense | 5′-Dabcyl-AAAGACTCCTAATAGC-3′ |
| | (SEQ ID NO: 5) |
| Dabcyl-labeled scrambled | 5′-Dabcyl-ACGACTAGTATCACAA-3′ |
| sense | (SEQ ID NO: 6) |
| DBCO-modified | 5′-DBCO-GGCTACTACGCCGTCA-3′ |
| scrambled PO LNA | (SEQ ID NO: 21) |

Simple letters: DNA; italicized letters: locked nucleic acid (LNA) bases; FL: fluorescein; asterisk (*): phosphorothioate internucleotide linkage.

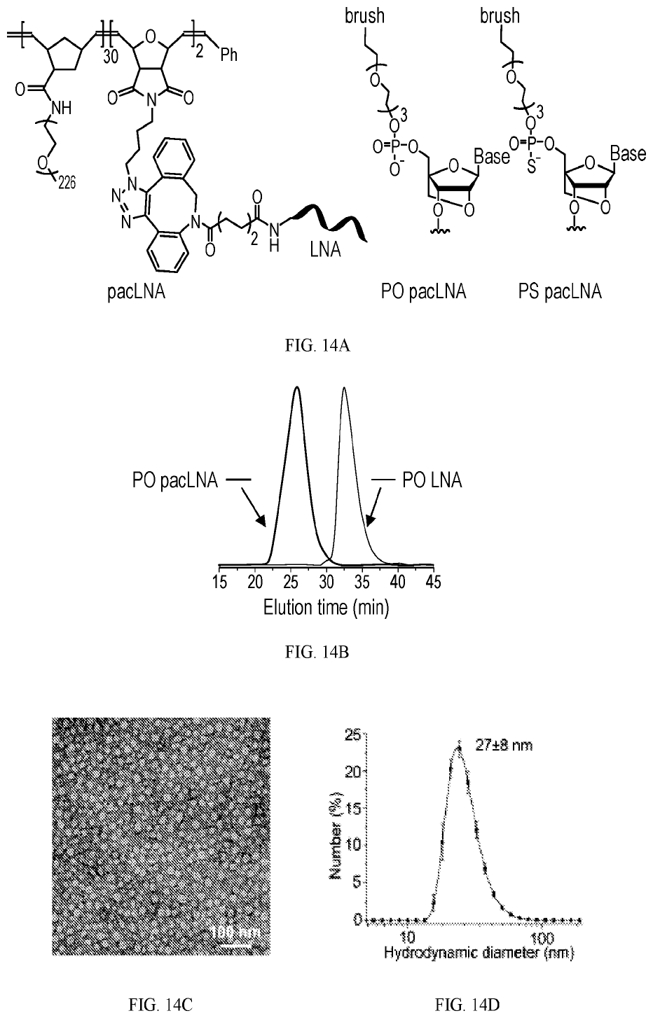

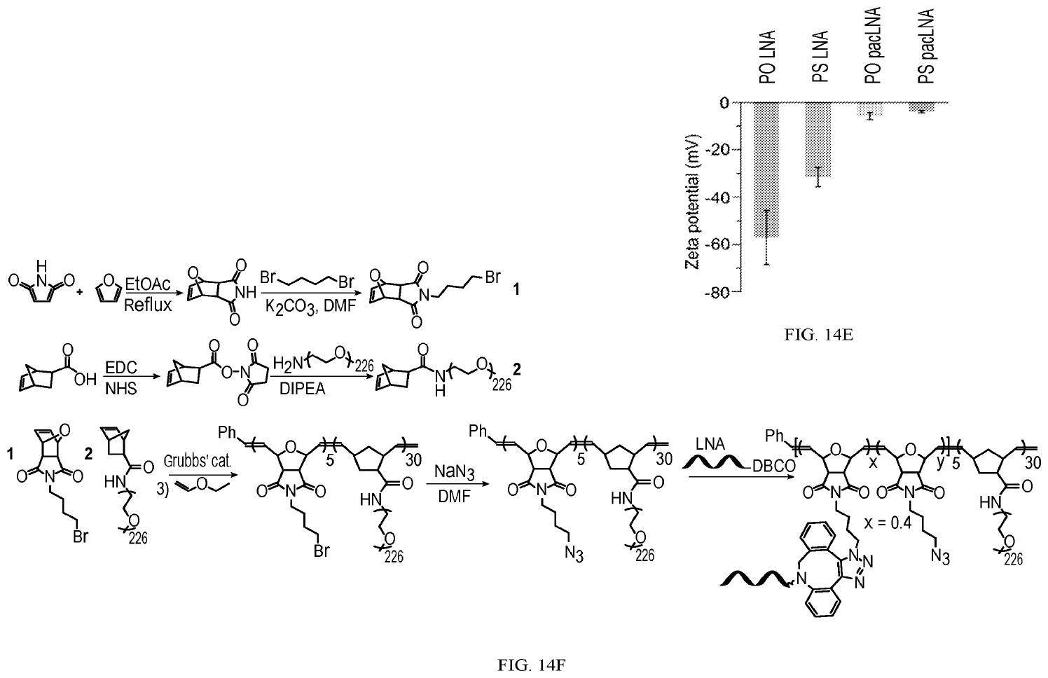

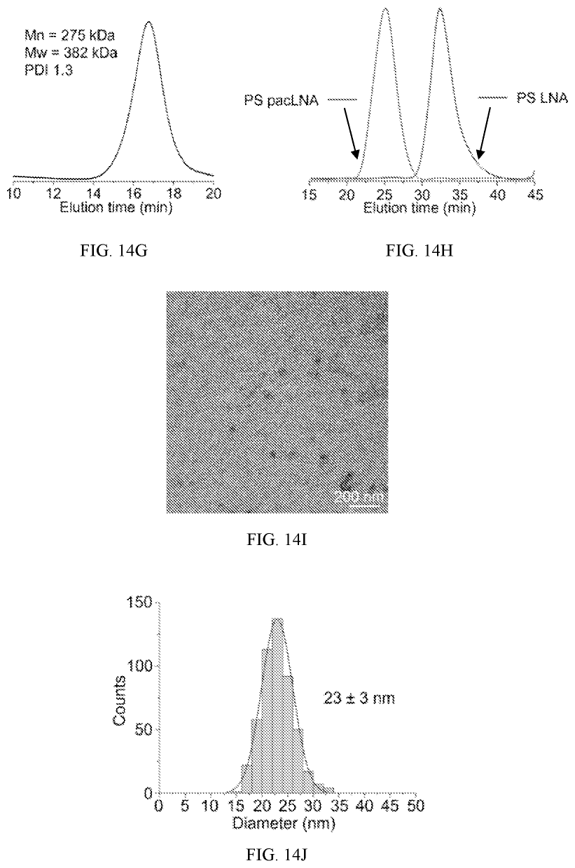

To achieve the prototypic physiochemical and biopharmaceutical characteristics of pacLNA, the bottlebrush polymer needs to be synthesized with sufficiently dense side chains and desired molecular weight to shield LNA and bypass the renal clearance. Via ring-opening metathesis polymerization (ROMP), norbornenyl-modified PEG (10 kDa, NPEG) and 7-oxanorbornenyl-bromide (ONBr) are polymerized sequentially in the ratio of 30:5, which yields a diblock bottlebrush architecture (pONBr₅-b-pNPEG₃₀, FIGS. 14A and 14F) with an average molecular weight (Mn) ˜300 kDa and a polydispersity index (PDI) ˜1.3, as determined by N,N-dimethylformamide gel permeation chromatography (DMF-GPC, FIG. 14G). After purification, dibenzocyclooctyne (DBCO)-modified PO and PS LNA strands are conjugated to the azide-functionalized bottlebrush polymer to yield pacLNA. The average number of LNA per brush is 2˜3. The successful conjugation of pacLNA is confirmed by aqueous GPC, which shows a narrow dispersity for pacLNA and a baseline separation between pacLNAs and free LNAs (FIGS. 14B and 14H). The hydrodynamic size of pacLNA is 27±8 nm as measured by dynamic light scattering (FIG. 14D). Transmission electron microscopy reveals a slightly smaller size distribution of pacLNA, 23±3 nm and shows a spherical morphology in their dry state (FIGS. 14C, 14I, and 14J). ζ potential measurements indicate that pacLNAs in Nanopure™ water have a slight negative charge (−5 ˜−3 mV), which is largely below the ζ potential of PO LNA, −57 mV and PS LNA, −32 mV (FIG. 14E).

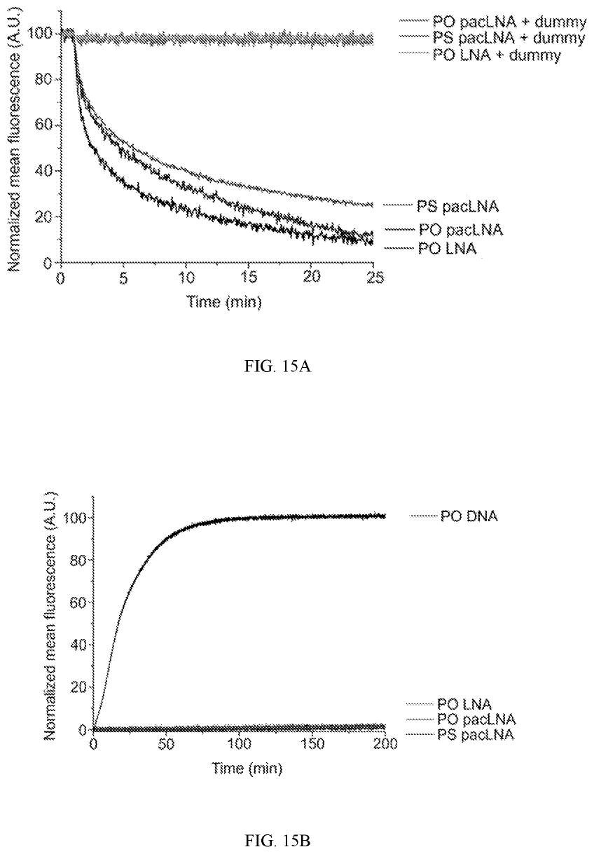

pacLNA is designed to reduce unwanted oligonucleotide-protein interactions, and protect the LNA from being degraded but remain its hybridizing ability to the complementary strand. To test the hybridizing kinetics and the nuclease degradation kinetics of pacLNA. LNAs and pacLNAs labeled with a fluorophore on its 3′ position were examined. 5′-quencher labeled complementary and dummy strands are added to the fluorescein-labeled pacLNA. Hybridization results in the quenching of the fluorescein label, and a decrease of the fluorescein signal. The results show that LNA-modified ASO has a slightly slow hybridization rate compared to DNA, reach to ˜80% completion in 10 min. After conjugated with bottlebrush polymer, both PO pacLNA and PS pacLNA show a similar hybridizing rate as PO LNA, indicating that the bottlebrush polymer does not interfere with the hybridization (FIG. 15A). Adding the 5′-dabcyl labeled dummy strand does not result in a decrease in fluorescence signal, excluding the non-specific hybridization. Then, the hybridized PO LNA, PO, and PS pacLNAs were treated with DNase I, which is an endonuclease recognizing and digesting double-stranded DNA. 0.2 U/mL of DNase I quickly cleaves PO DNA with a t_{1/2}≈11 min as indicated by an increase of fluorescence intensity. Treating LNA-DNA duplex with DNase I at the same concentration does not result in a rapid increase of fluorescence signals, indicating that LNA-DNA duplex can resist DNase I degradation and remain intact for several hours. PO and PS pacLNAs are as stable as the LNA towards DNase I degradation, which are hardly degraded in 200 min (FIG. 15B). Conclusively, pacLNAs exhibit high stability towards nuclease degradation, and remain moderate capability to hybridize with their target.

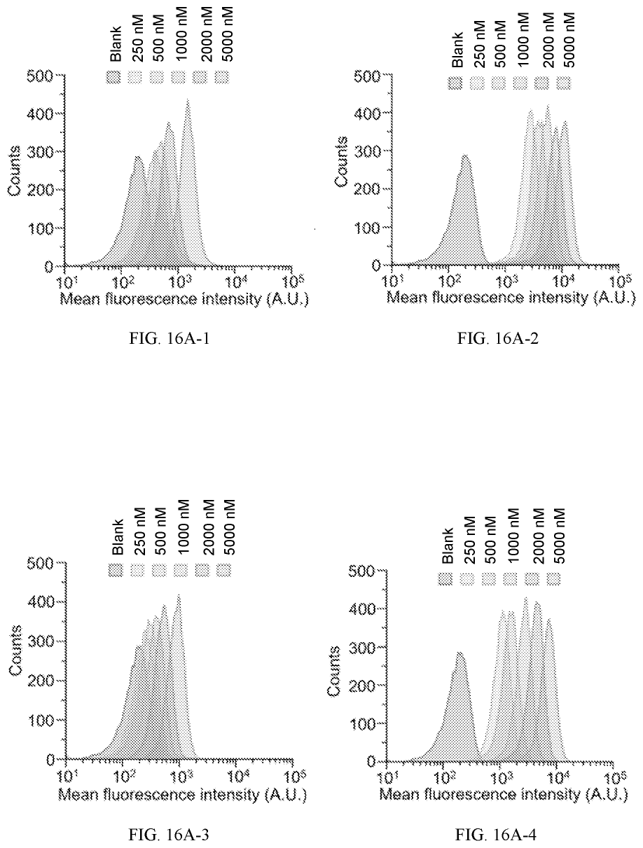

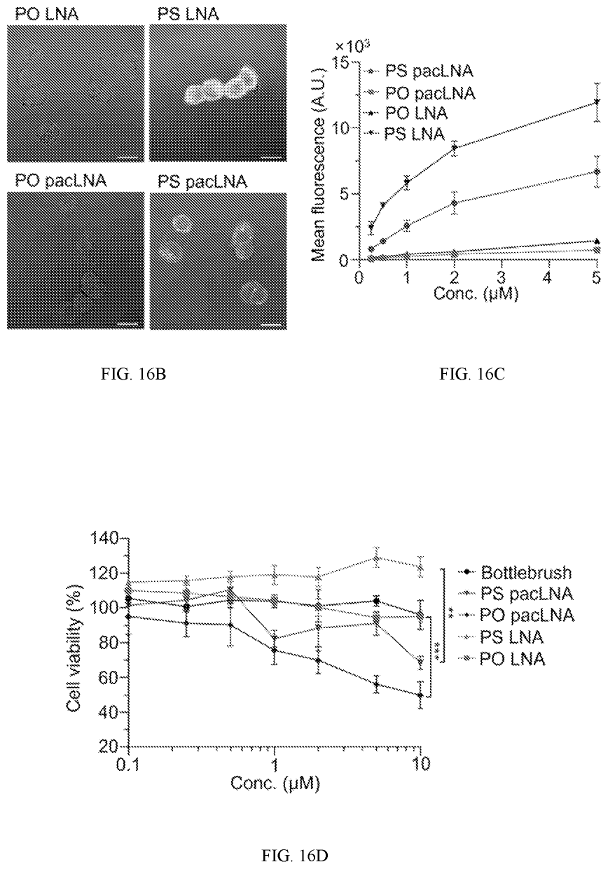

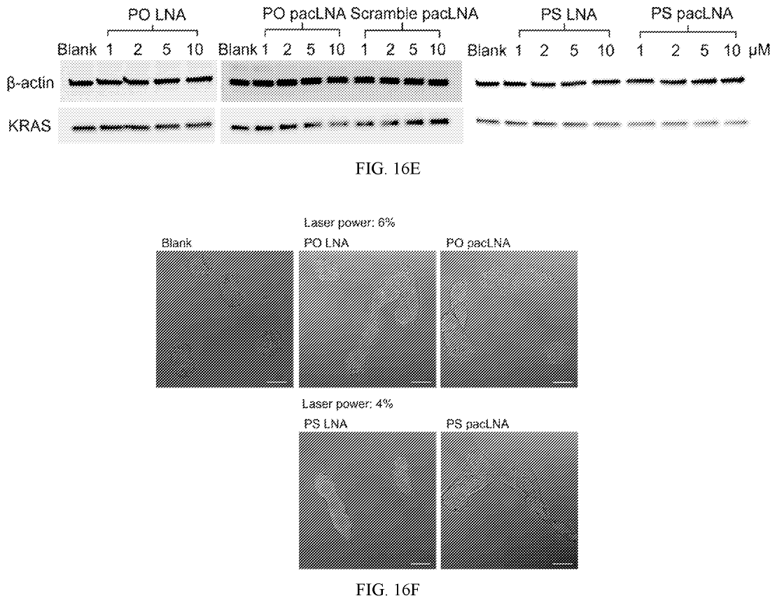

Next, to investigate the in vitro efficacy of pacLNA. Cellular uptake studies were performed using Cyanine 3 (Cy3) labeled free LNAs and pacLNAs. NCI-H358 cells were treated with Cy3-labeled samples in serum-free media for 4 h, then analyzed by flow cytometry. The results show that LNA modifications boost the cellular uptake of ASO (FIG. 16A). PS LNA shows the fastest cellular uptake rate among the samples, which is facilitated by PS LNA-protein interaction. Conjugation to the brush polymer results in a moderate internalization rate of PO pacLNA (0.52× of PO LNA) and PS pacLNA (0.62× of PS LNA, FIG. 16C). This result is compatible with the hypothesis that bottlebrush polymer would shield LNA and hinder its interaction with extracellular or membrane protein. Confocal microscopy also confirms that pacLNAs successfully enter the cells (FIGS. 16B and 16F). Then, the knockdown efficacy of KRAS protein in NCI-H358 cells after treatment with pacLNA and controls at concentrations from 1 to 10 μM was investigated. pacLNAs show a dose-dependent knock down of KRAS protein, whereas bottlebrush polymer carrying a scramble sequence does not reduce KRAS protein level, which rules out the nonspecific effects. PO and PS LNAs although exhibit higher cellular uptake level, they do not lead to antisense activity in vitro (FIG. 16E), which is attributed to the insufficient steric blocking of free LNAs. A prior study reveals that the mechanism of pacDNA is steric blocking regardless the types of chemical modifications. LNA modification alone is not sufficient to block the translation of ribosome, whereas bottlebrush polymer can facilitate the steric blocking effect. Then the cell viability of NCI-H358 through a 3-(4,5-dimethylthiazol-2-yl)-2,5 diphenyl tetrazolium bromide (MTT) assay by treating cells with pacLNAs and controls for 48 h was analyzed. The results show that PO pacLNA and PS pacLNA inhibit the cell growth by 40% and 30%, respectively. The control groups, including brush polymer, PO and PS LNA do not exhibit any significant changes in cell viability (FIG. 16D), which is consistent with the western blotting results. Through in vitro studies, pacLNAs exhibit moderate cellular uptake, efficient internalization, and antisense activity towards NCI-H358 cell line.

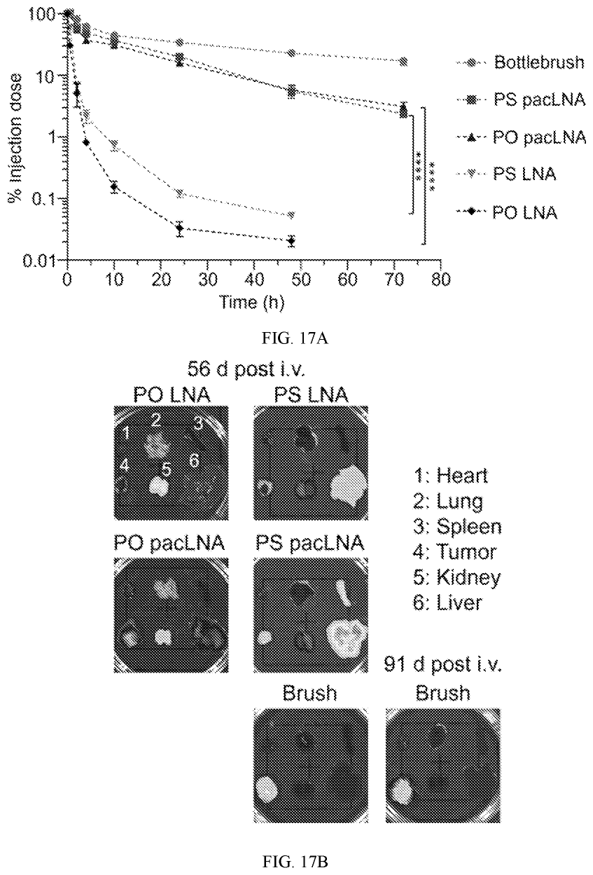

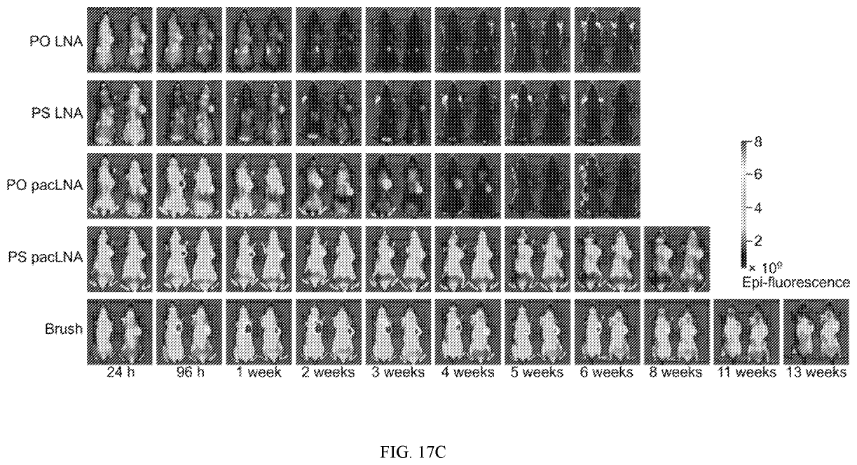

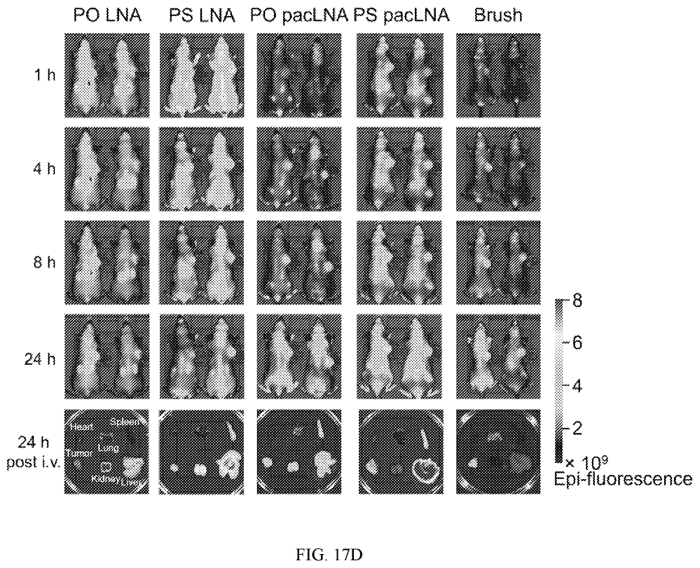

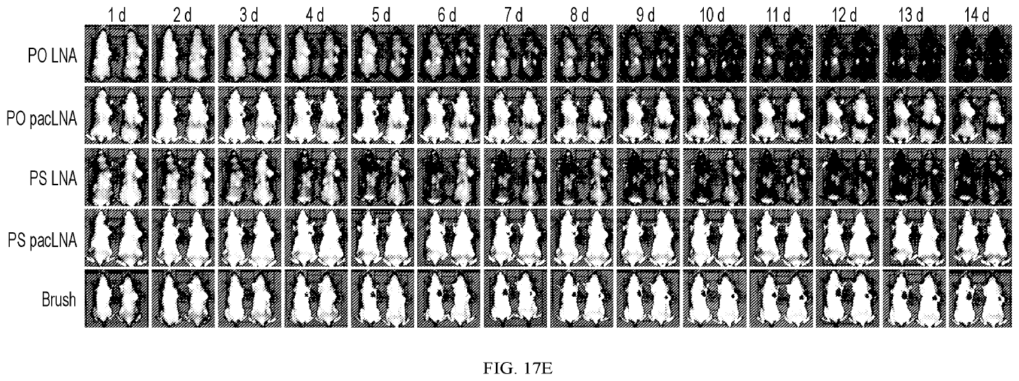

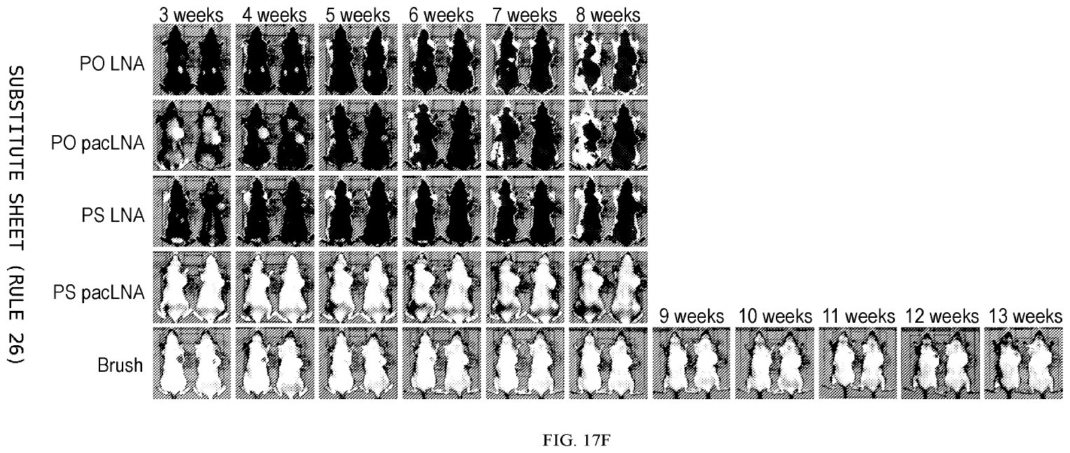

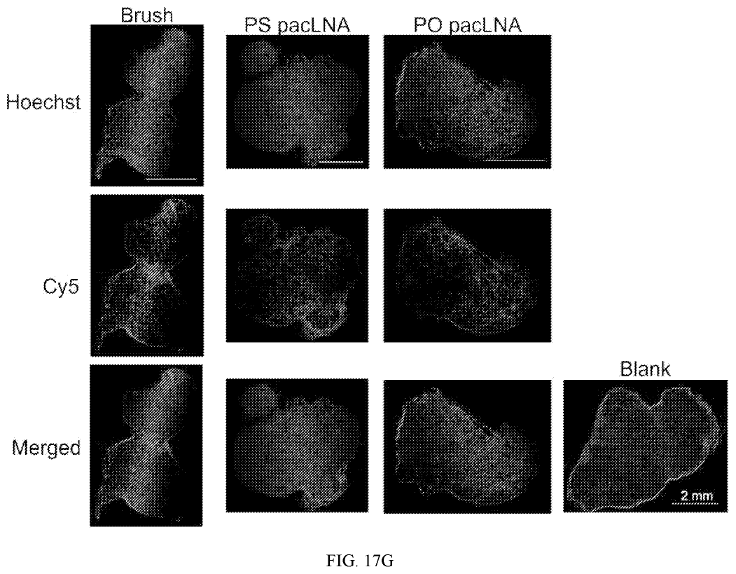

Efficient delivery and biodistribution underlay the in vivo potency of pacLNA. To investigate the pharmacokinetic properties of pacLNAs, the Cy5-labeled LNAs and pacLNAs were intravenously injected into C57BL/6 mice, and the blood was collected at predetermined time points in 72 h. The plasma was separated and the Cy5 fluorescence intensity was measured using a plate reader. The results show that LNAs, although fully modified, undergo rapid clearance and have short elimination half-lives (t_{1/2β,PO}=4.06 h, t_{1/2β, PS}=4.17 h; two-compartment model, FIG. 17A and Table 5). ˜ 60% of PS LNA and ˜30% of PO LNA remained in blood circulations in 30 min, then both quickly reduced to <5% in 2 h. In contrast, PO and PS pacLNA persist in blood for much longer time, with ˜20% of PS pacLNA and 16% of PO pacLNA circulating in the blood 24 h post i.v., and exhibit 3 times longer elimination half-lives compared with free LNA (t_{1/2β,PO pacLNA}=14.6 h, t_{1/2β, PS pacLNA}=13.8 h). Bottlebrush polymer shows an astonishing PK property. ˜35% of bottlebrush polymer circulates in blood after 24 h, and with ˜17% remaining after 72 h. The enhanced PK properties elevate the bioavailability of pacLNAs as indicated by the area under the curve (AUC_{∞}) which shows an improvement of 13-18 times compared with free LNAs (Table 5). Collectively, pacLNAs exhibit higher blood concentrations and prolonged circulation times due to the efficient shielding of bottlebrush polymer.

TABLE 5
Plasma pharmacokinetic parameters of free LNAs,
pacLNAs and bottlebrush polymer in C57BL/6 mice.

| | | | AUC_{∞} |
|---|---|---|---|
| Sample | t_{1/2α} (h) | t_{1/2β} (h) | (nmol/ml · h) |
| PO LNA | 0.57 (±0.01) | 4.06 (±0.12) | 5.06 (±0.25) |
| PS LNA | 0.40 (±0.01) | 4.17 (±0.13) | 8.37 (±0.30) |
| PO pacLNA | 0.67 (±0.22) | 14.64 (±2.18) | 92.22 (±2.85) |
| PS pacLNA | 0.40 (±0.29) | 13.84 (±1.31) | 108.24 (±5.77) |
| Bottlebrush polymer | 1.83 (±0.14) | 28.96 (±4.38) | 167.76 (±5.58) |

Prolonged blood circulation times and higher bioavailability lead to access and retention at tumor sites. To investigate the biodistribution of pacLNAs, live mice fluorescence monitoring using IVIS for NCI-H358 tumor-bearing athymic mice was performed. Cy5-labeled LNAs and pacLNAs were injected intravenously. Mice were monitored at predetermined time points, daily and weekly. Both free LNAs and pacLNAs exhibited durable fluorescence signals in live mice 24 h post i.v. (FIG. 17D). Fluorescence images of dissected organs confirm the accumulation of free LNAs in kidneys and liver. Interestingly, PS LNA exhibits access by tumor, which suggests that the LNA conformation inhibit the recognition of PS backbone by proteins. Therefore, PS LNA would experience a relatively slow clearance. pacLNAs showed accumulations in tumor in live mice and organs through fluorescence imaging. The long-term live mice fluorescence imaging results show that LNA modifications are stable and can accumulate for a longer time at tumor sites compared to DNA. The fluorescence signal diminishes after one week for PO LNA and two weeks for PS LNA (FIGS. 17C and 17E). pacLNAs exhibit a much stronger retention at tumor sites after single injection. The peak of pacLNAs were achieved after 96 h and remained detectable till 4 weeks for PO pacLNA and 8 weeks for PS pacLNA (FIG. 17F). The bottlebrush polymer alone exhibits the longest time of accumulation at tumors. The fluorescence signal remained detectable after 13 weeks. Fluorescence images of dissected organs 56 d post i.v. reveal the accumulation of pacLNAs and bottlebrush polymer in tumor, whereas free LNAs end up in kidney (PO LNA) and liver (PS LNA, FIG. 17B). The confocal microscopy of cryosectioned tumor splices confirms the penetration of pacLNAs into the solid tumor (FIG. 17G). Collectively, these data indicate that pacLNAs exhibit high accumulation by non-liver organs and long retention at tumor site after one single injection.

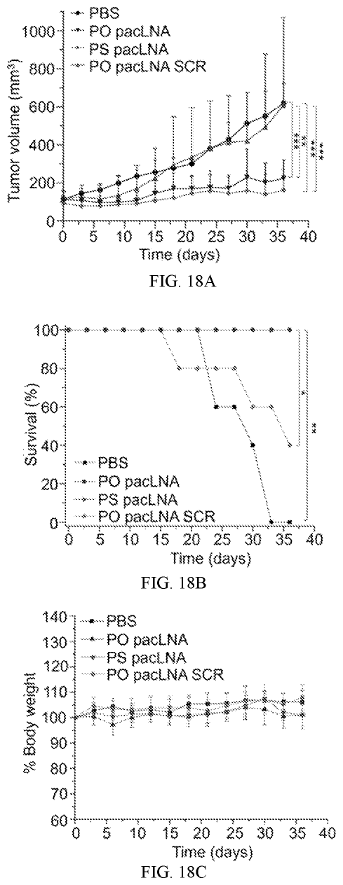

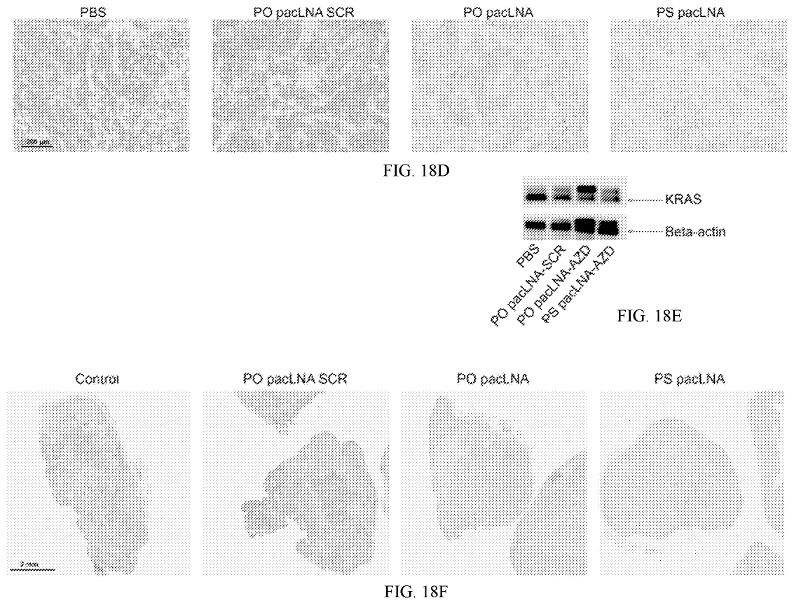

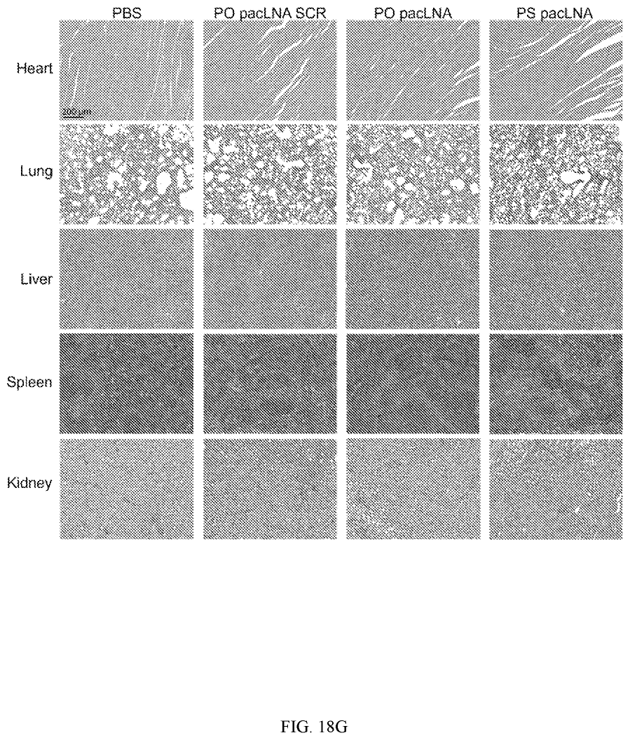

With enhanced biopharmaceutical properties, the in vivo efficacy of pacLNA at a weekly dosage in female athymic mice bearing NCI-H358 xenografts was tested. pacLNAs and vehicles were administrated intravenously to the mice when the tumor volume reaches 100 mm³. 0.5 μmol/kg of pacLNAs was given to mice once a week for a total of 5 doses. After 5 treatments, the tumor growth of mice in pacLNA groups were significantly inhibited with an average of tumor volume at 160˜220 mm³ (FIG. 18A). Bottlebrush polymer carrying a scramble sequence and vehicle treated groups show tumor volumes around 600 mm³, which rules out non-specific effect of pacLNA. Tumor volumes were suppressed by ⅓ of the treated groups with a total dosage of 50 nmole of pacLNA. Comparing with the existing study of AZD4785, the total dosage was lowered to 1%. According to Kaplan-Meier survival analysis, pacLNAs treated groups exhibit longer survival time towards surrogate endpoint (FIG. 18B). Immunohistochemistry staining of tumors verifies that pacLNAs reduce KRAS protein level after five treatments in 36 days (FIGS. 18D, 18E, and 18F). Mice treated with pacLNAs do not exhibit apparent body weight loss or obvious changes in behavior (FIG. 18C). Histological staining of major organs (heart, spleen, liver, lung, and kidney) with hematoxylin and eosin (H&E) shows no distinct variations between pacLNA-treated and control groups (FIG. 18G). These results suggest that pacLNAs are highly efficient and well-tolerated in mice.

### REFERENCES

Bajan, S., & Hutvagner, G. (2020). RNA-based therapeutics: from antisense oligonucleotides to miRNAs. Cells, 9 (1), 137.
Balijepalli, A., Robertson, J. W., Reiner, J. E., Kasianowicz, J. J., & Pastor, R. W. (2013). Theory of polymer-nanopore interactions refined using molecular dynamics simulations. Journal of the American Chemical Society, 135 (18), 7064-7072.
Berendsen, H. J., Postma, J. V., Van Gunsteren, W. F., DiNola, A. R. H. J., & Haak, J. R. (1984). Molecular dynamics with coupling to an external bath. The Journal of chemical physics, 81 (8), 3684-3690.
Cao, X., Lu, X., Wang, D., Jia, F., Tan, X., Corley, M., . . . & Zhang, K. (2017). Modulating the Cellular Immune Response of Oligonucleotides by Brush Polymer-Assisted Compaction. Small, 13 (43), 1701432.
Chin, L., Tam, A., Pomerantz, J., Wong, M., Holash, J., Bardeesy, N., . . . & Yancopoulos, G. D. (1999). Essential role for oncogenic Ras in tumour maintenance. Nature, 400 (6743), 468-472.
Chiriboga, C. A., Swoboda, K. J., Darras, B. T., Iannaccone, S. T., Montes, J., Darryl, C., . . . & Bishop, K. M. (2016). Results from a phase 1 study of nusinersen (ISIS-SMNRx) in children with spinal muscular atrophy. Neurology, 86 (10), 890-897.
Choi, C. H. J., Hao, L., Narayan, S. P., Auyeung, E., & Mirkin, C. A. (2013). Mechanism for the endocytosis of spherical nucleic acid nanoparticle conjugates. Proceedings of the National Academy of Sciences, 110 (19), 7625-7630.
Cox, A. D., Fesik, S. W., Kimmelman, A. C., Luo, J., & Der, C. J. (2014). Drugging the undruggable RAS: Mission possible?. Nature reviews Drug discovery, 13 (11), 828-851.
Crooke, S. T., Baker, B. F., Crooke, R. M., & Liang, X. H. (2021). Antisense technology: an overview and prospectus. Nature Reviews Drug Discovery, 20 (6), 427-453.
Crooke, S. T., Seth, P. P., Vickers, T. A., & Liang, X. H. (2020). The interaction of phosphorothioate-containing RNA targeted drugs with proteins is a critical determinant of the therapeutic effects of these agents. Journal of the American Chemical Society, 142 (35), 14754-14771.
Crooke, S. T., Vickers, T. A., & Liang, X. H. (2020). Phosphorothioate modified oligonucleotide-protein interactions. Nucleic acids research, 48 (10), 5235-5253.
Crooke, S. T., Wang, S., Vickers, T. A., Shen, W., & Liang, X. H. (2017). Cellular uptake and trafficking of antisense oligonucleotides. Nature biotechnology, 35 (3), 230-237.
Ding, H., Li, J., Chen, N., Hu, X., Yang, X., Guo, L., . . . & Fan, C. (2018). DNA nanostructure-programmed like-charge attraction at the cell-membrane interface. ACS central science, 4 (10), 1344-1351.
Eckstein, F. (2014). Phosphorothioates, essential components of therapeutic oligonucleotides. Nucleic acid therapeutics, 24 (6), 374-387.
Fisher, G. H., Wellen, S. L., Klimstra, D., Lenczowski, J. M., Tichelaar, J. W., Lizak, M. J., . . . & Varmus, H. E. (2001). Induction and apoptotic regression of lung adenocarcinomas by regulation of a K-Ras transgene in the presence and absence of tumor suppressor genes. Genes & development, 15 (24), 3249-3262.
Gagliardi, M., & Ashizawa, A. T. (2021). The challenges and strategies of antisense oligonucleotide drug delivery. Biomedicines, 9 (4), 433.
Geary, R. S., Norris, D., Yu, R., & Bennett, C. F. (2015). Pharmacokinetics, biodistribution and cell uptake of antisense oligonucleotides. Advanced drug delivery reviews, 87, 46-51.
Graham, M. J., Crooke, S. T., Monteith, D. K., Cooper, S. R., Lemonidis, K. M., Stecker, K. K., . . . & Crooke, R. M. (1998). In vivo distribution and metabolism of a phosphorothioate oligonucleotide within rat liver after intravenous administration. Journal of Pharmacology and Experimental Therapeutics, 286 (1), 447-458.
Hammond, S. M., Aartsma-Rus, A., Alves, S., Borgos, S. E., Buijsen, R. A., Collin, R. W., . . . & Arechavala-Gomeza, V. (2021). Delivery of oligonucleotide-based therapeutics: challenges and opportunities. EMBO molecular medicine, 13 (4), e13243.
Harris, J. M., & Chess, R. B. (2003). Effect of pegylation on pharmaceuticals. Nature reviews Drug discovery, 2 (3), 214-221.
Henry, S. P., Bolte, H., Auletta, C., & Kornbrust, D. J. (1997). Evaluation of the toxicity of ISIS 2302, a phosphorothioate oligonucleotide, in a four-week study in cynomolgus monkeys. Toxicology, 120 (2), 145-155.
Henry, S. P., Novotny, W., Leeds, J., Auletta, C., & Kornbrust, D. J. (1997). Inhibition of coagulation by a phosphorothioate oligonucleotide. Antisense and Nucleic Acid Drug Development, 7 (5), 503-510.
Herkt, M., & Thum, T. (2021). Pharmacokinetics and proceedings in clinical application of nucleic acid therapeutics. Molecular Therapy, 29 (2), 521-539.
Hovingh, K., Besseling, J., & Kastelein, J. (2013). Efficacy and safety of mipomersen sodium (Kynamro). Expert opinion on drug safety, 12 (4), 569-579.
Jia, F., Lu, X., Tan, X., Wang, D., Cao, X., & Zhang, K. (2017). Effect of PEG architecture on the hybridization thermodynamics and protein accessibility of PEGylated oligonucleotides. Angewandte Chemie, 129 (5), 1259-1263.
Jia, F., Lu, X., Wang, D., Cao, X., Tan, X., Lu, H., & Zhang, K. (2017). Depth-profiling the nuclease stability and the gene silencing efficacy of brush-architectured poly (ethylene glycol)-DNA conjugates. Journal of the American Chemical Society, 139 (31), 10605-10608.
Jia, F., Wang, D., Lu, X., Tan, X., Wang, Y., Lu, H., & Zhang, K. (2018). Improving the enzymatic stability and the pharmacokinetics of oligonucleotides via DNA-backboned bottlebrush polymers. Nano letters, 18 (11), 7378-7382.
Juliano, R. L. (2016). The delivery of therapeutic oligonucleotides. Nucleic acids research, 44 (14), 6518-6548.
Juliano, R., Alam, M. R., Dixit, V., & Kang, H. (2008). Mechanisms and strategies for effective delivery of antisense and siRNA oligonucleotides. Nucleic acids research, 36 (12), 4158-4171.
Kamerkar, S., LeBleu, V. S., Sugimoto, H., Yang, S., Ruivo, C. F., Melo, S. A., . . . & Kalluri, R. (2017). Exosomes facilitate therapeutic targeting of oncogenic KRAS in pancreatic cancer. Nature, 546 (7659), 498-503.
Khvorova, A., & Watts, J. K. (2017). The chemical evolution of oligonucleotide therapies of clinical utility. Nature biotechnology, 35 (3), 238-248.
Koller, E., Vincent, T. M., Chappell, A., De, S., Manoharan, M., & Bennett, C. F. (2011). Mechanisms of single-stranded phosphorothioate modified antisense oligonucleotide accumulation in hepatocytes. Nucleic acids research, 39 (11), 4795-4807.
Kozma, G. T., Shimizu, T., Ishida, T., & Szebeni, J. (2020). Anti-PEG antibodies: Properties, formation, testing and role in adverse immune reactions to PEGylated nano-biopharmaceuticals. Advanced drug delivery reviews, 154, 163-175.
Lächelt, U., & Wagner, E. (2015). Nucleic acid therapeutics using polyplexes: a journey of 50 years (and beyond). Chemical reviews, 115 (19), 11043-11078.
Lee, H., de Vries, A. H., Marrink, S. J., & Pastor, R. W. (2009). A coarse-grained model for polyethylene oxide and polyethylene glycol: conformation and hydrodynamics. The journal of physical chemistry B, 113 (40), 13186-13194.
Levin, A. A., & Henry, S. P. (2008). Toxicology of oligonucleotide therapeutics and understanding the relevance of the toxicities. Preclinical safety evaluation of biopharmaceuticals: a science-based approach to facilitating clinical trials. Hoboken (NJ): John Wiley & Sons, Inc, 537-75.
Li, H., Li, Y., Xiao, Y., Zhang, B., Cheng, Z., Shi, J., . . . & Zhang, K. (2020). Well-defined DNA-polymer miktoarm stars for enzyme-resistant nanoflares and carrier-free gene regulation. Bioconjugate Chemistry, 31 (3), 530-536.
Linnane, E., Davey, P., Zhang, P., Puri, S., Edbrooke, M., Chiarparin, E., . . . & Ross, S. J. (2019). Differential uptake, kinetics and mechanisms of intracellular trafficking of next-generation antisense oligonucleotides across human cancer cell lines. Nucleic acids research, 47 (9), 4375-4392.
Love, J. A., Morgan, J. P., Trnka, T. M., & Grubbs, R. H. (2002). A practical and highly active ruthenium-based catalyst that effects the cross metathesis of acrylonitrile. Angewandte Chemie, 114 (21), 4207-4209.
Lu, X., Jia, F., Tan, X., Wang, D., Cao, X., Zheng, J., & Zhang, K. (2016). Effective antisense gene regulation via noncationic, polyethylene glycol brushes. Journal of the American Chemical Society, 138 (29), 9097-9100.
Lu, X., Tran, T. H., Jia, F., Tan, X., Davis, S., Krishnan, S., . . . & Zhang, K. (2015). Providing oligonucleotides with steric selectivity by brush-polymer-assisted compaction. Journal of the American Chemical Society, 137 (39), 12466-12469.
Luo, D., & Saltzman, W. M. (2000). Synthetic DNA delivery systems. Nature biotechnology, 18 (1), 33-37.
Manoharan, M. (1999). 2′-Carbohydrate modifications in antisense oligonucleotide therapy: importance of conformation, configuration and conjugation. Biochimica et Biophysica Acta (BBA)-Gene Structure and Expression, 1489 (1), 117-130.
Marrink, S. J., Risselada, H. J., Yefimov, S., Tieleman, D. P., & De Vries, A. H. (2007). The MARTINI force field: coarse grained model for biomolecular simulations. The journal of physical chemistry B, 111 (27), 7812-7824.
Moore, A. R., Rosenberg, S. C., McCormick, F., & Malek, S. (2020). RAS-targeted therapies: is the undruggable drugged?. Nature Reviews Drug Discovery, 19 (8), 533-552.
Ng, E. W., Shima, D. T., Calias, P., Cunningham, E. T., Guyer, D. R., & Adamis, A. P. (2006). Pegaptanib, a targeted anti-VEGF aptamer for ocular vascular disease. Nature reviews drug discovery, 5 (2), 123-132.
O'Reilly, R. K., Joralemon, M. J., Wooley, K. L., & Hawker, C. J. (2005). Functionalization of micelles and shell cross-linked nanoparticles using click chemistry. Chemistry of materials, 17 (24), 5976-5988.
Ostrem, J. M., Peters, U., Sos, M. L., Wells, J. A., & Shokat, K. M. (2013). K-Ras (G12C) inhibitors allosterically control GTP affinity and effector interactions. Nature, 503 (7477), 548-551.
Phase, I. Dose-Escalation Study of AZD4785 in Patients with Advanced Solid Tumours.
Pontrello, J. K., Allen, M. J., Underbakke, E. S., & Kiessling, L. L. (2005). Solid-phase synthesis of polymers using the ring-opening metathesis polymerization. Journal of the American Chemical Society, 127 (42), 14536-14537.
Roberts, T. C., Langer, R., & Wood, M. J. (2020). Advances in oligonucleotide drug delivery. Nature Reviews Drug Discovery, 19 (10), 673-694.
Rosell, R., Aguilar, A., Pedraz, C., & Chaib, I. (2021). KRAS inhibitors, approved. Nature Cancer, 2 (12), 1254-1256.
Ross, S. J., Revenko, A. S., Hanson, L. L., Ellston, R., Staniszewska, A., Whalley, N., . . . & Macleod, A. R. (2017). Targeting KRAS-dependent tumors with AZD4785, a high-affinity therapeutic antisense oligonucleotide inhibitor of KRAS. Science translational medicine, 9 (394), eaal5253.
Ryan, M. B., & Corcoran, R. B. (2018). Therapeutic strategies to target RAS-mutant cancers. Nature reviews Clinical oncology, 15 (11), 709-720.
Scharner, J., & Aznarez, I. (2021). Clinical applications of single-stranded oligonucleotides: current landscape of approved and in-development therapeutics. Molecular Therapy, 29 (2), 540-554.
Sharma, V. K., & Watts, J. K. (2015). Oligonucleotide therapeutics: chemistry, delivery and clinical progress. Future medicinal chemistry, 7 (16), 2221-2242.
Shen, W., De Hoyos, C. L., Migawa, M. T., Vickers, T. A., Sun, H., Low, A., . . . & Crooke, S. T. (2019). Chemical modification of PS-ASO therapeutics reduces cellular protein-binding and improves the therapeutic index. Nature biotechnology, 37 (6), 640-650.
Shieh, P., Nguyen, H. V. T., & Johnson, J. A. (2019). Tailored silyl ether monomers enable backbone-degradable polynorbornene-based linear, bottlebrush and star copolymers through ROMP. Nature chemistry, 11 (12), 1124-1132.
Simanshu, D. K., Nissley, D. V., & McCormick, F. (2017). RAS proteins and their regulators in human disease. Cell, 170 (1), 17-33.
Sridharan, K., & Gogtay, N. J. (2016). Therapeutic nucleic acids: current clinical status. British journal of clinical pharmacology, 82 (3), 659-672.
Swayze, E. E., Siwkowski, A. M., Wancewicz, E. V., Migawa, M. T., Wyrzykiewicz, T. K., Hung, G., . . . & Bennett, A. C. F. (2007). Antisense oligonucleotides containing locked nucleic acid improve potency but cause significant hepatotoxicity in animals. Nucleic acids research, 35 (2), 687-700.
Van Der Spoel, D., Lindahl, E., Hess, B., Groenhof, G., Mark, A. E., & Berendsen, H. J. (2005). GROMACS: fast, flexible, and free. Journal of computational chemistry, 26 (16), 1701-1718.
Veedu, R. N., & Wengel, J. (2009). Locked nucleic acid as a novel class of therapeutic agents. RNA biology, 6 (3), 321-323.
Wan, W. B., & Seth, P. P. (2016). The medicinal chemistry of therapeutic oligonucleotides. Journal of medicinal chemistry, 59 (21), 9645-9667.
Wang, D., Lin, J., Jia, F., Tan, X., Wang, Y., Sun, X., . . . & Zhang, K. (2019). Bottlebrush-architectured poly (ethylene glycol) as an efficient vector for RNA interference in vivo. Science advances, 5 (2), eaav9322.
Wang, D., Lu, X., Jia, F., Tan, X., Sun, X., Cao, X., . . . & Zhang, K. (2017). Precision tuning of DNA-and poly (ethylene glycol)-based nanoparticles via coassembly for effective antisense gene regulation. Chemistry of materials: a publication of the American Chemical Society, 29 (23), 9882.
Wang, Y., Wang, D., Jia, F., Miller, A., Tan, X., Chen, P., . . . & Zhang, K. (2020). Self-Assembled DNA-PEG Bottlebrushes Enhance Antisense Activity and Pharmacokinetics of Oligonucleotides. ACS applied materials & interfaces, 12 (41), 45830-45837.
Yakubov, L. A., Deeva, E. A., Zarytova, V. F., Ivanova, E. M., Ryte, A. S., Yurchenko, L. V., & Vlassov, V. V. (1989). Mechanism of oligonucleotide uptake by cells: involvement of specific receptors? Proceedings of the National Academy of Sciences, 86 (17), 6454-6458.
Yamamoto, T., Nakatani, M., Narukawa, K., & Obika, S. (2011). Antisense drug discovery and development. Future medicinal chemistry, 3 (3), 339-365.
Ying, H., Kimmelman, A. C., Lyssiotis, C. A., Hua, S., Chu, G. C., Fletcher-Sananikone, E., . . . & DePinho, R. A. (2012). Oncogenic KRAS maintains pancreatic tumors through regulation of anabolic glucose metabolism. Cell, 149 (3), 656-670.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of inhibiting cancer in a subject in need thereof, said method comprising administering to the subject a composition comprising: a polyethylene glycol (PEG)-conjugated antisense oligonucleotide (ASO).

2. (canceled)

3. The method of claim 1, wherein the ASO targets an oncogene.

4. The method of claim 3, wherein the oncogene is a RAS gene, and wherein the RAS gene is KRAS, HRAS, or NRAS.

5. (canceled)

6. (canceled)

7. The method of claim 1, wherein the PEG-conjugated ASO is a polymer-assisted compaction of DNA (pacDNA).

8. The method of claim 7, wherein the pacDNA is a bottlebrush polymer-ASO conjugate, and wherein the bottlebrush polymer-ASO conjugate comprises a chemically modified or unmodified ASO covalently linked to the backbone of the bottlebrush polymer.

9. (canceled)

10. (canceled)

11. (canceled)

12. (canceled)

13. The method of claim 1, wherein the ASO comprises at least about 80% sequence identity to SEQ ID NO: 1.

14. The method of claim 7, wherein the pacDNA comprises one ASO, two ASOs, or a plurality of ASOs, wherein the ASO comprises an anti-KRAS oligonucleotide.

15. The method of claim 7, wherein the pacDNA comprises at least two anti-KRAS oligonucleotides and wherein the at least two anti-KRAS oligonucleotides comprise different nucleotide sequences.

16. (canceled)

17. The method of claim 1, wherein administration of the composition is by aerosol inhalation, injection, infusion, ingestion, or a combination thereof.

18. (canceled)

19. (canceled)

20. The method of claim 1, wherein the cancer is non-small cell lung cancer, colorectal cancer, pancreatic cancer, or any combination thereof.

21. A composition comprising: a polyethylene glycol (PEG)-conjugated antisense oligonucleotide (ASO).

22. The composition of claim 21, wherein the ASO targets an oncogene.

23. The composition of claim 22, wherein the oncogene is a RAS gene.

24. The composition of claim 23, wherein the RAS gene is KRAS, HRAS, or NRAS.

25. (canceled)

26. The composition of claim 21, wherein the PEG-conjugated ASO is a polymer-assisted compaction of DNA (pacDNA).

27. The composition of claim 26, wherein the pacDNA is a bottlebrush polymer-ASO conjugate, and wherein the bottlebrush polymer-ASO conjugate comprises chemically modified ASO or unmodified ASO, wherein the ASO is covalently linked to the backbone of said bottlebrush polymer.

28. (canceled)

29. (canceled)

30. (canceled)

31. (canceled)

32. The composition of claim 21, wherein the ASO comprises at least about 80% sequence identity to SEQ ID NO: 1.

33. The composition of claim 21, wherein the ASO comprises a plurality of ASOs and wherein at least two of the anti-KRAS oligonucleotides comprise different nucleotide sequences.

34. (canceled)

35. (canceled)

36. (canceled)

37. (canceled)

38. (canceled)

39. A polymer-assisted compaction of DNA (pacDNA) comprising at least one antisense oligonucleotide (ASO) that specifically binds an oncogene mRNA.

40. The pacDNA of claim 39, wherein the oncogene is a KRAS gene.

41. (canceled)
